(19) United States
(12) Patent Application Publication
(10) Pub. No.: US 2010/0104585 A1
(43) Pub. Date: Apr. 29, 2010

(54) Methods and Reagents for Regulation of Cellular Responses in Biological Systems

(76) Inventors: Laura L. Kiessling, Madison, WI (US); Byron R. Griffith, Madison, WI (US); Jason E. Gestwicki, Mountain View, CA (US); Laura Strong, Madison, WI (US)

Correspondence Address: GREENLEE WINNER AND SULLIVAN P C, 4875 PEARL EAST CIRCLE, SUITE 200, BOULDER, CO, 80301, US

(21) Appl. No.: 12/571,983

(22) Filed: Oct. 1, 2009

Related U.S. Application Data
(63) Continuation of application No. 10/806,056, filed on Mar. 22, 2004, now abandoned.
Application No. 10/806,056 is a continuation-in-part of application No. 09/815,296, filed on Mar. 21, 2001, now abandoned.
(60) Provisional application No. 60/456,778, filed on Mar. 21, 2003.
(60) Provisional application No. 60/191,014, filed on Mar. 21, 2000.

(51) Int. Cl. A61K 39/395

(52) U.S. Cl. 424/172.1

(57) ABSTRACT

Multivalent ligands which carry or display at least one recognition element (RE), and preferably a plurality of recognition elements, for binding directly or indirectly to cells or other biological particles or more generally by binding to any biological molecule. The multivalent ligands provided can function for binding or targeting to any biological particle or molecule and particularly to targeting of cells or cell types or viruses, for cell aggregation and for macromolecular assembly of biological macromolecules. The multivalent ligands of this invention are applicable for creating scaffolds (assemblies) of chemical or biological species, including without limitation, antigens, epitopes, ligand binding groups, ligands for cell receptors and various macromolecules. In these scaffolds, the number, spacing, relative positioning and relative orientation of recognition elements can be controlled. The invention also relates to methods for aggregating biological particles and macromolecules and for modulating biological response employing the multivalent ligands provided.

### CROSS-REFERENCE TO RELATED APPLICATIONS

This application is a continuation of U.S. application Ser. No. 10/806,056, filed Mar. 22, 2004. U.S. application Ser. No. 10/806,056 claims the benefit under 35 U.S.C. 119(e) of U.S. provisional application Ser. No. 60/456,778, filed Mar. 21, 2003 and is a continuation-in-part of U.S. application Ser. No. 09/815,296, filed Mar. 21, 2001, which in turn claims the benefit under 35 U.S.C. 119(e) of U.S. provisional application Ser. No. 60/191,014, filed Mar. 21, 2000. Each of these applications is incorporated by reference herein in its entirety.

### STATEMENT REGARDING U.S. GOVERNMENT FUNDING

This invention was made at least in part with funding from the United States government through National Institute of Health grants no. GM55984 and GM49975. The United States government has certain rights in this invention.

### BACKGROUND OF THE INVENTION

A variety of biological processes are mediated by the binding of one chemical or biological species, macromolecule or particle (e.g., a cell, virus or virion) to another chemical or biological species, macromolecule or particle. In many cases there is evidence that the valency of the binding may be an important aspect of the mechanism of the mediation of the biological process. The present invention relates to compounds and methods for selectively varying the valency of such interactions employing multivalent ligands.

Multivalent ligands are chemical scaffolds, typically polymeric, to which a plurality of chemical or biological species involved in binding to other chemical or biological species (generally designated recognition elements, RE, herein) are attached in a controlled fashion, with control over the number of RE, the spacing of RE and the relative orientation of RE. Certain recognition elements (SRE, signal recognition elements) are involved directly or indirectly in biological signaling processes. Other recognition elements (BRE, binding recognition elements) are involved simply in facilitating binding that is associated with the biological process. This invention then is generally related to the control of biological processes by controlling the structure of such multivalent ligands. This invention more specifically relates to multivalent ligands in which number, spacing and relative orientation of recognition elements can be selectively optimized for a given application. Multivalent ligands herein are particularly useful as effectors in biological systems and to facilitate aggregation of biological particles (cells, viral particles, etc.) and biological molecules (saccharides, proteins, nucleic acids, lipids etc.). The multivalent ligands of this invention have applications particularly in cell signaling processes and more generally in macromolecular assembly of recognition elements that are involved in biological processes.

Cells need to continuously sense and respond to changes in their environment. For this purpose, cells use a multitude of cell surface, transmembrane and cytoplasmic receptors. These receptors typically recognize proteins, peptides, saccharides, nucleic acids, or small molecules but, in some cases, receptors may also recognize environmental changes, for example, in redox potential, temperature, and osmolarity. The binding of a ligand to these receptors results in changes in the activity of the cell such as migration, activation, metabolism, the release of chemical species, such as intercellular signals, protein production, differentiation, proliferation, cell death, and increased or decreased adhesion to other cells or to the extracellular matrix. This is a central paradigm of cell biology and these cellular responses allow the cell (or the multicellular organism) to properly respond to environmental changes.

The mechanisms by which ligands promote cellular processes are of great interest to elucidate their roles in the regulation of cellular responses. One common way in which these systems are regulated is by the spatial organization of the receptors. Ligand binding can change the relative orientations and/or conformations of the cell surface receptors, activating (or inhibiting) a response. Biological responses ranging from immune recognition, cell adhesion and migration, and proliferation, among others, rely on the reorientation or change in distribution (e.g., localization) of cell surface receptors that occurs upon ligand binding. Ligand reorientation can be the event that transmits signals (directly or indirectly) and facilitates the cellular response.

A common example of this is found in the growth factor receptors, which govern cell proliferation. Certain divalent growth factors, such as erythropoietin (EPO), bind to two cell surface growth factor receptors (EPOR) simultaneously and bring those receptors into proximity. This ligand reorganization triggers a signal transduction cascade that involves cross-phosphorylation of the receptors in the dimer. In the EPO example a ligand, which is only capable of binding to one receptor, is incapable of eliciting the response.

Signaling process may involve the interaction of multiple different receptors (e.g., co-receptors). In this case, for example, the response of a first cell surface receptor to a ligand binding event is affected by a ligand binding event at a second receptor of the cell surface. These co-receptors can enhance or attenuate the response of the first receptor. B-cell responses to antigens, for example, can be affected by co-receptors (e.g., CD38 (positive co-receptor) or CD22 or FcγRIIb1 (negative co-receptor)).

A particularly interesting feature of some ligands is valency, which herein refers generally to an interplay between the net number of recognition sites in a ligand for binding to receptors (e.g., epitopes, antigens) and the density and spacing of those sites in the ligand. Ligands often possess multiple receptor binding sites. This allows multivalent interactions between the ligand and multiple receptors which may determine the kind and intensity of biological response to that ligand. Often in these systems, monovalent ligands lack any biological activity. Researchers have explored ligands which vary in valency, at least in the sense of increasing the number of recognition sites. Typically, the ligands examined have been either small, low valency compounds, such as antibodies or dimerizing agents, or large heterogeneous compounds, such as protein conjugates, polymers, or functionalized surfaces. Work with defined low valency compounds has led to the realization of the extent of regulation by changes in receptor orientation and work with large undefined multivalent ligands has indicated that increasing the net number of recognition sites (e.g., epitopes, antigens) can often result in increased effects in many systems.

Cells require fine control over their cellular processes in order to avoid over- or under-stimulation. In the immune response, for example, immune cell function must be closely regulated to avoid unfavorable autoreactivity or clonal anergy. Cells utilize features of the interaction of receptors with ligands to regulate their responses. For example, increased synthetic ligand density has been shown to more effectively activate the response of certain cells to the ligand. Nature may utilize ligand valency to control biological responses in a defined manner. Thus, selective control of biological responses may be achieved though control of ligand valency. Previously described multivalent ligands have, however, not allowed exploration of this fine-tuned control in biological systems.

This invention provides for the generation of synthetic ligands with distinct valencies and controlled structural features which can be used to systematically alter and/or control biological responses initiated or triggered by binding to cell surface receptors. The synthetic ligands herein can also be used to generate ligand scaffolds or arrays in which the number, type, spacing and relative orientation of recognition elements is controlled (or varied in a controlled fashion). Ordered aggregates or arrays of various biological particles and or biological molecules can be generated. Such aggregate or arrays of biological molecules can, for example, be employed to initiate biological responses. In particular, the synthetic ligands of this invention allow for access to the finer control exhibited by natural ligands. Access to these features in a synthetic ligand not only expands understanding of the natural function of these systems, but also leads to selectively designed effector molecules (multivalent ligands) for use in therapeutic and non-therapeutic applications that take advantage of the ability to regulate a wide variety of biological responses.

### SUMMARY OF THE INVENTION

This invention provides multivalent ligands which carry or display at least one recognition element (RE), and preferably a plurality of recognition elements, for binding directly or indirectly to cells or other biological particles or more generally for binding to any biological molecule. The multivalent ligands provided can most generally function for binding or targeting to any biological particle or molecule and particularly for targeting of cells or cell types or viruses, for cell aggregation and generally for macromolecular assembly of biological macromolecules (including among others, saccharides, peptides, proteins, antibodies and fragments thereof, nucleic acids, small drug-like compounds and lipids).

The multivalent ligands of this invention are generally applicable for creating scaffolds (assemblies or arrays) of chemical or biological species, including without limitation, antigens, epitopes, ligand binding groups, ligands for cell receptors (cell surface receptors, transmembrane receptors and cytoplasmic receptors), and various biological molecules, including macromolecules, and specifically nucleic acids, carbohydrates, saccharides, proteins, peptides, antibodies and fragments thereof, lipids, etc. In these scaffolds, the number, spacing, relative positioning and relative orientation of RE can be controlled. Scaffolds can contain more than one covalently or non-covalently bonded RE and may contain more than one different RE. Scaffolds can comprise various chemical species, biological species and/or particles, bonded to the one or more RE.

In a more specific embodiment, multivalent ligands are provided which carry or display at least one signal recognition element (SRE), and preferably a plurality of signal recognition elements, and modulate biological responses in biological systems. Signal recognition elements provide for binding to a cell surface receptor and alone or in combination with other SRE affect a biological response in a biological system. SRE include chemical or biochemical species recognized as signals by a cell, i.e., through binding one or more cell receptors, particularly one or more cell surface receptors. These multivalent ligands can act generally as effectors of biological responses in biological systems. The multivalent ligands provided can function to activate, initiate or trigger a biological response, to inhibit a response, to enhance or attenuate a response, or to change the nature of a response. A multivalent ligand of this invention can also affect a response mediated through a cell surface receptor to which it does not itself bind.

In a more specific embodiment, multivalent ligands are provided which carry or display at least one binding recognition element (BRE), and preferably a plurality of binding recognition elements. A multivalent ligand can contain one or more different BRE, with different binding specificity or selectivity. These binding recognition elements can bind, and preferably selectively or specifically bind, to a chemical or biological molecule or a biological particle. In specific embodiments, BRE bind to biological molecules, for example, peptides or proteins, including antibodies and fragments thereof, or nucleic acids. Multivalent ligands carrying BRE can, for example, be used to aggregate, organize, or array the chemical or biological molecules or biological particles to which the BRE bind. Multivalent ligands carrying a plurality of chemical or biological molecules or biological particles bound through BRE can in turn be employed to induce a biological response. Multivalent ligands herein may combine one or more SRE with one or more BRE.

The invention provides methods for inducing a biological response in vivo, in vitro or ex vivo employing one or more of the multivalent ligands of this invention. More specifically, the invention provides methods for inducing, modulating and/or regulating biological responses in biological systems using multivalent ligands. More specifically the invention provides methods for inducing or enhancing cell aggregation or alternatively for inhibiting or preventing cell aggregation.

The invention provides methods for aggregation of biological particles, and biological molecules employing one or more of the multivalent ligands of this invention. More specifically, the invention provides methods for inducing or enhancing cell aggregation or alternatively for inhibiting or preventing cell aggregation using multivalent ligands.

Multivalent ligands herein can contain one or more functional elements (FE) which elements can include, among others, fluorescent or other optically detectible labels or radiolabels and isotopically-labeled tags.

Preferred multivalent ligands of this invention have defined or controlled valency, in which structural features of the ligand are selected or controlled, including the number, density, spacing and orientation of recognition elements (RE, SRE or BRE and optionally FE) for binding to receptors, to simply bind to a cell or to obtain a desired type of biological response or level of response.

Scaffolded multivalent ligands of this invention which comprise a plurality of RE, SRE, BRE or mixtures thereof, optionally in combination with FE, can be employed in a variety of diagnostic and clinical applications, in particular in blood typing, in pathogen detection, pathogen clearance, detection of tumor cells, sensitive detection of tumor antigens, or detection of foreign macromolecules, for example detection of foreign proteins, or foreign carbohydrates. The multivalent ligands herein can be employed in the detection of various biological molecules and particles (cells and viruses) and in a variety of assay methods (histology, Western blots, PCR assays, ELISA assays, agglutination assays, among others). In general, increases in valency in such ligands will be associated with an increase in assay or diagnostic sensitivity.

Multivalent ligands comprise one or more structural or functional groups which act as recognition elements (RE) for binding to cell surface receptors. Multivalent ligands can comprise one or more structural or functional groups which are BRE and/or SRE and optionally one or more functional elements (FE). SREs are a subset of REs that, alone or in combination with other SREs (BREs or FEs) in a multivalent ligand, can induce intracellular and/or intercellular biological responses. Multivalent ligands of this invention carrying one or more SRE (optionally in combination with one or more BRE, which may be different BRE, one or more different SRE or one or more FE) can initiate a biological response in a cell. Alternatively, these multivalent ligands can modulate the response of a cell in the presence of one or more natural chemical or biochemical signals, for example, by enhancing, decreasing or inhibiting the response. In specific embodiments, multivalent ligands of this invention are designed to change the level or type of response that is induced in a cell by a selected chemical or biochemical signal.

Multivalent ligands of this invention most generally comprise a molecular scaffold to which a plurality of REs (BREs, SREs or both, optionally in combination with FEs) are bonded either by covalent or non-covalent interactions. The number, density and spacing of the BRE, SRE and FE on the scaffold can be controlled, typically by selective synthesis of desired ligands. The molecular scaffold can be linear, branched or cyclic providing different geometries of presentation of BRE and/or SREs to cells.

In preferred embodiments, molecular scaffolds are polymers comprising a plurality of monomers. Molecular scaffold of the multivalent ligands of this invention include polymers in which all of the monomers are the same or copolymers containing a mixture of different monomers. Molecular scaffolds can also include block copolymers in which different regions (or portions) of the scaffold are composed of different monomers. Molecular scaffolds prepared by ROMP methods, and by atom-transfer radical polymerization (ATRP), as illustrated in several formulas herein, are preferred.

Molecular scaffolds can be hydrophobic or can be made to be more hydrophilic by substitution (particularly of the polymer backbone) with polar substituents, such as —OH. The scaffold can be substituted, in general, with any groups that do not interfere with BRE or SRE activity, e.g. binding to a receptor. Substitution of the scaffold can be controlled to adjust the physical properties, e.g., solubility, of the multivalent ligand. BREs, SREs and FEs may be directly attached to a scaffold or attached to the scaffold via linker groups. The linker group provides functional groups for bonding to the scaffold and for bonding to BREs, SREs and/or FEs and can also affect solubility of the multivalent ligand. The linker can also provide a defined spacer to minimize undesired interactions among BREs, SREs or FEs or between the attached elements and the scaffold or to provide structural flexibility with respect to orientation of attached elements.

In specific embodiments, the molecular scaffold comprises a plurality of repeated units (monomers) to each of which an BRE or SRE is bonded. In general, the molecular scaffold functions to hold the signals in proximity to each other and does not interact directly in the modulation of the biological response. However, physical (e.g., solubility) or chemical (e.g., stability) properties of the multivalent ligands can be varied by selection of the structure of the scaffold or by introducing substituents (e.g., polar, non-polar) along the scaffold.

In one embodiment, the multivalent ligands have only one type of BRE or SRE in the ligand. These multivalent ligands include dimers, trimers, tetramers and polymers (including relatively short oligomers having 5 or more monomers) or longer polymers having 25, 50, 100, 200, 300 or more monomers. Preferred multivalent ligands carrying one type of BRE or SRE carry about 10 or more of such BREs or SREs. In this embodiment, the repeating units (or monomers) of the multivalent ligand are preferably the same.

In another embodiment, the invention provides multivalent ligands that carry more than one type of BRE, more than one type of SRE or a combination of BRE and SRE. These multivalent ligands also include dimers (carrying one of each BRE or SRE or a BRE and an SRE), trimers, tetramers and block polymers (including relatively short oligomers having 5 monomers or more) or longer polymers having 25, 50, 100, 200, 300 or more monomers. These multivalent ligands may also have spacer regions (with monomers that do not carry any BRE or SRE group) along the scaffold to separate regions carrying a first BRE or SRE from regions carrying a second BRE or SRE. Monomers in spacer regions may carry a functional element (FE), may be unsubstituted or may carry a non-reactive, non-functional group. A given multivalent ligand can generally contain any number of different BREs, SREs, or both. However, those carrying 2 or 3 different BRE or SRE are of most interest.

In other embodiments, the invention provides multivalent ligands that carry one or more BRE or SRE, which may be the same or different, but also carry functional elements (FE) other than BRE or SRE. These functional elements (FE) can, for example, exhibit a variety of chemical or biochemical functions (different from those of BREs or SREs). They can, for example, provide one or more fluorescent (or other optical label) or radiolabels, provide one or more groups with latent reactive groups, or provide one or more enzymatic functions. Substitution of monomers with FEs can also provide for spacing of BREs or SREs. The invention provides methods for labeling or targeting of cells with functional elements (FE). In these methods, RE of the multivalent ligand, particularly BRE, function for bonding to the molecule or particle to be labeled with one or more FE.

Recognition elements (RE) are any chemical or biological species (e.g., molecules or portions thereof) that alone or in combination with one or more other REs, recognize and bind to a chemical or biological molecule or species or particles. In specific embodiments, RE bind to cell surface receptors RE can, for example, include all or a portion of a ligand active for binding to a cell surface receptor. Signal recognition elements (SRE) are any chemical or biochemical species that, alone or in combination with one or more other SREs, induce a biological response in or from a cell and include biological molecules (proteins, glycoproteins, peptides, amino acids, nucleic acids, saccharides, cytokines, growth factors, hormones, and various derivatives thereof) and which may be portions of larger biological species (protein fragments, antibody or antibody fragment, epitopes, antigenic determinant, etc.) and various chemical species (haptens, naturally-occurring small molecules, synthetic small molecules, small drug-like molecules, particularly those with known therapeutic effects) and species that act as functional mimics of biological molecules (e.g., peptoids, phosphorothioates). In specific embodiments, SRE are RE which bind to a cell surface receptor and directly or indirectly induce a biological response. In contrast, BRE are RE which participate in bonding (alone or in combination with other BRE or SRE), but which do not themselves alone or in concert with each other affect a biological response in the cell. BRE, in general, function for aggregation of other molecules or particles, particularly biological molecules and particles. In specific embodiments, multivalent ligands carrying BRE groups function for aggregation of biological molecules, particularly peptides and proteins, including antibodies and fragments thereof.

Multivalent ligands of this invention can function to reorganize and/or cluster cell receptors. In this regard the BRE or SRE on the multivalent ligand will be a ligand of the cell receptor. In certain cases, clustering or reorganization of receptors modulates the cell's response to a given SRE. Clustering or reorganization of receptors by a multivalent ligand of this invention can also modulate the response of a cell to another signal or another ligand. Through clustering or other structural reorientation or reorganization of cell surface receptors, a multivalent ligand of this invention can enhance or inhibit the cell's response to another signal or ligand. For example, multivalent ligands of this invention that function as chemoattractants can enhance the response of a cell to another chemoattractant.

A given cell receptor may mediate more than one biological response. The multivalent ligands of this invention that carry ligands which bind to a given cell receptor, but which do not induce a biological response mediated by that receptor, may be employed to inhibit the biological response.

Multivalent ligands that carry more than one type of SRE (or SRE in combination with BRE) can be used to simultaneously or sequentially induce more than one biological response in or from a cell. Alternatively, the cellular response to one SRE can be modified by the cellular response to another SRE. Multivalent ligands carrying two or more different SREs can function, for example, to reorganize different receptors on the cell surface, which can result in modulation of cellular response to one or more SREs. Similarly, the binding of one BRE may modify the cellular response induced by a SRE. Further, in multivalent ligands carrying FE, in addition to one or more SRE, the response to an SRE can be modified by the presence of FE.

Multivalent ligands of this invention can be employed in methods to modulate signal transduction processes (i.e., the transmission of information between the outside and the inside of a cell and between cells, in biological systems) in prokaryotic or eukaryotic cells. The methods can be practiced in vivo, in vitro or ex vivo (where cells are removed from a natural environment, including a multicellular organism, and are intended once treated to be returned to that environment). For example, chemotaxis or cell migration responses to SREs can be modulated. Such methods are applicable to prokaryotes (e.g., Gram negative, Gram positive bacteria as well as archeabacteria), eukaryotic microorganisms (including, without limitation, fungi, eukaryotic parasites and pathogens of various organisms (including mammals, particularly humans), and eukaryotic cells of larger organisms including those of mammals, and specifically including those of humans (e.g., hematopoietic cells, stem cells, blood cells, leukocytes, lymphocytes, endothelial cells, epithelial cells, mature cells, differentiated cells, liver cells, muscle cells, cancer cells, neuronal cells, dendritic cells, natural killer cells, cardiac myocytes, adipocytes, etc.) Multivalent ligands that modulate responses in bacterial cells or in eukaryotic cells, including eukaryotic pathogens or parasites, can be used to inhibit proliferation, colonization, migration, or biofilm formation by the bacterium, or eukaryotic pathogen or parasite and, as a consequence, can inhibit infection or colonization by such microorganisms.

Multivalent ligands can also be used to promote or inhibit cell differentiation, cell proliferation and/or cell death (e.g., apoptosis), particularly in mammalian cells, including human cells. Multivalent ligands that modulate responses in eukaryotic cells of larger organisms can be used to inhibit undesired cell proliferation, undesired migration and undesired formation of cell to cell junctions or to promote or enhance desired cell proliferation, desired migration and desired formation of cell junctions dependent upon the selection of SRE and other FE in the multivalent ligand.

The invention provides multivalent ligands, pharmaceutical and/or therapeutic compositions comprising one or more multivalent ligands, methods for making multivalent ligands and method for using multivalent ligands, particularly methods for aggregation of biological molecules or biological particles employing multivalent ligands.

Pharmaceutical and therapeutic compositions which comprise a pharmaceutically acceptable carrier and an amount of a multivalent ligand effective for modulating cell proliferation, colonization, migration, cell to cell junction formation and/or biofilm formation by eukaryotic or prokaryotic cells are encompassed by this invention. Specific pharmaceutical or therapeutic compositions include those which comprise an amount of a multivalent ligand effective for inhibiting or disrupting undesired cell proliferation, colonization, migration, cell to cell junction formation and/or biofilm formation by eukaryotic or prokaryotic cells. Pharmaceutical compositions that retard or inhibit infections by bacteria or eukaryotic parasites or pathogens are of particular interest. Two or more multivalent ligands of this invention can be combined in such pharmaceutical compositions to provide for combined effect and benefit.

Cell migration, adhesion and the formation of cell to cell junctions are involved in cancer growth and metastasis. Multivalent ligands that modulate such processes can be employed in methods and pharmaceutical compositions for inhibition of cancer growth and metastasis. Again such pharmaceutical compositions include those which comprise an amount of a multivalent ligand that is effective for inhibiting cancer cell growth, adhesion or migration. Two or more multivalent ligands of this invention can be combined in such pharmaceutical compositions to provide for combined effect and benefit.

Multivalent ligands of this invention can modulate immune responses in animals (including mammals and particularly in humans) by valency-dependent interaction with cells that function in the immune system (e.g., leukocytes and lymphocytes). In particular, multivalent ligands of this invention can modulate the response of leukocytes, including neutrophils, to chemoattractants (including derivatized peptides, such as N-formyl peptides, and N-acyl peptides) and can modulate the activation and deactivation of B-cells and/or T-cells. B-cell and/or T-cell activation can be performed in vivo, in vitro and/or ex vivo.

The invention also provides libraries of multivalent ligands in which the members of the libraries are varied, for example, in the type, number and/or relative positioning of RE (BRE and/or SRE), combinations of BRE and SRE, the presence and/or positioning of spacers, in the number of repeating units or monomers (e.g., n or n+m in formulas below) and in the presence, type or number of FE. Libraries of multivalent ligands which span a range of defined sizes, numbers of repeating units or monomers, numbers of BRE or SRE, combinations of BRE or SRE, combinations of BRE, SRE and FE and spacing of attached elements, (BRE, SRE and any FE) are of particular interest. Libraries prepared using ROMP-methods are of particular interest and application. Libraries prepared using ATRP methods are also of particular use and application. Libraries can optionally be formed by attachment of multivalent ligand library members to solid supports, e.g., to particles or to substrate surfaces. Multivalent ligand library membranes may be attached to such particles or substrates in an organized fashion to facilitate library screening. Multivalent ligands may be grouped on particles or surfaces according to a defined structural or chemical relationship among the grouped multivalent ligands (e.g., ligand length, number of REs, types of SRE, etc.).

Using various selection and screening methods that are understood in the art, these libraries can be selected or screened for multivalent ligands in the library which exhibit desired modulation in a given biological system. Furthermore, the results obtained from such screens, i.e., the number of BRE required for cell aggregation, the number of SRE's required for induction or inhibition, and other structure/function relationships, can be used in the design and synthesis of additional multivalent ligands. In a specific embodiment, multivalent ligand libraries can be screened for enhanced binding to a selected cell receptor.

### BRIEF DESCRIPTION OF THE DRAWINGS

FIG. 1 schematically illustrates several ways in which multivalent ligands of this invention can function in macromolecular assembly and as effectors of biological responses.

FIG. 2A: Results of video microscopy motion analysis experiments. Bacteria (Escherichia coli) were treated with buffer alone, galactose, or compound 1 or 3 (Scheme 1) at the indicated saccharide concentrations. The results represent the average from at least five independent experiments performed in triplicate. Error bars represent the deviation between per-second averages during the ten second interval.

FIGS. 2B-E: Selected sample paths for bacteria (Gram Negative, E. coli) treated with buffer alone (B); 1 mM galactose (C); or 1 mM compound 1 (D); or 1 mM compound 3 (E). Sample paths are derived from motion of representative bacteria from a treated bacterial population.

FIGS. 3A and 3B: Results of E. coli capillary accumulation assays. The number of bacteria accumulated is plotted versus the concentration of the attractant (galactose or compounds 1-4, Scheme 1) calculated on a saccharide residue basis. (A): Results are shown for capillaries filled with buffer alone, compound 1, and compound 2 or (B): buffer alone, compound 3 and compound 4 at the indicated concentrations. The vertical line at 1 mM indicates the concentration of maximum chemotaxis for the monomeric compound 1. The concentrations used in this assay are not directly comparable to those used in the motion analysis experiments (see FIG. 2A), because the gradient formed in the capillary assay is not defined. Results are the average of 3 to 6 experiments performed in duplicate and error bars represent a single standard deviation. Partial permeabilization was required to obtain chemotaxis towards 4, and was utilized for all experiments as described in The Examples.

FIG. 4: Results of B. subtilis capillary accumulation assays using ROMP-derived glucose ligands (compound 5-7, Scheme 1). Buffer alone, glucose, or glucose-bearing compounds 5-7 were used as attractants in the capillary accumulation assay. Results are shown for glucose, compound 5, compound 6, and compound 7. Results are the average of at least four trials performed in duplicate and error bars represent single standard deviations.

FIGS. 5A and 5B: Results of video microscopy motion analysis experiments. (A): Bacteria (E. coli) were treated with increasing concentrations of serine (μM) after initial treatment (followed by a 2 min adaptation period) with buffer alone (▪) or 10 μM attractant: galactose (), compound 1 (10mer, ▴) or compound 3 (25 mer, ♦); (B) Bar graph of data for angular mean velocity taken from FIG. 5A at serine concentration 1 μM. Initial treatment with compound 3 results in a significant enhancement of bacterial response to serine. Angular mean velocities varied approximately 14% between experiments performed on different days.

FIG. 6: Multivalent ligands bind specifically to chemoreceptors and induce receptor reorganization. The illustration schematic represents fluorescently labeled 8 (10, 590 nm emission) bound to Trg (11) via glucose/galactose binding protein (GGBP) (12). Trg is labeled with a fluorophore-tagged anti-Trg antibody (13, 530 nm emission).

FIGS. 7A-D: Model of receptor reorganization by synthetic ligands. (A) Chemoreceptors are observed to form dimers (or multimers) (20) in the plasma membrane of E. coli and each dimer appears to interact with a single periplasmic binding protein (21) [59, 60]. Monovalent galactose ligands, such as galactose and compound 1 (22), interact with Trg through GGBP binding, inducing signal transduction from chemoreceptor dimers; (B) Multivalent galactose compounds, such as compound 2, that cannot span the distance needed to reorganize the receptors (23) generate signals from individual dimers, as in (A); (C) Multivalent ligands of sufficient lengths (24), such as compounds 3 and 4, are able to reorganize the chemoreceptors into discrete clusters (25) at the plasma membrane; (D) Extending the valency of a multivalent ligand (26) likely increases the extent of reorganization and, therefore, the bacterial response.

FIG. 8: illustrates various designs for molecular scaffolds that can be employed in the multivalent ligands of this invention. These types of scaffolds can be constructed, for example, employing alicyclic or aromatic (including heteroaromatic) ring systems and combinations thereof. Scaffolds provide the geometry of presentation of two or more RE, which can be BREs, SREs or both. Linkers may have varying structures and, for example, be rigid, flexible or branched. In each of the illustrated structures any of a rigid, flexible or branched linker can be employed. Each branched linker may be attached to more than one RE, SRE (or FE). In each structure, one or more FE (so long as at least one BRE or SRE remains) can replace one or more BRE or SRE.

FIGS. 9A-C: Illustrate models of the ability of multivalent ligands to activate or inhibit cell aggregation in a valency- and concentration-dependent fashion; (A) Monovalent ligands (31) (such as 9) are necessarily inhibitory if they bind to concanavalin A (ConA) (30); (B) Multivalent ligands 32 (such as 12) at sufficiently low concentrations and optimal stoichiometry with ConA may allow cell aggregation (34), despite their occupation of ConA binding sites; (C) At increased concentrations of multivalent ligands 32 (approximately 5 μM in the case of 10-12) ConA sites become saturated (35), disassembling clusters and inhibiting cell aggregation.

FIG. 10: Bar graph illustrating that ConA clusters assembled on ROMP-derived scaffolds are able to form aggregates of Jurkat cells. Percent of Jurkat cells present in aggregates is plotted against the treatment. ConA at 100 μg/mL or 5 μg/mL is able to form aggregates. Aggregate formation could be inhibited by addition of 50 mM methyl α-D-mannopyranoside (α man). Compounds 9-12 were added to a final mannose concentration of 0.5 μM or 5 μM along with a final ConA concentration of 5 μg/mL. Results are the average of at least three independent experiments and error bars represent single standard deviations.

FIG. 11: Controlling ConA-mediated erythrocyte agglutination. A graph of macroscopic aggregation index (% MAI) as a function of time after contact with cells (sec) for treatments with ConA alone or ConA in combination with ligand compound 13 (Scheme 1, mannose containing ligand with n=100). The concentration of ConA used was 5 μg/mL (53 nM, based on ConA tetramer) and ligand (530 nM, based on saccharide). Thus, the ratio of mannose (in the ligand) to ConA tetramer in the experiment was 10:1. Addition of the multivalent ligand significantly enhanced erythrocyte agglutination.

FIG. 12: Enhancement of Cell Toxicity of ConA by a Multivalent Ligand. A bar graph indicating % cell viability of PC12 cells as a function of various treatments. “HBS” is the medium control; “ConA” is treatment with 0.1 μM ConA (based on ConA tetramer) in HBS medium; “Compound 11” is treatment with 4 μM compound 11 (concentration based on saccharide) in HBS; “ConA+Compound 11” is treatment with 0.1 μM ConA and 4 μM compound 11 in HBS. Addition of the multivalent ligand, which binds to ConA, significantly enhances ConA toxicity.

FIG. 13 is a schematic illustration of oligomerization of His-tagged proteins on a nickel-chelating polymer scaffold.

FIG. 14 is a graph of proliferation of BaF3 cells in response to FGF-8b+polymer in the absence of heparin/heparan sulfate. Cells were treated as shown and incubated at 37° C. for 48-72 hours. Proliferation was measured using a modified MTT assay (CellTiter 96 Aqueous One, Promega).

FIGS. 15A and 15B are illustrations of SDS-PAGE-gels (12%, under reducing conditions) comparing cross-linking of FGF-8b mediated by nickel-chelating polymer (FIG. 15A) and heparin (FIG. 15B). FGF-8b was incubated with polymer 6 for 1 hour at 0° C. The ratio of FGF-8b to polymer was as follows: a. 1:0.001, b. 1:0.003, c. 1:0.01, d. 1:0.03, e. 1:0.1, f. 1:0.3, f. 1:1. EGS (ethylene glycol bis[succinimidylsuccinate]) was added, and cross-linking was allowed to proceed for 5 minutes at 0° C. The reactions were quenched with ethanolamine (excess) for 1 hour at 0° C. Samples were concentrated under vacuum, analyzed on a 12% SDS-PAGE gel under reducing conditions, and detected with Coomassie staining.

FIG. 16 is a graph of results of assays of the toxicity of the nickel-chelating polymer. Addition of 100 nM polymer 6 to IL3-containing media caused no significant decrease in cell viability.

FIG. 17 is schematic illustration of the construction of bifunctional conjugates for immune adherence based on a linear polymeric scaffold.

FIG. 18 is a schematic illustration of immune adherence.

FIG. 19 is a schematic illustration of pathogen clearance.

FIG. 20 is a schematic illustration of non-specifically cross-linked bispecific antibodies for pathogen clearance (from Taylor et al.)

FIG. 21 is a schematic illustration of Fab′ fragments of two different specificities conjugated to a polymeric scaffold chemoselectively.

FIGS. 22A and 22B are graphs providing a comparison of bifunctional conjugates of this invention with antibody-based conjugates (see text). FIG. 22A compares molecular weight versus valency (# of binding epitopes). FIG. 22B compares binding site density versus valency.

FIG. 23 illustrates the preparation of Fab′ fragments from IgG using pepsin and cysteamine.

FIGS. 24A and 24 B are gels illustrating characteristics of Fab′/polymer conjugates. FIG. 24A demonstrates that the “150” mer can accommodate at least about 9-10 Fab′ fragments. FIG. 24B demonstrates that the conjugation is completely chemoselective.

### DETAILED DESCRIPTION OF THE INVENTION

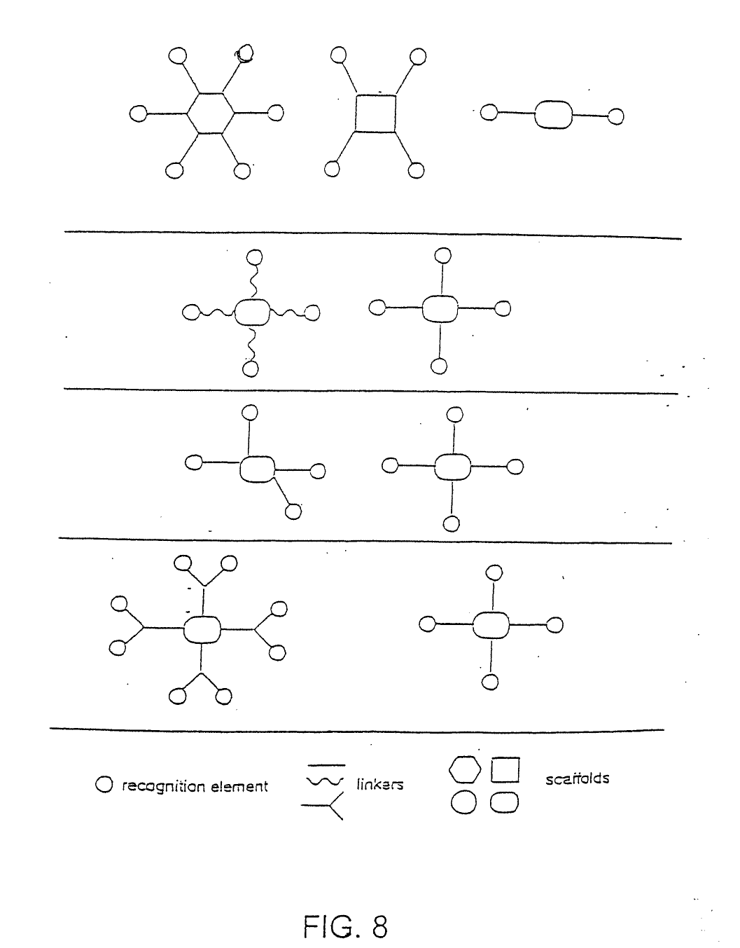

The multivalent ligands of this invention are molecular scaffolds to which a plurality of functional or structural groups, particularly BRE and/or SREs, are bonded, to present a display of the functional or structural groups in a productive manner. The scaffold can, in general, be formed from any chemical or biological species that provides the desired orientation of display. In addition to linear arrays, the scaffolds can be chosen to provide arrays of functional groups with selected non-linear presentation. See, for example, the various non-linear scaffold structures illustrated in FIG. 8.

The functional or structural groups may be bonded to the scaffold in a symmetric or unsymmetric array. The scaffold may comprise a relatively small organic molecule, such as an aromatic ring system (including benzene, naphthalene and fused and non-fused aromatics). Various fused aromatic systems can provide a wide range of different display orientations with functional groups bonded at selected positions on the ring system. Alternatively, saturated ring systems (e.g., cyclohexanes), heterocycles (e.g., carbohydrates), or alicyclic compounds (e.g., tris(hydroxymethyl)aminomethane) can also be used. Molecular scaffolds more typically comprise a plurality of repeating units or monomers, e.g., are polymers or oligomers. The molecular scaffold then carries a plurality of functional or structural groups bonded to repeating units or monomers. The functional groups are bonded covalently or noncovalently to the scaffold and can comprise a plurality of recognition elements (RE). The functional groups are bonded covalently or noncovalently to the scaffold and can comprise a plurality of binding recognition elements (BRE), or signal recognition elements (SRE), and can optionally comprise other functional elements (FE).

The RE, SRE and any FE can be bonded on to the molecular scaffold randomly or to a pre-selected pattern in which the arraignment of the RE, SRE and FE along the length of the scaffold matches a selected pattern, e.g., alternating different SRE or RE, selected spacing of different SRE or RE and the like.

The molecular scaffold can be rigid or flexible, hydrophilic or hydrophobic, symmetrical or unsymmetrical, have large surface area or small surface area, and interact or not with cell surface receptors. The molecular scaffold can be any of a variety of oligomers or polymers, including without limitation, polyacrylamides, polyesters, polyethers, polymethacrylates, polyols, and polyamino acids and corresponding oligomers. Molecular scaffolds can in general be linear polymers, branched polymers or cross-linked polymers. Preferred molecular scaffolds are biocompatible.

Molecular scaffolds prepared by ROMP methods, as illustrated in several formulas herein, are preferred. Molecular scaffolds prepared by atom-transfer radical polymerization (ATRP) which provides polymers of uniform molecular weights are also preferred. Methods for ATRP synthesis are described, for example, in WO 01/18080 and Godwin et al., 2001. Molecular scaffolds can be hydrophobic or can be made to be more hydrophilic by substitution with polar substituents, such as —OH. The scaffold can be substituted, in general, with any groups that do not interfere with signal activity and which provide desirable chemical and physical properties.

The term “recognition element” or RE is used herein to refer to chemical or biochemical species, groups or structures that functions for binding to a chemical or biological molecule or particle, including cell receptors, and more particularly which function or binding to cell surface receptors. RE are bonded to molecular scaffolds in the multivalent ligands of this invention. An RE can be a ligand for a cell receptor or a portion of such a ligand that is functional for receptor binding and that has been modified to allow its bonding to a molecular scaffold. An RE can be chemically identical to a cell receptor ligand or it may be modified from the ligand as a result of or to facilitate bonding to the scaffold.

The term “signal recognition element” or SRE is used herein to refer to chemical or biochemical species, groups or structures that function as chemical or biochemical signals (see below) and that are bonded into multivalent ligands of this invention. The SRE is typically a signal (group or molecule) that has been modified to allow its bonding into the multivalent ligand. An SRE can be chemically identical to a signal or it may be modified from the signal as a result of or to facilitate bonding to the scaffold. The SRE is preferably bonded into the multivalent ligand such that the signal function of the group is minimally affected. SREs may be recognized by cells, typically by binding to a cell receptor. SREs, in induce a response in or from cell. The response may be an intracellular response, such as cell migration, and/or an intercellular response, such as the release of chemical species by the cell (e.g., a hormone or ionic species, such as Ca²⁺) that function as chemical signals for other cells. Signal recognition is mediated by the presence of cell receptors on the cell surface to which the signal (or signal group) binds. Binding of signal (or SRE) alone may induce the biological response. Induction of the response may in some cases require presentation of multiple signals or (SRE). The biological response may in some cases be modulated by reorganization of receptors, clustering of receptors, or clustering of more than one different receptor on the cell surface.

The term “binding recognition element” or BRE is used herein to refer to is used herein to refer to chemical or biochemical species, groups or structures that function for binding to molecules (e.g., proteins, antibodies or antibody fragments, peptides, nucleic acids, small drug-like compounds, lipids, etc.) or biological particles (e.g., cells, viruses, etc.) and that are bonded into multivalent ligands of this invention. BRE do not exhibit the signaling function of SRE, however the molecules and/or particles to which BRE bind may exhibit signaling function. BRE may be recognized by and bind to cell surface receptors. Binding to a given biological particle may involve binding of one or more than one BRE.

The term “chemical or biochemical signal” is used herein to refer to a particular chemical or biochemical species selected from various types (molecules, oligomers, moieties, groups etc.) that are recognized by a cell most typically by interaction with a cell surface receptor, and induce a biological response in the cell. A signal itself can induce the response on interaction with the cell or may only induce the response when multiple signals interact (e.g., when presented multivalently) with the cell. Signals can include the natural signals, which are those species found in vivo in a biological system to induce a response in or by a cell. Natural signals include, for example, natural products, hormones, antigens, grow factors, cytokines, proteins, peptides, derivatized peptides (e.g., sulfated, phosphorylated, acylated, or N-formylated peptides), antibodies and fragments thereof, saccharides, derivatized saccharides (e.g., sulfated, acetylated, sialylated), nucleic acids, various cell nutrients, epitopes and various small organic compounds (all of which may not represent mutually exclusive groups). Signals can also include chemical species that are found to mimic the function of natural chemical signals. These signal mimics are typically synthetic and can include, for example, synthetic drugs-like compounds and various derivatives of naturally-occurring signals (e.g., peptoids and nucleic acid analogs or derivatives). Different cells can, of course, recognize different signals. Different cells may respond to the same or similar signals, with the same or with different biological responses. A single cell may respond to a plurality of different signals to give the same or different biological response. Signals include, for example, chemoattractants and epitopes (antigenic determinants) which are not mutually exclusive groups. SREs bound to multivalent ligands can comprise a chemical or biochemical signal adapted for bonding to a molecular scaffold. SREs can include, among others, chemical and biochemical species that are chemoattractants, epitopes, cytokines, hormones and related substances.

A chemoattractant is a chemical or biological signal toward which a cell migrates. The cell senses increasing concentrations of the chemoattractant and moves toward higher concentrations. Cell sensing mechanisms for chemoattractants are often very sensitive. Alternatively, cells may, in response to other signals, move to lower concentrations of signal. Bacterial cells migrate toward certain nutrients, such as glucose or galactose or amino acids, such as serine. Leukocytes (white blood cells) migrate toward, N-formyl peptides and other derivatized peptides, the activated component of C5 (C5a), platelet-activating factor (PAF), leukotriene B4 (LTB4), or chemotactic cytokines (i.e., chemokines, including α- and β-chemokines) (65). N-formylated peptides are products of bacterial protein synthesis and signal bacterial infection. The receptors for N-formylated peptides may also bind to other derivatized peptides such as N-acyl-peptides. Thus, any ligand (which may include species that act as agonist or antagonists of receptor function) of an N-formylated peptide receptor may be employed for applications related to that receptor. Neutrophils, one type of leukocyte, are guided to the site of bacterial infection by sensing low levels of N-formylated peptides. Once at the site of infection phagocytosis can occur. A chemoattractant may induce biological responses in addition to migration or chemotaxis. For example, in various types of leukocytes, chemoattractants can induce the release of toxic species or the release of inflammatory cytokines, transcription factors and other chemical species which, in turn, function as chemical signals for other cells.

The term antigen is used broadly herein as it is understood in the art and includes any non-self molecule capable of eliciting antibody formation. The term epitope is used broadly herein as it is understood in the art and includes any chemical species that functions as an antigenic determinant. The term antigen RE of this invention can include antigens and epitopes. Antigens and epitopes can be BRE or SRE. Epitopes are those parts of an antigen that combine with an antigen-binding site on an antibody molecule or on a lymphocyte (e.g., B cells and T cells) receptor. Binding of the epitope can, for example, stimulate antibody production or T cell responses. Epitopes may exhibit different levels of immunogenicity. Those that are more immunogenic than others and which dominant the overall antigenic response are designated immunodominant epitopes. Most non-self proteins and many carbohydrates are antigens, so epitopes include, without limitation, protein fragments (e.g., peptides) and carbohydrates (e.g., saccharides, glycolipids, glycopeptides and oligosaccharides). As used herein the term “self” as applied to antigens, epitopes or cells is an entity that is recognized by an immune cell, a combination of immune cells or an immune system as self. The term “self” may also be applied other biological particles that are recognized as self by an immune cell, or cells or an immune system. Some antigens, epitopes, cells and particles that are recognized as self are actually foreign to the immune cell, cells or immune system, but are not so recognized. As used herein the term “foreign” as applied to antigen, epitope or cell is an entity that is recognized by an immune cell, a combination of immune cells or an immune system as foreign. Foreign is also any thing that is not recognized as self, i.e., non-self antigens, etc. The term “foreign” may also be applied to other biological particles that are recognized as foreign by an immune cell, or cells or an immune system. Some antigens, epitopes, cells and particles that are recognized as foreign are actually self to the immune cell, cells or immune system, but are not so recognized.

The term hapten takes its generally accepted meaning in the art as a small molecule, having at least one of the determinant groups of an antigen, that can combine with an antibody but is not immunogenic unless it acts in conjunction with a carrier molecule. Haptens include, among others, hemocyanins and nitro-substituted aromatic compounds, such as dinitrophenyl groups, trinitrobenzene sulphonyl groups, and dinitrofluorophenyl groups.

The term antibody as used herein is intended to encompass any protein or protein fragments that function as an antibody and is specifically intended to include antibody fragments including, among others, Fab′ fragments.

In specific embodiments, the multivalent ligands of this invention (including both ROMP and ATRP polymers) can contain BRE and SRE that are antibodies and/or fragments thereof.

The following discusses the generation of antibody fragments. The basic unit from which all antibody molecules are formed was elucidated by Porter (1959) Biochem J. 73, 119-126, using specific proteolytic enzymes. A particularly useful immunoglobulin, IgG, comprises two heavy and two light chains with the former being coupled at their hinge region by disulfide linkages. Cleavage with papain above these linkages releases two antibody binding fragments (Fab) and a crystalline fragment (Fc). Cleavage with pepsin below the hinge results in a somewhat smaller Fc fragment and a single F(ab′)₂ fragment with two binding sites. Each Fab fragment contains both a light chain and part of a heavy chain, and includes the sequences responsible for specific binding to an antigen. The Fc portion consists of the remainder of the two heavy chains and has effector functions, e.g. relating to binding and function of complement, macrophages and polymorphonuclear white blood cells. The two heavy chains (but not the light chains) are different for each class of antibody, e.g. IgG, IgM, IgD, IgA and IgE.

Fabs are produced from polyclonal or monoclonal antibody preparations. A monoclonal antibody preparation can be derived from techniques involving hybridomas and recombinant techniques. Various expression, preparation, and purification methodologies can be used as known in the art. For example, microbial expression of antibodies can be employed (e.g., see U.S. Pat. No. 5,648,237). Human, humanized, and other chimeric antibodies can be produced.

Starting with polyclonal serum or hybridoma supernatant, purified immunoglobulin is digested with papain followed by purification of the Fab away from the Fc fragments generated in the digest. Commercial kits are available such as for preparation of Fab fragments from IgG (Pierce Product No. 44885; Pierce Biotechnology, Rockford, Ill.).

Alternatively, Fab′ molecules are generated by using pepsin digestion of F(ab′)2 fragments followed by reduction of disulfide linkage between the heavy chains, for example with cysteamine. F(ab′)₂ fragments are prepared by pepsin digestion (Sigma Chemical Co., St. Louis, Mo.) at a 3:100 (wt/wt) ratio of pepsin/IgG and incubated at 37° C. in 0.2 M acetate buffer pH 4.0 for 4 to 24 hours, followed by gel filtration on a Superdex 200 column (Pharmacia, Uppsala, Sweden). Fab′ fragments are then obtained by reduction of the F(ab′)2 with 10 mM cysteamine (Fluka, Buchs, Switzerland) for 1 h at 37° C. in Hepes/NaCl buffer pH 7.0, followed by separation on Sephadex G25-PD10 columns (Pharmacia).

Using recombinant techniques, Fab or Fab′ molecules are generated by introduction of a stop codon in the heavy chain gene at a desired location. For Fab molecules, the location can be within the hinge region at approximately the codon for the amino acid at which papain digestion occurs. For Fab′ molecules, the location can approximate the pepsin cleavage point. The Fab′ or Fab is then produced directly by simultaneous expression of both the light chain and engineered heavy chain genes to produced their respective proteins which assemble and are secreted from the cell.

In addition to Fab′ and Fab molecules, other recognition molecules are suitable for use with the invention. Such recognition molecules can include antibody-like molecules, antibody-derived molecules, and other molecules. For example, single chain antibody variable region fragments (scFv) are employed. Furthermore, hybrid molecules such as bispecific Fab-scFv (“bibody”) and trispecific Fab-(scFv) (2) (“tribody”) heterodimers or multimers can be employed (Schoonjans R et al., (2000) Fab chains as an efficient heterodimerization scaffold for the production of recombinant bispecific and trispecific antibody derivatives, J Immunol. Dec. 15; 165(12):7050-7). In connection with the invention, scFv can be prepared with or without disulfide linkages. See: Worn A, Pluckthun A., (1988) An intrinsically stable antibody scFv fragment can tolerate the loss of both disulfide bonds and fold correctly, FEBS Lett. May 15; 427(3):357-61.

In connection with the invention, scFv can be prepared from synthetic or isolated DNA, for example by starting from the actual DNA sequence of the desired scFv. An artificial gene using oligonucleotides is designed, assembled in vitro, and cloned into a suitable expression vector followed by expression in E. coli and purification of the expressed scFv. Alternatively, scFv are manufactured from monoclonal cell lines. For example, a monoclonal cell line is provided, and mRNA from the line is cloned to create a cDNA vector from which the variable heavy (V_{H}) and light (V_{L}) chains are then subcloned into an expression vector.

Other methods for production of antibody fragments are described in current editions in the series of Current Protocols titles (all generally published by John Wiley and Sons, New York), e.g. Current Protocols in Molecular Biology (edited by Frederick M. Ausubel et al., 1991-2004, New York: Greene Pub. Associates and Wiley-Interscience: J. Wiley); Current Protocols in Immunology (edited by John E. Coligan, et al., New York: John Wiley and Sons, 1994-1998).

BRE and SRE that are Fab and Fab′ in the multivalent ligands herein can include Fabs and Fab′ that have specificity for:

various eukaryotes and prokaryotes (including protozoa (amoeba, etc.) fungi, bacteria, and viruses), including eukaryotic and prokaryotic pathogens (animal, including human pathogens);

various animal (including mammalian and human) cells, including among others erythrocytes, immune cells (B-cells, T-cells, dendritic cells, natural killer cells, macrophages, monocytes, neutrophils, eosinophils, etc.), hematopoietic cells, stem cells (embryonic stem cells, stem cells, etc.) and cancer cells;

various antigens, including allergens, and epitopes.

Multivalent ligands that have BRE and SRE that are Fab or Fab′ include those having one or more Fab or Fab′ (having the same or different specificities) alone or in combination with BRE and/or SRE that are not Fab or Fab; those having two or more Fab or Fab′ having different specificity for the same species (e.g., the same cell-type, same biological particle, same pathogen, same antigen, same allergen); those having two or more Fab or Fab′ with the same specificity for a cell-type (e.g., erythrocytes) and two or more Fab or Fab′ having the same or different specificity for the same pathogen; those having two of more Fab or Fab′ having the same of different specificity for the same type of cancer cell; those having one or more Fab or Fab′ with the same specificity for a cell-type (e.g., erythrocytes) and one or more Fab or Fab′ having the same or different specificity for the same cancer cell; those having one or more Fab or Fab′ having the same or different specificity for the same antigen and those having one or more Fab or Fab′ with the same specificity for a cell-type (e.g., erythrocytes) and one or more Fab or Fab′ having the same or different specificity for the same antigen. In specific embodiments, multivalent ligand containing two or more (three, four, etc.) different Fab or Fab′ are useful in pathogen clearance, antigen clearance and in cancer cell control or clearance.

Multivalent ligands of the invention also include those that have BRE and SRE comprising scFv or scFv-hybrid molecules comprising an scFV, for example Fab-scFv, Fab′-scFv, Fab-(scFv)(2), or Fab′-(scFv)(2). Analogous to the immediately preceding paragraph, multivalent ligands are prepared with molecules having various specificities and combinations of specificities.

Therefore multivalent ligands that have BRE and SRE that are scFv or scFv-hybrid include those having one or more scFv or scFv-hybrid (having the same or different specificities) alone or in combination with BRE and/or SRE that are not scFv or scFv-hybrid; those having two or more scFv or scFv-hybrid having different specificity for the same species (e.g., the same cell-type, same biological particle, same pathogen, same antigen, same allergen); those having two or more scFv or scFv-hybrid with the same specificity for a cell-type (e.g., erythrocytes) and two or more scFv or scFv-hybrid having the same or different specificity for the same pathogen; those having two of more scFv or scFv-hybrid having the same of different specificity for the same type of cancer cell; those having one or more scFv or scFv-hybrid with the same specificity for a cell-type (e.g., erythrocytes) and one or more scFv or scFv-hybrid having the same or different specificity for the same cancer cell; those having one or more scFv or scFv-hybrid having the same or different specificity for the same antigen and those having one or more scFv or scFv-hybrid with the same specificity for a cell-type (e.g., erythrocytes) and one or more scFv or scFv-hybrid having the same or different specificity for the same antigen. In specific embodiments, multivalent ligand containing two or more (three, four, etc.) different scFvs or scFv-hybrids are useful in pathogen clearance, antigen clearance and in cancer cell clearance.

Although the preceding two paragraphs exemplify examples with Fab and Fab′ and scFv and scFv-hybrid molecules, respectively, multivalent ligands can be prepared using various combinations of any of Fab, Fab′, scFv, and scFv-hybrid molecules.

A lectin is any of a large group of hemagglutinating proteins found principally in plant seeds. Certain lectins cause agglutination of erythrocytes of certain blood groups; others stimulate the proliferation of lymphocytes.

The term “biological system” is used generally herein to refer to any in vivo or in vitro (including herein ex vivo) system containing signal transduction elements, e.g., signal receptors and biochemical/biological elements for generating a response. A biological system typically contains at least one cell within any environment with which it interacts. A biological system in the context of the uses of multivalent ligands of this invention must contain at least one receptor which can interact with the ligand. In most applications of multivalent ligands, the biological system must contain at least one cell which can respond to the ligand. The response of a cell to the ligand occurs within the biological system and as noted above may be an intracellular response, an intercellular response or both. The biological system can, for example, be a cell in a tissue, a cell in an organ or organism, a cell in a mixture of cells, a cell in a tissue culture, a cell in a tissue or biological fluid sample, and can include biological systems in vivo and in vitro.

“Functional elements (FE)” are chemical or biochemical species (molecules, groups, moieties, etc.) that exhibit some biological or chemical function different from an RE (BRE or SRE). FE can, for example, provide reactive groups or latent reactive groups for attaching another chemical or biological group to a multivalent ligand. For example, an FE can be used to attach a multivalent ligand to a solid surface which may be useful for ligand purification or in applications to analytical or diagnostic assays. FE can be various detectable labels or reporter groups including fluorescent labels, radiolabels and high density labels such as gold particles bound to ligands (e.g., streptavidin labeled with gold particles). Multivalent ligands incorporating detectable labels or reporter groups can be used, for example, in various analytical or diagnostic assays. Of particular interest are multivalent ligands of this invention that are useful in visualization assays, e.g., for the detection of biological particles or molecules in microscopy applications. FE can also exhibit various biological functions, e.g., enzymatic function, ligand-binding function, etc., which may facilitate or enhance a selected application of a multivalent ligand.

Attachment of RE, BRE, SRE and/or FE can be facilitated by use of linker groups intervening between the molecular scaffold of the multivalent ligand and the signal group. Linker groups can be linear or branched, rigid or flexible, hydrophilic or hydrophobic as desired. One of ordinary skill in the art can select linkers from a variety of chemical species suitable for a given application. Further, one of ordinary skill in the art in view of methods and materials that are well known in the art can readily prepare multivalent ligands with linkers having desirable properties.

Multivalent ligands of this invention can be used to modulate signal transduction in prokaryotic and eukaryotic organisms. The ligands function in a variety of signal transduction processes. Prokaryotes have a highly conserved intracellular signal transduction system, the two component system. The major components of this system are varying numbers of alternating histidine-aspartic acid kinase-mediated phosphorylation events, such as virulence, antibiotic resistance, response to environmental stress and sensing. The components of the two component system are highly conserved in prokaryotes.

In contrast, eukaryotes appear to have very few two component systems for signal transduction. This orthogonality makes the two component signaling pathway a prime target for exploitation in therapeutic design for the control of bacterial infection. Major signal transduction systems in eukaryotes are mediated by G-protein-linked receptors and enzyme-linked receptors (including receptor guanylyl cyclases, receptor tyrosine kinases, tyrosine-kinase-associated receptors, receptor tyrosine phosophatases, and receptor serine/threonine kinases). The ability to modulate or regulate signal transduction in these pathways allows control over a wide variety of biological processes in eukaryotic cells and eukaryotic organisms (including mammals and specifically humans) and provides significant opportunity for the design of therapeutics.

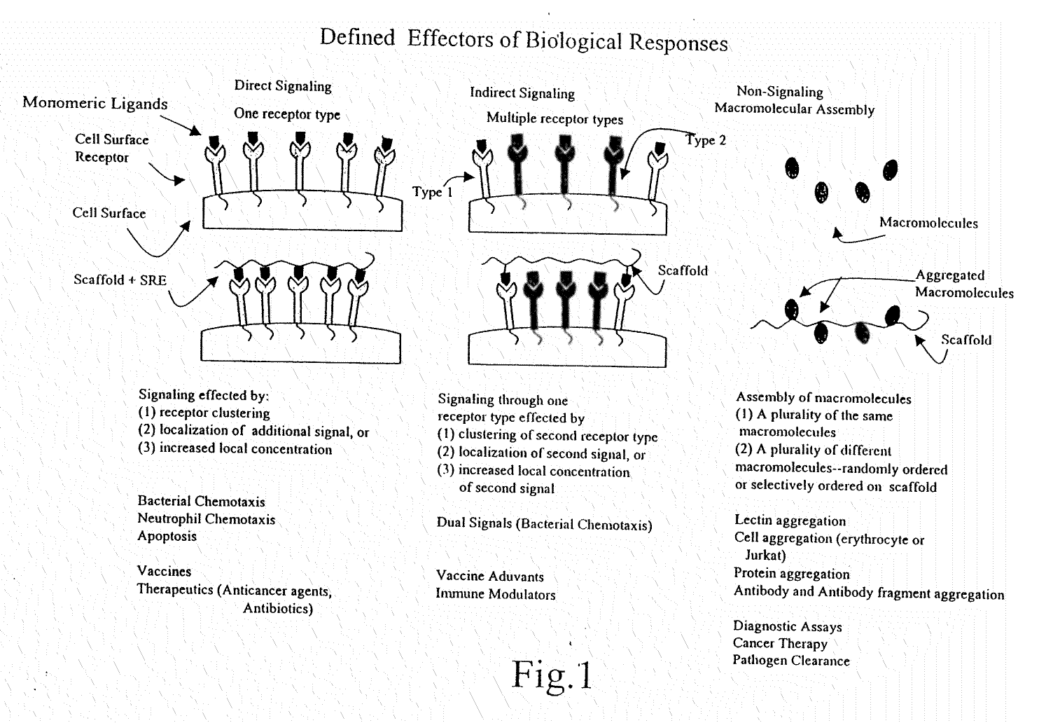

FIG. 1 illustrates several mechanisms by which multivalent ligands of this invention can function as effectors of biological response. A multivalent ligand can be involved directly in signaling where SREs on the multivalent ligand bind to cell surface receptors, similar to monomeric ligands, and directly induce (or inhibit) a response. Use of a multivalent ligand of this invention with SRE attached to a molecular scaffold can facilitate receptor clustering or relocalization on the cell surface, localization of second messengers or simply generally increase the affinity by local increase in SRE (ligand) concentration. Multivalent ligands functioning through direct signaling can be employed in a variety of applications, including those based on disruption of biofilm formation or disruption of cell migration, are of particular interest for vaccines, and other therapeutics (cancer treatment and antibiotics).

Multivalent ligands of this invention can also be involved indirectly in signaling (see FIG. 1) affecting the response of a cell to another signal or ligand. Multivalent ligands may function to sensitize or prime cells for enhanced response to another ligand. Indirect signaling effects may be mediated by clustering or reorganization of one type of cell surface receptor which effectively results in the localization or reorganization of other types of cell surface receptors. Multivalent ligands functioning through indirect signaling can also be useful in a variety of applications, particularly those based on enhancement of a biological response, and are of particular interest for vaccines adjuvants and modulators of immune responses. In direct signaling processes, co-receptors of receptors which bind to multivalent ligand may be, but need not be occupied with a ligand to show an effect on a biological response. In particular instances, multivalent ligands can be functionalize to bind to two different co-receptors to affect the response of one (or both) of the receptors.

Multivalent ligands of this invention also have application simply in binding to or targeting of cells. A multivalent ligand containing at least one recognition element for binding to a cell surface receptor (RE) and containing a functional element (FE) targets the cell with that FE. If FE is a label or reporter group, the multivalent ligand acts to label the cell. If FE has a biological function, the multivalent ligand targets the cell with that function.

Multivalent ligands that contain a plurality of RE (BRE or SRE or both) can function in macromolecular assembly which need not involve any biological signaling function. In such applications, the multivalent ligand need not contain any SRE, the multivalent ligand need only contain more than one recognition element for binding to a cell surface receptor (a recognition element, RE) and preferably a plurality of REs. In such applications, the multivalent ligands directly or indirectly bind to more than one cell resulting in cell aggregation. Cell aggregation may itself trigger a biological response (e.g., the release of signal molecules by a cell), but need not. Multivalent ligands can indirectly cause cell aggregation by binding to a plurality of biochemical species, such as lectins (e.g., Concanavalin A) which in turn bind to cells resulting in cell aggregation. The effect of a multivalent ligand on indirect cell aggregation will be dependent upon the valency of the ligand and on the relative concentrations of the multivalent ligand to the species that causes cell aggregation. At higher concentrations of multivalent ligands with higher valency, binding sites on the species that causes cell aggregation may be saturated inhibiting cell aggregation. At lower concentrations of multivalent ligand, free binding sites will remain and cell aggregation can occur and can be enhanced by the multivalent ligand. Thus, multivalent ligands of this invention can be selectively designed to inhibit or to facilitate cell aggregation. Multivalent ligands functioning for macromolecular assembly can be useful in a variety of applications, particularly those based on cell aggregation, including, but not limited to diagnostic assays, cancer therapy, and pathogen clearance.

The reorganization of receptors on cell surfaces is involved in many important biological reactions including cell migration, adhesion, and the formation of cell to cell junctions. Multivalent ligands of this invention and in particular those ligands which can span the distance between receptors, as discussed above, can be used to reorganize receptors and to modulate response due to the individual signal interactions with the receptors. Reorganization of receptors on the cell surface includes without limitation: changing the relative positions of different cell receptors on the surface, lateral movement of receptors on the surface, the localization of receptors to different sites on the cell surface, changes in the proximity of signal transduction machinery associated with receptors, changes in the proximity of features of the intracellular matrix associated with receptors, changes in the proximity of receptors, clustering of receptors, changes in conformation of receptors, and initiation of receptor-receptor interactions.

In specific embodiments, linear multivalent ligands of this invention are prepared by ring opening metathesis polymerization (ROMP), see for example (54). This method has been used to prepare multivalent inhibitors of cell functions (Sanders, W. J., Katsumoto, T. R., Bertozzi, C. R., Rosen, S. D., Kiessling, L. L. (1996) L-selectin-carbohydrate interactions: Relevant modifications of the Lewis x trisaccharide, Biochemistry 35, 14862-14867; Gordon, E. J., Sanders, W. J., Kiessling, L. L. (1998) Synthetic ligands point to cell surface strategies. Nature 392, 30-31). The ROMP methods have been described in more detail in U.S. Pat. No. 5,587,442 relating to multivalent ligands that are polyglycomers. Improvement of ROMP methods for generating block polymers (and oligomers) and for introducing end-groups on ROMP polymers (and oligomers) have been described in U.S. Pat. Nos. 6,221,315 and 6,291,616 and published international application WO00/78821. (These patent documents are incorporated by reference herein in their entirety particularly for the description of ROMP methods). Choi and Grubbs (2003) Angewandte Chem. Int'l Ed., 42(8):1743 report improved methods for synthesis of ROMP polymers that are useful in the synthesis of multivalent ligands herein. This reference is incorporated by reference herein it its entirety for methods of producing ROMP polymers. Scheme 6 illustrates exemplary methods for modification of ROMP backbones, which can be applied in combination with synthetic methods described in the above listed patents and patent applications to synthesize multivalent ligands of this invention. Scheme 6 illustrates a diimide reduction (Kanai et al., 1997; Hunig, S., Muller, H. R., Their, W. Angew. Chem. Int. Ed. Engl. (1965) 4:271-382; Hart, D. J.; Hong, W.-P.; Hsu, L-Y. J. Org. Chem. (1987) 52: 4665-4673) which can be employed to reduce double bonds in ROMP scaffold backbones. Scheme 6 also illustrates the substitution of ROMP scaffold backbones with OH groups using OsO₄ catalyzed dihydroxylation (Johnson, R. A., Sharpless, K. B. (1993) In Catalytic Asymmetric Synthesis, Ojima, I. (ed.) VCH: New York; Walsh, P. J., Bennani, Y. L., Sharpless, K. B. (1993) Tetrahedron Lett. 34:5545-5548). Those of ordinary skill in the art can prepare multivalent ligands of this invention, particularly those specifically exemplified in formulas herein, employing the descriptions herein and methods that are well known in the art.

Multivalent ligands of this invention prepared by ROMP are exemplified by the general structure:

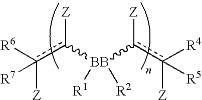

wherein:

n is an integer that is 2 or more and represents the number of repeating units in parentheses that are in the ligand;

the dashed lines indicate optional double bonds;

“BB” represents the backbone repeating unit, which may be cyclic or acyclic, and may be the same or different in a random or block arrangement where the wavy lines indicate that the BB repeating unit can be in a cis or trans configuration in the backbone;

R¹ and R², can be H, an organic group, an FE group or the groups: -L-RE- (L-BRE or L-SRE-), wherein FE is a functional element other than an BRE or an SRE, L represents an optional linker group, RE is a recognition element, and SRE is a signal recognition element;

R⁴ and R⁵ are H, or an organic group;

R⁶ and R⁷ are H, an organic group or an end-group;

Z, independently of other Z in the polymer, is H, OH, OR⁸, SH, a halide (F, Br, Cl, I), NH₂ or N(R⁸)₂ where R⁸ is H or an organic group or Z is absent when there is a double bond at the carbon to which A is attached. R⁴-R⁷ can also be metal chelates.

The integer n is the average number of repeating units in the polymer. Typically n can range up to about 10,000, but there is no practical limit. Preferably the number of repeating units in the multivalent ligands of this invention is defined and can range generally from 2 up to several hundred or several thousand. Preferred multivalent ligands will have n that ranges from 10 to about 500. Multivalent ligands of this invention also include those in which n ranges from 10 to about 25, in which n is 25 or more and those in which n is 50 or more. ROMP can provide polymers of varying average lengths (i.e., varying degree of polymerization, DP) depending on the monomer to ROMP catalyst (i.e., initiator) ratios. The length of all polymers referred to herein (i.e., n or n+m, below) is the length predicted by the monomer to initiator ratios used in the polymerization reaction.

BB can be alkyl, cycloalkyl, cycloalkenyl, and one or more CH₂ groups in the BB moiety can be replaced with —O—, —S—, —NR⁹—, or —CO—, where R⁹ is H or an organic group. Preferred BB have 10 or fewer carbon atoms. Exemplary BB repeating units include among others:

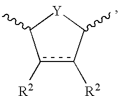

where Y can be —O—, —S—, —NR⁹—, or —CH₂— and there is an optional double bond indicated by the dashed bond;
(CH₂)q, where q is 1 to about 10; and
(CH₂)p—NH—CO—(CH₂)ₘ, where p is 0 to about 10 and m is 0 to about 10.

RE is a recognition element as discussed above that can be any of a variety of chemical or biochemical species that are recognized by and which selectively bind to cell receptors, particularly, transmembrane receptors and cell surface receptors. BRE is a binding recognition element, as discussed above, that includes chemical or biological molecules or fragments thereof that function for binding. SRE is a signal recognition element as discussed above that can be any of a variety of chemical or biochemical species that are recognized by one or more cells and which induce a biological response by the cell; “L” is an optional linker group that can provide functional groups for covalent bonding of the RE, SRE or FE to the polymer (oligomer) backbone. FE is a chemical or biochemical functional group other than an SRE, as discussed above. Other examples of ROMP scaffolds are illustrated in Schemes 2 and 3.

The multivalent ligand of the above formula contains up to n RE (BRE or SRE or both. In specific embodiments all of the monomers carry an RE (BRE or SRE (the number of BRE+SRE is n). In other specific embodiments, regions of spacer monomers that do not carry RE intervene between regions of monomers that carry RE. The BRE and SRE attached to different monomers may be the same or different. In one embodiment, BRE or SRE throughout the multivalent ligand are all the same. In another embodiment, the multivalent ligand contains more than one type of BRE or SRE. In a specific embodiment, the multivalent ligand contains two different types of BRE or SRE or an BRE and an SRE. In this embodiment, the BRE and SRE are non-randomly positioned in the ligand. In another embodiment, the ratios of BRE and SRE are controlled. Preferably monomers carrying the same BRE or SRE are grouped into blocks (as in block polymers) within the multivalent ligand and spacer monomers are optionally positioned between blocks. In other embodiments, R¹ and R² together can form an BRE or SRE.

BRE and SRE are attached to the polymer (oligomer) backbone such that they substantially retain their function for binding or as signals, respectively. For a given BRE or SRE there may be several ways in which it can be bonded into the multivalent ligand, each of which may result in BRE that are different in binding affinity or SRE that are different either in binding affinity or in the level or type of response induced. For example, a peptide signal may be bonding through its N-terminus, through its C-terminus or via an amino acid side group, such as through a lysine side group. The site of attachment of an BRE or SRE to the multivalent ligand is preferably selected to minimize loss of binding function (BRE) or to minimize loss of signal function (SRE) or alternatively the site of attachment may be selected to maximize signal function (SRE). A BRE or SRE may nevertheless exhibit properties that are different from free ligands or free signals (e.g., the binding affinity of an SRE for a cell receptor may be different from that of free signal from which is was derived or which it mimics), but which do not destroy the function of an BRE as a ligand or an SRE as a signal. BRE can include a variety of known cell receptor ligands and in particular can include lectins. BRE can include metal-binding groups, through which binding to metal-binding proteins and peptides is facilitated. SRE can specifically include monosaccharides (e.g., glucose, galactose), disaccharides, polysaccharides (greater than 2 sugar residues), derivatized saccharides (e.g., acylated, sialylated), peptides, derivatized peptides (e.g., N-formyl peptides), peptoids, various chemoattractants, various small drug-like compounds and various epitopes. Note that a particular chemical or biological species may function as an RE with one type or kind of cell and as an SRE with another type or kind of cell.

The linker can provide for spacing of the Res (BRE and/or SRE) or FE group(s) from the backbone of the polymer or can provide for structural flexibility. Linkers may be the same or different on different monomers in the polymer. Linkers that are used in a monomeric scaffold to bond to BRE, SRE or FE can also be all the same or different. In a given multivalent ligand carrying one type of RE group, the linker is preferably the same throughout the polymer. Linkers are generally selected so that they are compatible with the intended application of the multivalent ligand and to avoid interference with the function of signal groups. The linker is preferably linear and preferably ranges in length from 1 to about 20 atoms, but can be significantly longer, from 2-100 atoms or even more than 100. The linker may contain alicyclic groups (such as a cyclohexyl group). The linker can be an alkyl chain carrying functional groups for bonding to the backbone of the ligand and to the signal. The linker can also be an ether, ester, ketone, amine, amide or thioether chain. In a specific embodiment, the linker can be described as an linear alkyl chain having from 1 to about 20 carbon atoms in length in which one or more non-neighboring CH₂ groups are optionally replaced with an —O—, —S—, —NH—, NR¹⁰—, —CO—, —NH—CO—, —O—CO—, —HC≡CH—, or —C≡C— group, where R¹⁰ is an alkyl or aryl group. Linker CH₂ groups can be substituted with halogens, alkoxy, or alkyl groups. In the absence of a linker group, the ROMP backbone or the signal group itself must provide the functionality for covalent bonding of the signal to the backbone. Exemplary linkers include those illustrated in Scheme 3.

R¹, R², R⁴, R⁵, R⁶, R⁷, R⁸ and R⁹ can be organic groups. Organic groups include, without limitation, alkyl groups, alkenyl groups, and aryl groups as well as substituted alkyl, alkenyl and aryl groups. Substituents for alkyl, alkenyl and aryl groups include halogens (F, Cl, Br, I), —CN, —NO₂, —OH, —SH, —NH₂, —N(R¹⁰)₂, —SR¹⁰ and OR¹⁰, where R¹⁰ is an alkyl or aryl group. Aryl groups may also contain alkyl or alkenyl substituents. Organic groups will typically have from 1 to about 20 carbon atoms, and preferably have 1 to about 10 carbon atoms. Alkyl groups may be straight-chain, branched or cyclic (or contain portions that are cyclic). One or more non-neighboring —CH₂— groups in an alkyl or alkenyl group can be replaced with —O—, —S—, —NH—, —NR¹⁰—, where R¹⁰ is an alkyl or aryl group.

R⁶ and R⁷ can be end-groups, such as those described in U.S. Pat. No. 6,291,616, which is incorporated in its entirety herein for description of methods of synthesis of multivalent ligands having end-groups using ROMP methods. End-groups can include a latent reactive group or a non-reactive functional group as described in the cited patent application. The presence of a latent reactive group would allow for later functionalization of a polymer multivalent ligand at an end-group. End-groups can contain functionality for binding to solid surfaces.

The end-group may itself be a linkage to a solid support material. Latent reactive groups include: azides, a nitro group, a disulfide, a cyano group, an acetal group, a ketal, a carbamate, a thiocyanate, an activated ester, or an activated acid (activated esters and acids are those containing good leaving groups that are activated in particular for nucleophilic attack). Non-reactive end-groups include natural products or analogs thereof (e.g., biotin), metal chelators (e.g., nitrilotriacetic acid), metals (e.g., Zn²⁺), and fluorescent labels (amide derived BODIPYL FL EDA which is 4,4-difluoro-5,7-dimethyl-4-boro-3a,4a-diaza-s-indacene-3-propionyl ethylenediamine). End-groups can include FE.

The multivalent ligand optionally contains one or more functional elements that are not SRE or BRE. Preferred multivalent ligands contain significantly fewer FE compared to SRE or BRE. FE can be or contain any of the reactive or non-reactive groups listed above or described in U.S. Pat. No. 6,291,616 as “end-groups”. FE can also have enzymatic or other protein function.

When prepared by the ROMP methods, such as those described in U.S. Pat. Nos. 6,271,315 and 6,291,616 (which are incorporated by reference herein in their entirety for methods of synthesis of multivalent ligands), R⁴ and R⁵ are derived from the metal carbene catalyts, i.e., they are substituents on the metal carbene carbon of the metal carbene catalyst and in specific embodiments are H and a phenyl group. When using ROMP, R⁶ and R⁷ are typically derived from the capping agent, i.e., are the substituents on the electron-rich alkene capping agent, such as hydrogen in the case of ethyl vinyl ether.

In a specific embodiment multivalent ligands of this invention include those of formula:

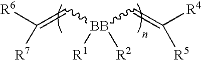

wherein BB, R¹⁻², and R⁴⁻⁷ are as defined above. In specific embodiments, one of R¹ or R² is H and the other is L-RE. In specific embodiments, one or R¹ or R² is H and the other is L-SRE. In specific embodiments, one or R¹ or R² is H and the other is L-BRE. In specific embodiments, RE is a lectin or a cell receptor ligand that is comprised within a lectin. In specific embodiments, SRE is a monosaccharide, a disaccharide or a relatively short saccharide having up to about 10 sugar residues. In other specific embodiments, SRE is a peptide or a derivatized peptide (e.g., an N-formyl peptide).

In another specific embodiment the invention relates to multivalent ligands of the formula:

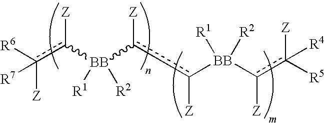

wherein the dashed line indicates an optional double bond and wherein Y, independently of Y in other monomers, R¹⁻², independent of R¹⁻² in other monomers, and R⁴⁻⁷ are as defined above. In specific embodiments, Y is —CH₂—. In specific embodiments, one of R¹ or R² is H and the other of R¹ or R² is -L-RE. In specific embodiments, one of R¹ or R² is H and the other of R¹ or R² is -L-SRE. In specific embodiments, one of R¹ or R² is H and the other of R¹ or R² is -L-BRE. R¹ and R² together may form an -L-BRE or -L-SRE. In yet other specific embodiments, SRE is a peptide or derivatized peptide. In yet other specific embodiments, SRE or BRE is an antibody, or antibody fragment (e.g., Fab′). In yet other specific embodiments, SRE or BRE is an antigen, or epitope. In other specific embodiments, BRE is a metal-binding ligand. When no double bond is present the ring carbons typically carry addition hydrogens, but may be substituted with other groups, such as alkyl groups having 1-6 carbon atoms or halides, that do not interfere with the function of any R¹ or R² group.

In yet another specific embodiment the invention relates to multivalent ligands of the formulas:

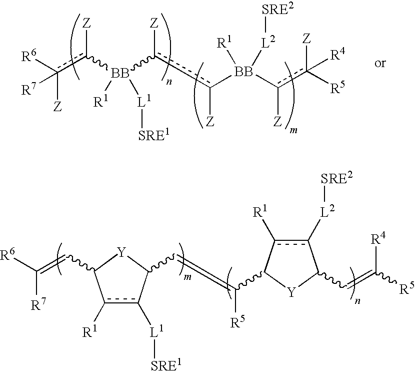

in which m is the number of monomers carrying a first SRE (SRE¹) and n is the number of monomers carrying a second SRE (SRE²). L¹ and L² are linkers as described above which may be the same or different. All other variables are as defined in earlier formulas and dashed lines indicating optional double bonds. Both m and n are integers that can range most generally from 1 up to about 10,000, but which more typically will range from 1 to several hundred or several thousand. The value of m may be the same as or different from that of n. In preferred ligands, n+m ranges from 5 or more up to about 300. Multivalent ligands of this invention include those in which n+m ranges between about 10 and 25, those in which n+m is 25 or more, those in which n+m is 50 or more, those in which n+m is 100 or more, and those in which n+m is 300 or more. Exemplary multivalent ligands of this invention include those of the above formulas wherein one or more of the SRE are replaced with BRE. These exemplary multivalent ligands can contain one or more different SRE in combination with one or more different BRE. These exemplary multivalent ligands can contain one or more different BRE.

Other exemplary multivalent ligands include those of the formulas:

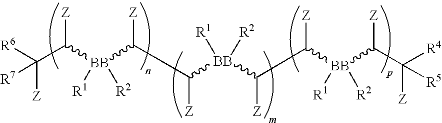

wherein n, m and p are integers with a value greater than 3 and other variables are as defined above; and

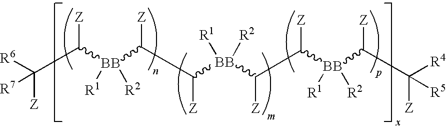

wherein n, m, p and x are integers each of which has a value greater than 1 and all other variables are as defined above. Multivalent ligands of these formulas can contain multiple blocks of monomer regions having the same BRE or SRE. Multivalent ligands of these formulas can contain multiple blocks of monomer regions one BRE or SRE and multiple blocks of monomer regions containing another BRE or SRE. Multivalent ligands of these formulas can also contain multiple blocks of monomer regions carrying BRE or SRE with intervening spacer regions that carry no RE.

In specific embodiments, multivalent ligands of this invention include those having about 50 to about 1000 polymer repeating units, those having 50 to about 500 polymer repeating units, those having 50 to about 200 polymer those having 100 to about 200 polymer repeating units, those having 100 to about 250 polymer repeating units or those having 100 to about 200 polymer repeating units.

In specific embodiments, ATRP polymeric multivalent ligands of this invention include those containing at least two to about 1000 RE, those containing two to about 500 RE, those containing two to about 250 RE, those containing two to about 100 RE, those containing two to about 50 RE, those containing ten to about 50 RE, those containing ten to about 20 RE of those containing 5-15 RE. The density of RE in a given polymeric multivalent ligand can be adjusted for a given application. The spacing or average spacing between RE within a given polymeric multivalent ligand can be adjusted for a given application. FE can be added to polymeric ligands and positioned or spaced therein as desired for a given application. The chemical properties of the polymer can also be adjusted by selection of chemical groups other than RE's bonded to the polymer backbone. For example, the polymer can be made more or less hydrophilic or the solubility of the polymer can be adjusted as desired by introduction of functional groups.

The invention also provides libraries of ATRP and/or ROMP polymer multivalent ligands which contain multivalent ligands of varying polymer length, ligand type, ligand density, ligand spacing or mixtures of ligands which are useful for the selection of multivalent ligands exhibiting a desired biological or chemical activity. Multivalent ligand libraries can be generated to contain members which span a given structural, or chemical variation to allow selection of a given ligand property or activity or which allow selection of a ligand having an enhanced property or activity.

In specific embodiments, this invention provides multivalent ligands that are derivatized linear polymers made by atom-transfer radical polymerization having the formula:

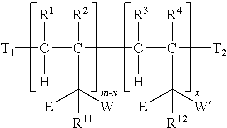

where the structure reflects the number of repeating units, but does not necessarily reflect the relative positions of the repeating units;

m and x are integers and m is the number of monomers in the polymer;

W and W′ are groups independently selected from -L-BRE, -L-FE, -L-SRE, a hydrogen or an organic group;

E is an electron withdrawing group, which may be connected in the structure by a single or a double bond (if E is bonded through a double bond, then R¹¹ and/or R¹² are absent);

L is an optional linker group;

T₁₋₂ are polymer end groups which can include, among others, reactive or non-reactive groups and latent reactive groups; and

R¹⁻⁴ can be the same or different groups and are most generally, independently of one another, hydrogen or any organic groups;

R¹¹⁻¹² can be the same or different groups and are most generally, independently of one another, hydrogen or any organic groups, R¹¹ and/or R¹² being absent, if E is bonded through a double bond; and

where the polymeric ligand contains at least one and preferably more than one W or W′ that is a RE (a BRE and/or an SRE group).

In specific embodiments, E can be O═ (to form a carbonyl group), —CN, —SO₂R⁵, or —SOR⁵, where R⁵ is a hydrogen or any organic groups, including hydrocarbyl groups and substituted hydrocarbyl groups.

Preferred BRE or SRE groups include those that function for aggregation of biological species, e.g., antibodies or portions thereof, or function for protein oligomerization, e.g., BRE or SRE that function for binding to proteins of peptides, such as BRE or SRE that chelate to metals or that bind to His-tags. Preferred RE of this invention are antibody fragments, including Fab and Fab′ fragments having a selected specificity and groups having metal chelating function.

The length of a polymer and the number of RE and/or FE in a given ligand can be adjusted for a given application. Of particular interest are multivalent ligands of the above formula in which m ranges from about 50 to about 200, those in which m ranges from about 100-200, those in which m ranges from about 140-160 and those in which m is about 150. Also of particular interest are multivalent ligands which contain two or more RE, those which contain 0.5 or more RE, those which contain 10 or more RE, those which contain from 2 to about 20 RE, those which contain from 5 to about 20 RE and those which contain from 10 to about 20 RE.

More particularly R¹⁻⁴ can be hydrogen or any hydrocarbyl groups, including hydrocarbyl groups substituted with one or more heteroatoms (e.g., N or O), one or more halogens, one or more —SR⁵ groups, one or more —O—R⁵ groups (where R⁵ is a hydrogen or any organic groups, including hydrocarbyl groups and substituted hydrocarbyl groups), one or more amine groups (—N(R⁵)₂ (where R⁵, independent of other R⁵ groups is a hydrogen, or any organic groups again including any hydrocarbyl or substituted hydrocarbyl groups), or one or more halogen groups.

In specific embodiments, this invention provides multivalent ligands that are derivatized linear polymers made by atom-transfer radical polymerization having the formula:

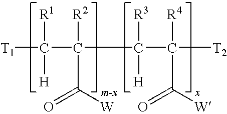

where variables are as defined above.

In more specific embodiments, the invention provides derivatized linear polymers having the formula:

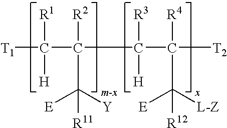

where:

E and R¹¹ and R¹² are as defined above;

m and x are integers, x is the number of monomers carrying a Z group and m is the number of monomers in the polymer; the structure of the above formula reflects the relative number, but does not reflect the relative positions of Y and Z groups in the polymer;

Z is a metal chelating group or a metal chelating group chelated to one or more metal species;

Y is a chemical group that is not a metal chelating group, which more specifically can be selected from any organic group, an -L₂-BRE group, an -L₃-FE group, or an -L₁- SRE group (other than a metal chelating group);

T₁₋₂ are polymer end groups which can include, among others, reactive or non-reactive groups, latent reactive groups, groups for bonding to solid or a bond, with optional linker to a solid; and

L and L¹⁻³ are optional linker groups; and

R¹⁻⁴ can be the same or different groups and are most generally, independently of one another, hydrogen or any organic groups, or more particularly hydrogen or any hydrocarbyl groups, as well as hydrocarbyl groups substituted with one or more heteroatoms (e.g., N or O), one or more halogens, one or more —SR⁵ groups, one or more —OR⁵ groups (where R⁵ is a hydrogen or any organic groups, including hydrocarbyl groups and substituted hydrocarbyl groups), one or more amine groups —N(R⁵)₂ (where R⁵, independent of other R⁵ groups, is a hydrogen, or any organic groups again including any hydrocarbyl or substituted hydrocarbyl groups), or one or more halogen groups. Organic groups can also include —OR⁵ or —N(R⁵)₂ groups, where R⁵ can be selected from hydrogen, alkyl, aryl, or substituted alkyl or aryl groups, in particular R⁵ can include halogen or OH substituted alkyl or aryl groups.

More specifically R¹⁻⁴ can be selected, independently of other R¹⁻⁴ groups, from the group consisting of hydrogen, alkyl groups, alkenyl groups, aryl groups, alkyl- or alkenyl-substituted aryl groups, halogen-substituted aryl groups, amine-substituted aryl groups, aryl-substituted alkyl or alkenyl groups, halogen-substituted alkyl or alkenyl groups, amine-substituted alkyl or alkenyl groups and heteroaryl groups (in which at least one of the atoms in a five or six membered ring is a heteroatom, particularly N or O). R¹⁻⁴ groups can also be selected from ether groups (e.g. those containing —CH₂—O—CH₂— linkages), alkanolamine groups, e.g., —NH—(CH₂)_{y}—OH (where y is an integer from 1-10), —NH(CH₂)_{y}—C(OH)—(CH₂)_{z}—OH (where y and z are integers from 1 to 10).

In yet more specific embodiments Y is an —OM group in which M is a hydrogen, an alkyl group or an aryl group (including a phenyl group), a N-succinimidyl group, a halogenated phenyl group, an nitrophenyl group carrying one or more nitro groups, or an imidazole group.

The multivalent ligand may further be bonded to a solid directly or indirectly through a linker group at one of the W, W′, Y or L-Z groups.

In yet more specific embodiments R¹⁻⁴ are hydrogen, alkyl or aryl groups, including phenyl groups. Alkyl, alkenyl, aryl groups include those having from 1 to about 20 carbon atoms. Alkyl groups can include small alkyl groups having from 1 to 6 carbon atoms. Alkyl groups specifically include methyl groups, ethyl groups, propyl groups (of various structures), butyl groups (of various structures), pentyl groups (of various structures) and hexyl groups (of various structures).

In yet more specific embodiments L and L¹⁻³ groups can include amino acid groups or peptide groups including derivatized peptide groups, particularly small peptide groups having from about 2 to about 10 amino acids. L and L¹⁻³ can include N-formyl peptides and N-acetyl peptides for example. The amino acids in an L group may be the same or different and can include those having one or more glycines, or one or more lysines (particularly those having from 1-5 glycines, lysines or both and more particularly those having -Gly-Gly- or -Lys-Lys-. Linker groups can also contain chemical moieties (alkyl or ether chains, for example) that provide for linking to the Z (or SRE, BRE, or FE or other group) or to the polymer.

In specific embodiments Z is a metal-chelating group, particularly a nickel-chelating group. In specific embodiments, Z is a metal-chelating group chelated to a metal, or more specifically a nickel-chelating group chelated to nickel. More specifically Z contains a nitriloacetic acid group to provide nickel-chelating functionality. In specific embodiments -L-Z has the structure:

—NH-(A)ₙ-NH—(CH₂)ₚ—C(CO—OR)—N(CH₂CO—OR)₂

where A is an amino acid and n is an integer representing the number of amino acids linked (where A may be different amino acids), A is preferably glycine or lysine, n is preferably 1-5; p is an integer representing the number of —CH₂— moieties and R is hydrogen, an alkyl group or an aryl group (including a benzyl group). Z can also be a form of the above indicated structure to which nickel or another metal is chelated.

In further specific embodiments L and L¹⁻³ can have the formula:

—NR⁵—(CH₂)_{y}[—O—(CH₂)_{z}—]_{b}—O—(CH₂)ₓ—NH—

where x, y, z and b are integers ranging from 1 to about 10, with x, y and z preferably 2, 3 or 4 and b preferably 1-5.

In yet further specific embodiments L and L¹⁻³ can have the formula:

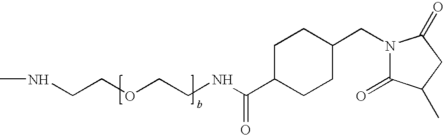

where b is an integer from 1-about 5. In particular, linkers of this type are useful for attachment of Fab′ fragments (via thiol linkages to the maleimide) to the polymer as illustrated in Scheme 13 and Scheme 14.

In specific embodiments, this invention provides multivalent ligands having the formula:

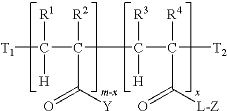

where variables are as defined above.

In other specific embodiments, the invention provides polymers containing reactive FE which can react to conjugate, antibody fragments, including Fab′ fragments. Such reactive FE include, among others, groups comprising a maleimide moiety which can react with thiols. More specifically, FE groups can be linked to the polymer via polyether linkages or amino polyether linkages.

In all of the specific formulas herein for multivalent ligands formed by ATRP technology, BRE, SRE and FE can take all values of BRE, SRE and FE that are specifically defined for ROMP polymers. In all of the specific formulas herein for multivalent ligands formed by ROMP technology, BRE, SRE and FE can take all values of BRE, SRE and FE that are specifically defined for ATRP polymers.

Derivatized linear polymers (including both ROMP and ATRP polymers) of this invention are useful in particular as multivalent ligands in which valency can be optimized for aggregation of biological molecules and particles including proteins, antibodies and fragments thereof, and various cells, as effectors in biological systems or for eliciting a desired biological response.

In specific examples, the invention provides polymeric multivalent ligands which selectively bind to and/or facilitate aggregation of biological pathogens. More specifically, the invention provides multivalent ligands which function for or facilitate pathogen clearance in an animal (including mammals, and specifically, humans) infected with the pathogen. The invention further provides methods for aggregation of pathogens from an animal (including mammals and humans) host using multivalent ligands.

In other specific examples, the invention provides multivalent ligands which chelate with one or more metals, and provides methods for the use of such multivalent ligands, containing metal chelating groups or in which metals are chelated to the metal chelating groups, in analytical, catalytic and therapeutic applications. The invention specifically provides polymeric multivalent ligands containing nickel chelating groups which function for oligomerization of peptides or proteins containing His-tags.

The multivalent ligands of this invention are useful in methods for controlling or modulating the effect of chemical signals in a biological system. Applications of multivalent ligands to bacterial and eukaryotic chemotaxis, to migration of leukocytes (particularly neutrophils), to immune responses of B-cells and T-cells, to cell aggregation, and to signaling of apoptosis are exemplified herein below.

Multivalent ligands of this invention which carry bacterial chemoattractants can be employed to disrupt colonization and biofilm formation by bacteria. Chemotaxis is a virulence factor which facilitates bacterial colonization of its host. Disruption of colonization of host tissue prevents host-bacterial interactions, prevents colonization and inhibits or retards infection. The methods of this invention can be applied specifically to disruption of colonization, for example, by Staphylococus aureus (for treatment of staph infections) and Vibrio cholerae (for treatment of cholera). One bacterial survival mechanism involves the formation of microcommunities with functional heterogeneity (biofilms). Biofilm formation and maintenance are regulated by soluble small molecule-based factors. These factors control signal transduction pathways that allow bacteria to sense their environment and conversions to biofilm formation are mediated by two-component signaling systems. Disruption of biofilm formation renders bacteria more susceptible to host defenses or to antibiotic treatment and can inhibit or retard infection. Multivalent ligands which disrupt biofilm formation can be particularly useful in preventing or treating infections of the lung, for example for treating or preventing lung infection by the opportunistic pathogen Pseudomonas aeruginosa. Infection by this organism is a leading cause of death in patients with cystic fibrosis. Another mechanism for bacterial survival is induction of a virulence response upon increased bacterial cell density. This virulence response is induced by the release of soluble factors when increased cell density is sensed. Disruption of the responses of bacteria to increased cell density by multivalent ligands of this invention can be used to control bacterial virulence, for example, this method is applicable to the control of virulence of Staphylococus aureus.

Multivalent ligands of this invention can in similar ways be employed to disrupt infection by eukaryotic pathogens and parasites, including among others, Trypanosoma cruzi (Chagas disease) Trypanosoma brucei (sleeping sickness), tapeworms, hookworms, and Plasmodium falciparum (malaria).

The multivalent ligands of this invention can be used to modulate immune response toward epitopes and antigens (e.g. by modulating the immunogenicity of these species). For example, multivalent ligands can be designed to stimulate or inhibit leukocyte responses, including migration. Stimulation of such response can be used to enhance recognition of non-self cells for clearance and to treat infection. Multivalent ligands can also be designed to modulate the activation and/or deactivation of B-cells or T-cells in response to chemical signals to improve and enhance desired immune responses. B-cells and T-cells can be treated with multivalent ligands of this invention in vitro, in vivo and ex vivo.

Autoimmune diseases involve aberrant function of a cell signal recognition process in which self cells are incorrectly marked for clearance. Multivalent ligands of this invention which modulate cell responses of immune system cells to epitopes can be employed to inhibit or attenuate autoimmune disorders. In a specific embodiment, ligands carrying self epitopes mistakenly recognized as “non-self” and certain B-cell or T-cell epitopes can be employed in a tolerization process to ameliorate autoimmune responses.

The multivalent ligands of this invention also have application to the treatment of undesired cell proliferation (cancer) and undesired cell migration (metastasis). Cancer cells have distinct surface features (e.g., epitopes) that distinguish them from non-cancer cells. The multivalent ligands of this invention can be designed to promote recognition of cancer-specific epitopes as non-self cells by the immune system such that cancer cells are cleared by the immune system. Multivalent ligands carrying cancer cell epitopes and B-cell or T-cell epitopes can be employed in a sensitization process to promote clearance of the cancer cells. Cancer metastasis is deviant cell migration. The movement, adhesion, and junction formation of cancer cells are mediated, at least in part, by interaction of cancer cells with the multivalent extracellular matrix. Multivalent ligands can be designed to inhibit or prevent movement, adhesion and junction formation and thus inhibit metastasis.

This invention provides pharmaceutical and therapeutic compositions comprising multivalent ligands with BRE and/or SRE groups selected to provide therapeutic benefit in combination with a pharmaceutically acceptable carrier or excipient adapted for use in human or veterinary medicine. The multivalent ligands may be combined with each other to achieve a desired pharmaceutical response or administered in combination with other known drugs or therapeutic agents, including without limitation antibacterial and other antimicrobial agents. The multivalent ligand is present in the pharmaceutical compositions in an amount, or in combination with other ligands in a combined amount, sufficient to obtain the desired therapeutic benefit. The carrier or excipient is selected as is known in the art for compatibility with the desired means of administration, for compatibility with the selected multivalent ligand(s) and to minimize detrimental effects to the patient.

This invention is also directed to pharmaceutically acceptable esters and salts of the multivalent ligands of various formulas and structures described herein. Acid addition salts are prepared by contacting compounds having appropriate basic groups therein with an acid whose anion is generally considered suitable for human or animal consumption. Pharmacologically acceptable acid addition salts include but are not limited to the hydrochloride, hydrobromide, hydroiodide, sulfate, phosphate, acetate, propionate, lactate, maleate, malate, succinate, and tartrate salts. All of these salts can be prepared by conventional means by reacting, for example, the selected acid with the selected basic compound. Base addition salts are analogously prepared by contacting compounds having appropriate acidic groups therein with a base whose cation is generally considered to be suitable for human or animal consumption. Pharmacologically acceptable base addition salts, include but are not limited to ammonium, amine and amide salts.

Pharmaceutically acceptable esters of compounds of this invention are prepared by conventional methods, for example by reaction with selected acids. Pharmaceutically acceptable esters include but are not limited to carboxylic acid esters R^{E}COO-D (where D is a cationic form of a compound of this invention and where R^{E} is H, alkyl or aryl groups).

This invention is also directed to prodrugs of multivalent ligands and derivatives which on being metabolized will result in any of the ligands of this invention. Labile substituents may be protected employing conventional and pharmaceutically acceptable protecting groups removable on metabolism. Pharmaceutically active compounds may be derivatized by conventional methods to provide for extended metabolic half-life, to enhance solubility in a given carrier, to provide for or facilitate slow-release or timed-release or enhance or affect other drug delivery properties.

The multivalent ligands according to the invention may be formulated for oral, buccal, parenteral, topical or rectal administration. In particular, the ligands according to the invention may be formulated for injection or for infusion and may be presented in unit dose form in ampules or in multidose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g. sterile, pyrogen-free water, before use.

The pharmaceutical compositions according to the invention may also contain other active ingredients, such as antimicrobial agents, or preservatives. In general, pharmaceutical compositions of this invention can contain from 0.001-99% (by weight) of one or more of a multivalent ligand described herein.

For administration by injection or infusion, the daily dosage as employed for treatment of an adult human of approximately 70 kg body weight will range from 0.2 mg to 10 mg, preferably 0.5 to 5 mg, which can be administered in 1 to 4 doses, for example, depending on the route of administration and the clinical condition of the patient. These formulations also include formulations in dosage units. This means that the formulations are present in the form of a discrete pharmaceutical unit, for example, as tablets, dragees, capsules, caplets, pills, suppositories or ampules. The active compound content of each unit is a fraction or a multiple of an individual dose. The dosage units can contain, for example, 1, 2, 3 or 4 individual doses for ½, ⅓ or ¼ of an individual dose. An individual dose preferably contains the amount of active compound which is given in one administration and which usually corresponds to a whole, one half, one third or one quarter of a daily dose. The magnitude of a prophylactic or therapeutic dose of a particular multivalent ligand will, of course, vary with the nature of the severity of the condition to be treated, the particular ligand compound and its route of administration. It will also vary according to the age, weight and response of the individual patient.

The therapeutic compounds of the present invention are preferably formulated prior to administration. The present pharmaceutical formulations are prepared by known procedures using well-known and readily available ingredients. In making the compositions of the present invention, the active ingredient will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. The compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient by employing procedures well known in the art.

The compositions are preferably formulated in a unit dosage form, each dosage containing from about 0.5 to about 150 mg, more usually about 0.1 to about 10 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

The invention is further directed to therapeutic methods that comprise the step of administering a pharmaceutical composition of this invention to an individual (animal or human) that can derive therapeutic benefit from the compositions.

Multivalent ligands of this invention can be employed in diagnostic applications for the detection of biolgical molecules and or biological particles in biological systems (including in biological samples or biological fluids obtained from individuals).

Multivalent ligands of this invention can also be employed in non-therapeutic applications, for example, to prevent or inhibit biofouling in a selected environment or to remove undesired cells from a selected environment. Compositions of this invention for use in such non-therapeutic comprise one or more multivalent ligands of this invention in an amount or in a combined amount effective for obtained a desired function, e.g., effective for affecting bacterial or microbial chemotaxis or effective for aggregating cells in a sample or a biological system. Compositions can be formulated using any appropriate solvent or carrier system which may be an aqueous solution, a lyophilized or a spray-dried material so long as desired function is maintained.

E. A. Smith, W. D. Thomas, L. L. Kiessling and R. M. Corn (2003), Surface Plasmon Resonance Imaging Studies of Protein—Carbohydrate Interactions, J. Am. Chem. Soc., 125, 6140-6148; M. C. Schuster, D. A. Mann, T. J. Buchholtz, K. M. Johnson and L. L. Kiessling (2003). Parallel Synthesis of Glycomimetic Libraries that Target a C-Type Lectin, Org. Lett., 5, 1407-1410; and Gestwicki J. E., Cairo C. W., Borrok M. J., Kiessling L. L., Visualization and characterization of receptor clusters by transmission electron microscopy, Methods Enzymol. 2003; 362:301-12 are incorporated by reference herein to the extent not inconsistent with this specification to provide additional details of multivalent ligand synthesis and applications.

The following examples, further illustrate and further describe the invention, but are in no way intended to limit the invention.

### THE EXAMPLES

### Example 1

### Modulation of Bacterial Chemotaxis

The molecular events leading to bacterial chemotaxis have been well studied, and the process has served as a general model for receptor-mediated responses [Parkinson, J. S. (1993) Signal transduction schemes of bacteria, Cell, 73, 857-871; Hazelbauer, G. L., Berg, H. C., Matsumura, P. (1993) Bacterial motility and signal transduction. Cell, 73, 15-22; Alon, U., Surette, M. G., Barkai, N., Leibler, S. (1998) Robustness in bacterial chemotaxis, Nature 393, 18-19; Barkal, N., Leibler, S. (1997) Robustness in simple biochemical networks, Nature 387, 913-917]. During chemotaxis in Escherichia coli, chemoattractants, such as sugars and amino acids, and chemorepellents are recognized by specific receptors at the bacterial plasma membrane [Grebe, T. W. & Stock, J. (1998) Bacterial chemotaxis: The five sensors of a bacterium, Curr. Biol., 8, R154-R157]. For these investigations of multivalent ligand activity, galactose was selected as a model chemoattractant. The related compound, β-methyl galactopyranoside, is also a chemoattractant, indicating that the attachment of substituents at the anomeric position of galactose does not abolish its chemotactic activity [Adler, J., Hazelbauer, G. L., Dahl, M. M. (1973) Chemotaxis towards sugars in Escherichia coli. J. Bacteriol. 115, 824-847]. This observation suggests that galactose residues can be tethered through an anomeric linker to create a multivalent display. For galactose-mediated signaling, the saccharide must bind to the soluble periplasmic glucose/galactose-binding protein (GGBP), which, in turn, interacts with the galactose-sensing chemoreceptor, Trg [Adler et al., 1973; Hazelbauer, G. L., Adler, J. (1971) Role of the galactose binding protein in chemotaxis of Escherichia coli toward galactose, Nature New Biol. 230, 101-104]. Galactose-GGBP binding to Trg initiates a signaling pathway that results in reversal of the direction of flagellar spin, allowing the bacteria to swim towards the nutrient [Parkinson, 1993; Hazelbauer et al., 1993; Silversmith, R. E., Bourret, R. B. (1999) Throwing the switch in bacterial chemotaxis, Trends Microbiol., 7, 16-22].

Bacterial chemotaxis requires an extremely sensitive sensing system with a broad dynamic range. Through their chemoreceptors, bacteria can detect very small changes in ligand concentration over many orders of magnitude [Mesibov, R., Ordal, G. W., Adler, J. (1973) The range of attractant concentrations for bacterial chemotaxis and the threshold and size over this range, J. Gen. Physiol. 62, 203-223; Jasuja, R., Keyoung, J., Reid, G. P., Trentham, D. R., Khan, S. (1999) Chemotactic responses of Escherichia coli to small jumps of photoreleased L-aspartate, Biophys. J. 76, 1706-1719]. A recent mathematical model proposed by Bray et al. to explain this remarkable feature suggests signal transduction is regulated by changes in lateral clustering of the chemoreceptors [Bray, D., Levin, M. D., Morton-Firth, C. J. (1998) Receptor clustering as a cellular mechanism to control sensitivity, Nature 393, 85-88; Levit, M. N., Liu, Y., Stock, J. B. (1998). Stimulus response coupling in bacterial chemotaxis: receptor dimers in signaling arrays. Mol. Microbiol. 30, 459-466; Duke, T. A. J., Bray, D. (1999) Heightened sensitivity of a lattice of membrane receptors, Proc. Natl. Acad. Sci. USA, 96, 10104-10108].

In this model, clusters of bacterial chemoreceptors exchange ligand binding information, such that receptor clusters are more active in signal generation than individual receptors [Bray, et al., 1998; Duke et al., 1999)]. Multivalent ligands of this invention having distinct valencies can differentially reorganize the receptors and thus control lateral receptor organization may result in modulation of the chemotactic response.

Galactose-bearing ligands 1-4 of varying defined valencies were generated using ROMP methods (Scheme 1). The galactose residues in the multivalent ligands are tethered to the molecular scaffold (polymer backbone) via a short linker. The interaction of monomer 1 was at least as favorable as that of galactose in an in vitro binding assay, thus the attachment of the linker did not prevent galactose binding to purified GGBP.

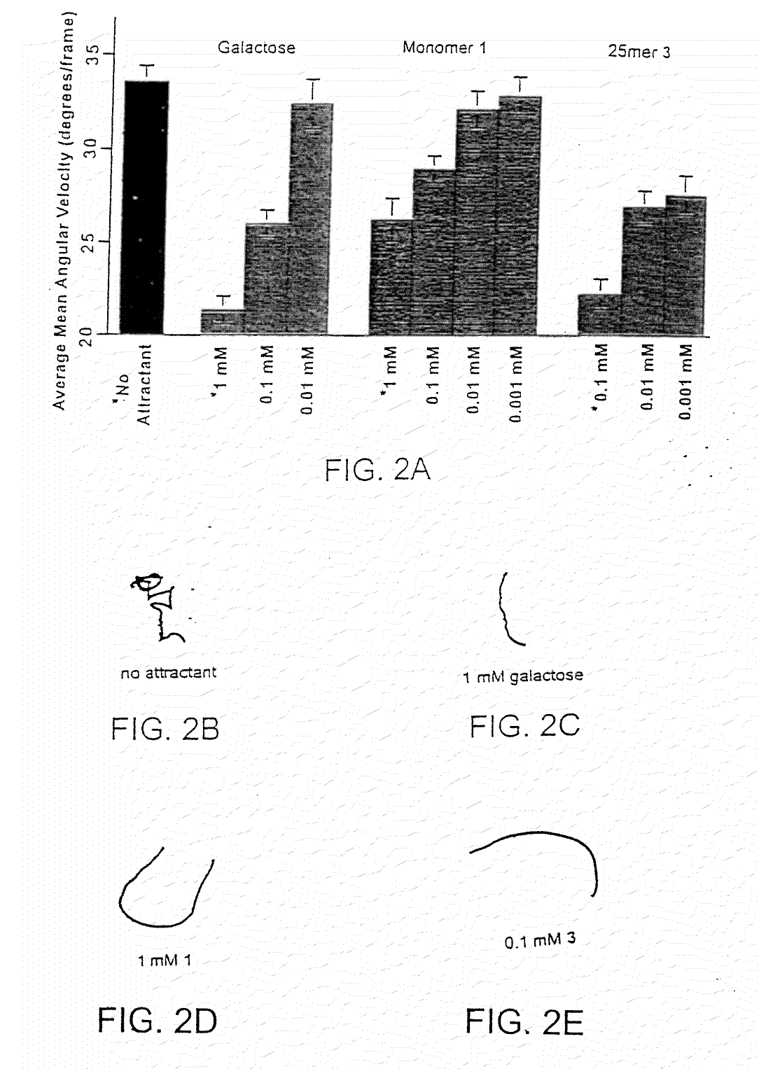

Ligands functionalized with galactose such as monovalent ligand 1 and multivalent ligand 3, also serve as attractants in vivo. This was demonstrated by monitoring the behavioral response of E. coli to these ligands. The locomotion behavior of E. coli occurs in two modes, running and tumbling, which are defined by the direction of the flagellar spin and, ultimately, the signal transduction response that arises from interaction of chemoreceptor with ligand [Berg, H. C., Brown, D. A. (1972) Chemotaxis in Escherichia coli analysed by three dimensional tracking, Nature 239, 500-504]. Bacteria in the presence of an attractant will undergo prolonged running responses with low tumbling frequency [Berg and Brown, 1072; Amsler, C. D. (1996) Use of computer-assisted motion analysis for quantitative measurements of swimming behavior in petrichously flagellated bacteria, Anal. Biochem., 235, 20-25]. To observe the effects of synthetic ligands on tumbling frequency, E. coli were treated with galactose or galactose-bearing ligands, and bacterial motion was recorded and analyzed using the method of Sager et al. [Sager, B. M., Sekelsky, J. J., Matsumura, P., Adler, J. (1988) Use of a computer to assay motility in bacteria, Anal. Biochem., 173, 271-277]. The tumbling frequency was assessed by averaging the mean angular velocity of the paths obtained in the first 5-15 seconds after addition of attractant (FIG. 2A). When bacteria were treated with increasing concentrations of galactose, the mean angular velocity decreased, indicative of a running response. FIGS. 2B-E illustrate sample paths for representative bacteria treated with buffer alone, galactose, compound 1 and compound 3. Treatment with monovalent compound 1 produced similar effects to that of the free chemoattractant (galactose), indicating that the anomeric substituent in 1 did not preclude chemotactic activity. Multivalent compound 3 was more active than monovalent 1 or unmodified galactose. Multivalent compound 3 induced a low mean angular velocity even at very low (e.g., 0.001 mM) saccharide residue concentrations. The response of the bacteria to 3 at 0.1 mM saccharide residue concentrations (ca. 0.004 mM concentration) was comparable to that obtained at ten fold higher (1 mM) concentrations of unmodified galactose. The observed differences in concentration of maximum activity between the monomer 1 and multivalent compound 3 demonstrate that ligand valency affects chemotactic activity.

E. coli were subjected to concentration gradients of compounds 1-4 in capillary accumulation assays [Adler, J. (1973) A method for measuring chemotaxis and use of the method to determine optimum conditions for chemotaxis by Escherichia coli, J. Gen. Microbiol., 74, 77-91] to determine the concentration at which the maximum chemotactic response is achieved and the number of bacteria that accumulate at this maximum [Adler et al., 1973].

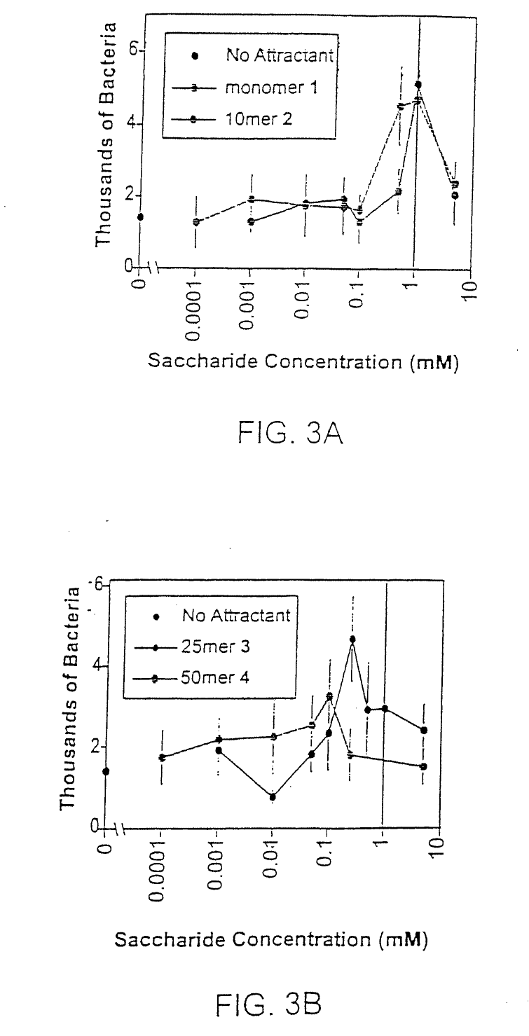

When compounds 1-4 were used as attractants in the capillary accumulation assay, oligomer 2 was no more active than monovalent 1; both elicited a maximum chemotactic response at 1 mM (FIG. 3A). Compound 2 displays a higher local concentration of galactose to the receptor, however, the similarity of activities for 1 and 2 indicates that a high local concentration of attractant does not alone give rise to increased chemotactic activity. For compounds 3 and 4, concentrations of maximum chemotaxis were significantly lower; the maximum for 3 is at a galactose residue concentration of 0.25 mM (ca. 0.01 mM ligand concentration, 100-fold lower than free galactose) and the maximum for 4 is at a galactose residue concentration of 0.10 mM (0.0002 mM ligand concentration). Concentrations of maximum chemotaxis of 3 and 4 are 100- and 5000-fold lower, respectively, than free galactose (FIG. 3B). The ligands of higher valency (3 and 4), therefore, can induce chemotaxis at extremely low concentrations.

Chemotaxis receptors have been found to be approximately 90 Å apart [Barnakov, A. N., Downing, K. H., Hazelbauer, G. L. (1994) Studies of the structural organization of a bacterial chemoreceptor by electron microscopy, J. Struct. Biol. 112, 117-124]. Molecular modeling studies indicate that the maximum length of oligomer 2 is approximately 50 Å [Kanai, M., Morten, K. H., Kiessling, L. L. (1997) Varying the size of multivalent ligands: The dependence of concanavalin A binding on neoglycopolymer length, J. Am. Chem. Soc. 119, 9931-9932]. The significantly higher potency of the longer oligomers 3 and 4, compared to that of oligomer 2, is believed to be due to the ability of the longer oligomers to cluster chemotaxis receptors. Compound 1 was not a chemoattractant for ggbp (AW550 and AW543) or trg (AW701) E. coli mutants. The results obtained indicated that the ligands 1-4 act specifically to affect chemotaxis through the galactose-sensing machinery.

The number of E. coli accumulated in assays employing 1-4 (see FIGS. 3A and 3B) is less than that when galactose is used as an attractant (120,000 bacteria [Adler et al., 1973]), despite the observed potency of these ligands in the video assays (see FIG. 2A). Capillary accumulation assays depend on proper bacterial reorientations to travel into the capillary for collection. The potency of these ligands may disrupt the ability of bacteria to reorient, decreasing the apparent number of bacteria accumulated. At a given saccharide residue concentration of a multivalent ligand, fewer molecules are present to activate the receptors, and these molecules must traverse the outer membrane. These features of the system may also contribute to the decreased numbers of bacteria accumulated.

To test the generality of the observed valency-dependent differences in chemotactic activities and to investigate the role of membrane permeability in responses to our ligands, chemotactic experiments in B. subtilis were conducted. B. subtilis is a gram-positive bacterium that, like gram-negative E. coli, is able to respond to saccharide chemoattractants [Ordal, G. W., Villani, D. P., Rosendahl, M. S. (1979) Chemotaxis towards sugars by Bacillus subtilis, J. Gen. Microbiol., 115, 167-172; Ordal, G. W. (1985) Bacterial chemotaxis: Biochemistry of behavior in a single cell, Crit. Rev. Microbiol., 12, 95-130.]

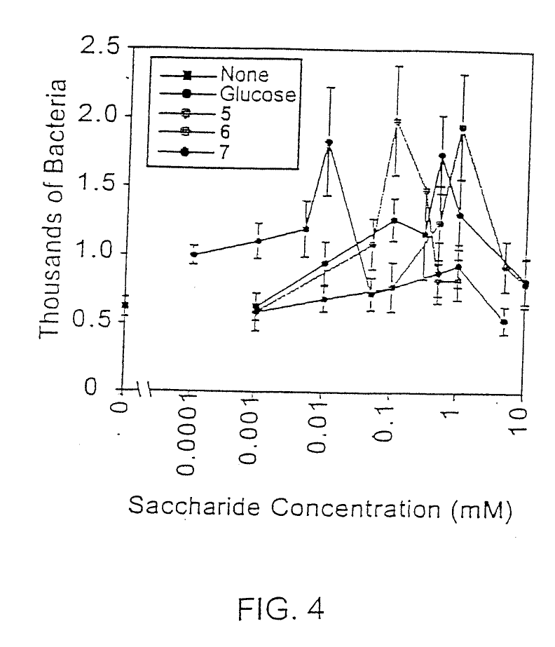

In the case of B. subtilis, the multivalent ligands can directly interact with saccharide-sensing receptors, without having to first traverse the outer membrane. Glucose is a chemoattractant for B. subtilis [Ordal, et al., 1979], but galactose is not. Glucose-carrying ligands (compounds 5-7, Scheme 1) were effective chemoattractants for B. subtilis as shown in capillary accumulation assays. In addition the chemotactic responses to glucose-carrying ligands were shown to also depend on ligand valency. As shown in FIG. 4, monomer 5 elicited maximum activity at 1 mM, while oligomer 6 elicited maximum activity at a saccharide residue concentration of 0.1 mM (50-fold lower ligand concentration than that of free glucose) and oligomer 7 elicited maximum activity at a saccharide residue concentration of 0.01 mM (1250-fold lower ligand concentration than that of glucose). Free chemoattractant signal glucose had maximal activity as a chemoattractant at 0.5 mM. In analogy to observations with E. coli, as the valency of the ligand increases, the saccharide residue concentration of maximum chemotaxis decreases. Significantly, the number of bacteria accumulated towards 5-7 was comparable to the number accumulated when unmodified glucose was used as the attractant. Consistent with previous reports on the activity of galactose, galactose-bearing ligands (such as 1) were not chemoattractants for B. subtilis [Ordal et al., 1979], further indicating that the multivalent ligands were acting specifically. The results observed indicate that in evolutionarily divergent bacteria E. coli and B. subtilis, the valency of the attractant influences chemotactic response.

Fluorescence microscopy experiments were performed to visualize changes in chemotaxis receptor organization upon treatment with saccharide-carrying ligands. These experiments can determine directly whether or not multivalent ligands can influence chemoreceptor reorganization. It had been shown that wild-type E. coli localize chemoreceptors to their poles and that inactivation of the structural protein, CheW, results in a random distribution of chemoreceptors on the cell [Maddock, J. R., Shapiro, L. (1993) Polar location of the chemoreceptor complex in the Escherichia coli cell, Science 259, 1717-1723]. The ability of ROMP-derived arrays to localize the chemoreceptors was examined using E. coli cheW mutants. Bacteria were treated with 1, 3, or 4, fixed, and labeled with an antibody to the bacterial chemoreceptors (anti-Tsr). Monovalent compound 1 had no effect on receptor distribution, but multivalent compounds 3 and 4 were observed to reorganize the chemoreceptors. As anticipated, localized receptors in the cheW cells occurred at seemingly random locations, in contrast to the polar localization observed in the wild type bacteria. Receptor clustering was more pronounced in the case of cells treated with the longer oligomer 4 than with 3. The results of these experiments indicate that ROMP-derived multivalent compounds can induce lateral receptor reorganization. The differences observed in chemotactic activities of the multivalent ligands as a function of oligomer length and the observation that ROMP-derived multivalent ligands can induce lateral receptor reorganization supports the conclusion that receptor reorganization is involved in mediating the chemotaxis response. The results further indicate that changes in receptor localization can give rise to changes in chemotactic responses.

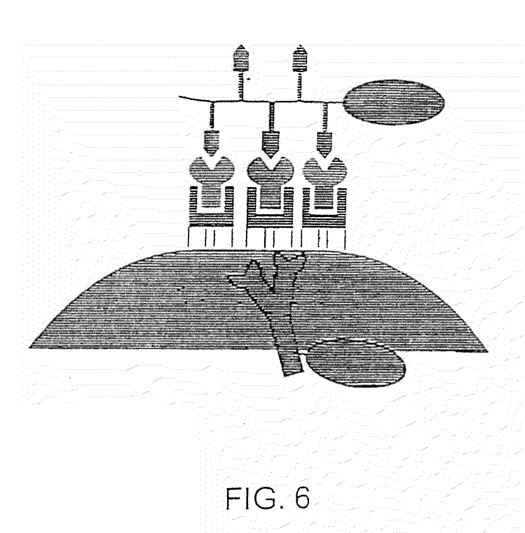

To confirm the ability of multivalent ligands to alter the organization of the chemoreceptors in the bacterial membrane E. coli were treated with compound 8, a galactose-bearing multivalent ligand having a fluorescent label (Scheme 1). When E. coli were treated with 8 or a fluorescein-labeled anti-Tsr antibody 14, the fluorescence patterns observed were similar. Both materials were observed to bind at the poles of the bacteria indicating that the ROMP-derived ligands bind specifically to the bacterial chemoreceptors. To address directly the ability of these multivalent ligands to reorganize receptors 15, CheW mutants were treated with both compounds. Patches of anti-Tsr antibody labeled chemoreceptors that colocalize with compound 8 were observed, as illustrated in FIG. 6. This result indicates that multivalent ligand 8 is responsible for the observed changes in cell receptor organization.

The data obtained indicate that multivalent ligands influence chemotactic responses by altering the organization of cell surface chemoreceptors. An alternative view is that these changes are derived from increases in functional affinity, which result from multivalent presentation. While this mechanism is possible, evidence linking changes in ligand affinities with chemotactic activity is lacking. Equilibrium-binding constants for various ligands often do not correlate with ligand activities in bacterial chemotaxis assays [Adler et al., 1973; Hazelbauer et al., 1971; Boos, W. (1969) The galactose binding protein and its relationship to the -methylgalactoside permease from Escherichia coli. Eur. J. Biochem. 10, 66-73; Yaghmai, R., Hazelbauer, G. L. (1993) Strategies for differential sensory responses mediated through the same transmembrane receptor. EMBO J. 12, 1897-1905].

In contrast, a number of studies have implicated receptor localization in chemotaxis [Jasuj a et al., 1999; Bray et al., 1998; Levit et al., 1998; Duke et al., 1999; Barnakov et al., 1994; Li, G., Weis, R. M. (2000) Covalent modification regulates ligand binding to receptor complexes in the chemosensory system of Escherichia coli, Cell, 100, 357-365]. It has been shown, for example, that assembled tetramers of the chemoreceptor Tar are more active in in vitro signaling than are individual receptors or dimers [Cochran, A. G., Kim, P. S. (1996) Imitation of Escherichia coli aspartate receptor signaling in engineered dimers of the cytoplasmic domain, Science 271, 1113-1116].

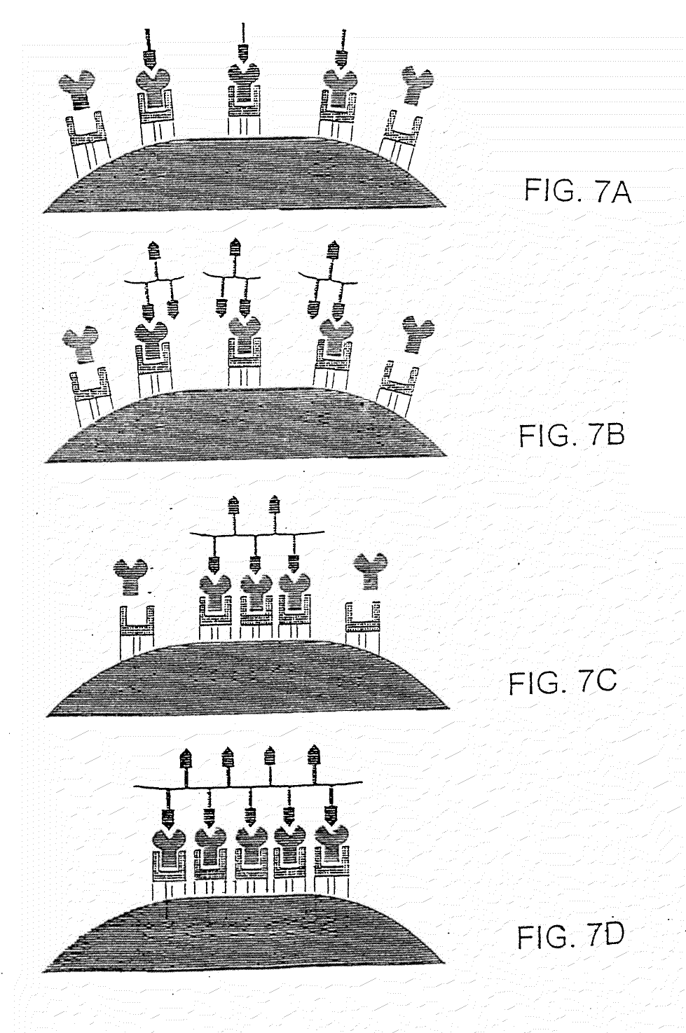

Together, the present data and these results suggest that the differences in chemotactic activities for monovalent 1 versus multivalent 3 and 4 are due to their abilities to control the valency of receptor clusters. Based on these result, a mechanism in which systematic increases in ligand valency lead to changes in chemotactic responses by incorporation of additional receptors into clusters (as illustrated in FIGS. 7A-D) is proposed.

By generating synthetic molecules using ROMP that differ only in ligand valency, as opposed to ligand density or spacing, it has been shown that the valency of a ligand influences its ability to organize chemoreceptors and its ability to elicit a chemotactic response from those receptors. The results demonstrate that multivalent ligands of distinct valency (distinct or defined number of functional moieties), such as those described herein, can be used to tune cellular responses through changes in receptor organization. Further, ligand valency can be used to tune chemotactic responses of diverse bacteria (both E. coli and Bacillus subtilis) indicating that the methods of this invention are generally applicable to diverse cell types. The ROMP-based synthetic route to multivalent arrays is general [Strong, L. E., Kiessling, L. L. (1999) A general synthetic route to defined, biologically active multivalent arrays, J. Am. Chem. Soc. 121, 6193-6196] and can be employed to generate a variety of multivalent ligands or arrays which carry a variety of types and numbers of chemical signals that bind to cell receptors (cell surface receptors, transmembrane receptors and cytoplasmic receptors) and which as a result, likely mediated by lateral receptor reorganization, elicit a biological response. Control of the type of signal covalently bonded to the multivalent ligand and control of the spacing and number of signals presented on the ligand can be used to tune the type and magnitude of the response elicited.

It has also been found that multivalent ligands that bind to one type of receptor can affect the biological response induced by binding of ligands to another type of receptor.

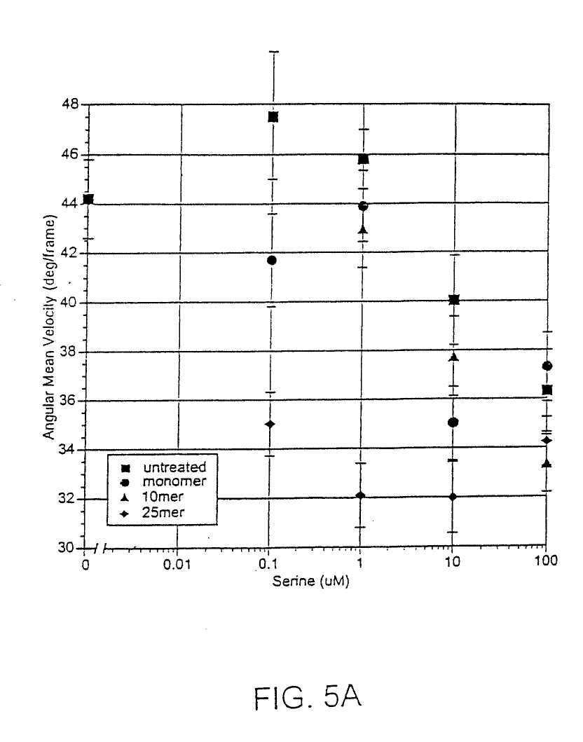

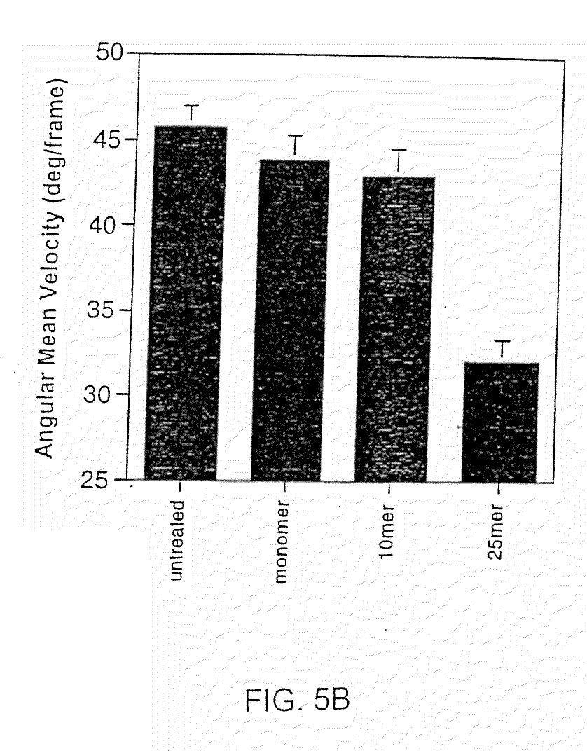

Serine is another small molecule (in addition to galactose) which acts as a chemoattractant for bacteria, such as E. coli. Initial contact of E. coli cells with a multivalent ligand with galactose SREs, compounds 2 and 3 was followed, after a 2 min adaptation period, by addition of varying concentrations of serine. The chemoattractant effect of serine was enhanced about 30%, measured as average mean velocity (deg/frame) (see FIGS. 5A and 5B), in the presence of multivalent ligands compared to serine in the absence of the multivalent ligand. It is believed that clustering of galactose-binding cell receptors by the multivalent ligand caused the enhancement of the response of the cell to the other chemoattractant serine, see FIG. 1.

### Example 2

### Modulation of Neutrophil Chemotaxis

Neutrophil migration is an example of cell migration. Neutrophils migrate toward a number of different endogenous and exogenous substances. N-formyl peptides, bacterial protein degradation products, are one type of exogenous substance that is a chemoattractant for neutrophils [Ye, R., Boulay, F. (1997) Leukocyte Chemoattractant Receptors, Advances in Pharmacology 39:221], a bacterial transcription by product. Neutrophils have cell surface receptors which bind to the chemoattractant and can sense increasing concentration gradients of the chemoattractant. Neutrophils respond to the chemoattractant by migrating toward increased concentrations leading them to the site of infection, for example. In addition, and also in response to such chemoattractants, neutrophils release intercellular signals that affect responses in other cells, particularly other immune systems cells. Multivalent ligands of this invention can be used to enhance the response of neutrophils to chemoattractants and enhance immune system clearance of infectious agents. Scheme 2 illustrates an exemplary N-formyl peptide 20 and an exemplary SRE for that N-formyl peptide 21 for use in multivalent ligands that modulate neutrophil migration. These signal groups (SREs) can be covalently or noncovalently bonded to ROMP scaffolds such as those illustrated in Scheme 2 (22 and 23). Scheme 3 provides exemplary linkers that can be employed in multivalent ligands carrying N-formyl-peptides.

### Example 3

### Modulation of Immune Processes

The development of an immune response can be modulated via valency-dependent interactions of immune system cells with multivalent ligands of this invention. The recognition of foreign (non-self) epitopes, cells, viruses or viral particles for clearance by the immune system is due in part to cell receptors that recognize the epitopes, cells, viruses or viral particle as foreign. In order for clearance to occur, the foreign signal must be recognized and there must be a B cell or T cell response to the foreign signal. Proper immune responses require activation and subsequent deactivation of B cells and T cells. Receptor clustering on B cells and T cells has been implicated in the production of an immune response.

Multivalent ligands of this invention which have one or more BRE or SRE through which the ligand can bind to a B cell, T cell or other immune cell and which carry one or more antigens, epitopes can be employed to modulate the response of the immune cell (enhancing or decreasing immunogenicity of the antigen or epitope). When the epitope or antigen is recognized as foreign (non-self) by the immune cell, cells or immune system in which an immune cell is found, then the multivalent ligand can be used to tolerize the immune cell, cells or immune system to the epitope or antigen. In this case, the epitope or antigen is that of a beneficial or clinical species (cell, particle, nucleic acid) or of a self cell (or tissue) that is incorrectly recognized as foreign (non-self). In contrast, a multivalent ligand of this invention can be used to sensitize or increase the sensitivity of the immune cell, cells or immune system to the foreign epitope or antigen enhance its immunogenicity and enhance the immune response to it. This method would be employed with a foreign epitope or antigen that was not beneficial, e.g., one associated with a pathogen. When the epitope or antigen is recognized as self by the immune cell, cells or immune system in which an immune cell is found, then the multivalent ligand can be used to sensitize the immune cell, cells or immune system to the self epitope or antigen. In this case, the epitope or antigen may be of a non-beneficial self cell or macromolecule, e.g., a cancer cell, or may be a foreign epitope or antigen that is incorrectly recognized as self. In contrast, a multivalent ligand of this invention can be used to tolerize the immune cell, cells or immune system to a self epitope or antigen that is incorrectly recognized as foreign. Methods for tolerization and sensitization are specifically exemplified hereafter.

The C3d complement fragment binds the CR2 receptor (CD21/CD19 complex) on B cells. The expression fusion product of the fusion of the cloned C3d gene fragment and the C-terminal region of hen egg lysozyme gene was able to increase immunogenicity significantly more (1000-fold) than the level achieved with the lysozyme combined with a strong adjuvant [Lou, D., Kohler, H. (1998) Enhanced molecular mimicry of CEA using photoaffinity crosslinked C3d peptide, Nature Biotechnology, 16, 458-462].

Scheme 4 illustrates an exemplary multivalent ligand containing two different signal groups 30 prepared from the ROMP polymer 29 by selective covalent bonding of the different signals. One of the signals is a hen egg lysozyme (HEL) peptide (specific for the A20 cell line):

| | | |
|---|---|---|
| 103-117 | NGMNAWVAWRNRCKG | (SEQ ID NO: 1) |

[Gapin, L., et al. (1998) Antigen presentation by dendritic cells focuses T cell responses against immunodominant peptides: studies in the hen egg-white lysozyme (HEL) model, J. Immunology, 160, 1556-1564] and the other is a 16-mer C3d peptide involved in binding to CR2:

| | |
|---|---|
| KNRWEDPGKQLYNVEA | (SEQ ID NO: 2) |

[Low et al., 1998]. This HEL peptide can be attached to the polymer backbone at the N-terminal amine (40) of the peptide or at a side group of a lysine near the end of the peptide (41):

| | | |
|---|---|---|
| 40: | *GDGNGMNAWVAWRNR-CONH₂ | (SEQ ID NO: 3) |
| or | | |
| 41: | DGNGMNAWVAWRNRGK*-CONH₂ | (SEQ ID NO: 4) |

where * indicates the site of attachment. The C3d peptide can be attached to the multivalent ligand via the thiol of cysteine positioned at either end of the peptide (42 and 43):

| | | |
|---|---|---|
| 42: | *CKNRWEDPGKQLYNVEA | (SEQ ID NO: 5) |
| or | | |
| 43: | KNRWEDPGKQLYNVEAC* | (SEQ ID NO: 6) |

Multivalent ligands containing signals 41 alone or in combination with 42 or 43 or 40 alone or in combination with 42 or 43 can induce an enhanced immune response compared to HEL its self. A multivalent ligand containing a plurality of peptide elements that are ligands for the CR2 receptor can cluster the CR2 receptor on the surface of the B cell and as demonstrated in the chemotaxis experiments can enhance the response of that B cell to other ligands, e.g., antigens. Multivalent ligands containing one or more bound CR2 ligands in combination with one or more bound antigens can cluster the CR2 receptor with the receptor that recognizes the antigen and thereby enhance the response of the B cell to the antigen. Clustering of CR2 with a receptor that recognized HEL (for example, or other antigens) on the B cell surface can enhance the response of the B cell for the HEL antigen and can result in an enhancement of immune response toward the HEL epitope.

An alternative hen egg lysozyme peptide that can be employed in construction of multivalent ligands of this type is:

| | | |
|---|---|---|
| 44: | ELAAAMKRHGLDNYRGYSLGNWVCA. | (SEQ ID NO: 7) |

CD22 is a B cell surface glycoprotein involved in cell adhesion and activation [64]. CD22 is important in the negative regulation of B cell antigen receptor signaling [Blasioli, J. Paust, S., Thomas, M. L. (1999) Definition of the Sites of Interaction between the Protein Tyrosine Phosphatase SHP-1 and CD22, J. Biol. Chem. 274, 2303-2307]. The structure recognized by CD22 is Sia12α6 Galβ14GlcNAcβ (Scheme 5, compound 50). This compound can be attached to a ROMP polymer backbone as illustrated in Scheme 5 via a primary thiol group (compound 51). Multivalent ligands containing one or more ligands for CD22 (such as 51) in combination with one or more HEL epitopes (such as 42 or 43) attenuate the immune response to the HEL epitope.

### Example 4

### Crosslinking (Aggregation) of Cells

Many proteins, such as lectins and antibodies, possess multiple ligand binding sites. When these proteins bind to ligands immobilized on adjacent cell surfaces, the cells aggregate. Cell aggregation can be monitored easily, and this property has found use in the development of diagnostics for pathogen detection [Kemp, B. E., Rylatt, D. B., Bundesen, P. G., et al. (1988) Autologous red cell agglutination assay for HIV-1 antibodies: Simplified test with whole blood, Science 241, 1352-1354], therapeutics [Kawauchi, H., Hosono, M., Takayanagi, Y., Nitta, K. (1993) Agglutinins from aquatic insects-tumor cell agglutination activity. Experientia 49, 358-361; Coloma, M. J., Morrison, S. L. (1997) Design and production of novel tetravalent bispecific antibodies, Nature Biotechnol. 15, 159-163; Segal, D. M., Weiner, G. J., Weiner, L. M. (1999) Bispecific antibodies in cancer therapy, Curr. Opin. Immunol. 11, 558-562], blood typing tests [Furata, M., Uchikawa, M., Ueda, Y., et al. (1998) Construction of mono- and bivalent human single-chain Fv fragments against the D antigen in the Rh blood group: Multimerization effect on cell agglutination and application to blood typing, Protein Eng. 11, 233-241], and other biotechnological applications [Singh, R. S., Tiwary, A. K., Kennedy, J. F. (1999) Lectins: Sources, activities, and applications. Crit. Rev. Biotech. 19, 145-178; Matsuya, Y., Yamane, I. (1985) Cell hybridization and cell agglutination 1. Enhancement of cell hybridization by lectins, J. Cell Sci. 78, 263-271; Takamatsu, H., Kawajiri, H., Takahashi, Y., Manaf Ali, A., Yoshimoto, T. (1999) Continuous antibody production by phytohemagglutinin-L-aggregated hybridoma cells, J. Immunol. Methods 223, 165-170].

Many lectins have been shown to have mitogenic activities that are dependent on the valency of the lectin. These mitogenic lectins, including ConcanavalinA (ConA), are thought to cluster glycoproteins on the surface of the target cell, activating mitogenic signals and inducing cell proliferation [Ramaschi, G., Torti, M., Sinigaglia, F., Balduini, C. (1993) Intracellular Calcium Mobilization is Triggered by Clustering of Membrane Glycoproteins in Concanavalin A-stimulated Platelets, Cell Biochem. Function 11, 241-249; Kaplan, M. R., Trubniykov, E., Berke, G. (1997) Fluorescence Depolarization as an Early Measure of T Lymphocyte Stimulation, J. Immunol. Methods 201, 15-24.] Lectins have been useful tools for exploring signal transduction [Walzel, H., Blach, M., Hirabayashi, J., Kasai, K.-I., Brock, J. (2000) Involvement of CD2 and CD3 in Galectin-1 Induced Signaling in Human Jurkat T-cells. Glycobiology 10, 131-140; Wenzel-Seifert, K., Krautwurst, D., Lentzen, H., Siefert, R. (1996) Concanavalin A and Mistletoe Lectin I Differentially Activate Cation Entry and Exocytosis in Human Neutrophils: Lectins May Activate Multiple Subtypes of Cation Channels. J. Leukocyte Biol. 60, 345-355] and cell growth [Stewart, R., Allan, D. W., McCraig, C. D. (1996) Lectins Implicate Specific Carbohydrate Domains in Electric Field Stimulated Nerve Growth and Guidance, J. Neurobiol., 30, 425-437; Rott, O., Charreire, J., Cash, E. (1996) Influenza A Virus Hemagglutinin is a B-cell-superstimulatory Lectin, Med. Microbiol. Immunol., 184, 185-193], and studies using them have elucidated possible functional roles for mammalian lectins, such as the galectins and selectins.

The effectiveness of multivalent proteins at instigating cell aggregation is determined by how tightly the protein binds to cell surface ligands. One effective way to increase the avidity of these interactions is to increase the number of ligand binding sites [Crothers, D. M., Metzger, H. (1972) The influence of polyvalency on the binding properties of antibodies, Immunochem. 9, 341-357; Mammen, M., Choi, S.-K., Whitesides, G. M. (1998) Polyvalent interactions in biological systems: implications for design and use of multivalent ligands and inhibitors, Angew. Chem. Int. Ed. Engl. 37, 2755-2794; Kiessling, L. L. & Pohl, N. L. (1996) Strength in numbers: Non-natural polyvalent carbohydrate derivatives. Chem. Biol. 3, 71-77]. Research efforts have focused on favoring oligomer formation for lectins [Osawa, T., Beppu, M. (1987) Cross-linked derivatives of concanavalin A. Methods Enzymol. 150, 17-28; Hudson, P. J., Kortt, A. A. (1999) High avidity scFv multimers; diabodies and triabodies, J. Immunol. Methods 231, 177-189] or generating novel multimers of antibody scFv fragments [Hudson, P. J., Kortt, A. A. (1999) High avidity scFv multimers; diabodies and triabodies, J. Immunol. Methods 231, 177-189]. Methods which further enhance the number of binding sites or favor the optimized orientation of these binding sites would increase the utility of these materials in many applications.

Lectins are a large class of saccharide-binding proteins, many of which are homo-oligomers assembled from two to four copies of identical subunits [Weis, W. I., Taylor, M. E., Drickamer, K. (1998) The C-type lectin superfamily in the immune system, Immunol. Rev. 163, 19-34]. Lectins aggregate cells when they crosslink glycoproteins or glycolipids on adjacent cell surfaces. Aggregation can be modulated by altering the number of active monomers within the lectin oligomer. For example, the ability of the tetravalent mannose-binding plant lectin concanavalin A (ConA) to aggregate red blood cells is greatly decreased when the lectin is forced into a lower valency dimeric form by succinylation [Osawa et al. 1987]. Increasing the valency of lectins may have the opposite effect, i.e. to enhance cell aggregation; however, methods have not been readily available for generating lectin complexes with higher order valencies. Because the valency of ROMP-derived materials can be altered systematically, the effect that the number of saccharide groups, such as mannose, bound to the ligand has on the number of lectins, such as ConA, assembled on a given scaffold can be investigated.

The precipitation of ConA depends on the clustering of ConA tetramers and this technique can be used to determine the stoichiometry of insoluble ConA—ligand complexes [Khan, M. I., Mandal, D. K., Brewer, C. F. (1991) Interactions of concanavalin A with glycoproteins. A quantitative precipitation study of concanavalin A with the soybean agglutinin, Carbohydr. Res. 213, 69-77]. To investigate the formation of ConA clusters with multivalent ligands of this invention, ROMP-based scaffolds containing defined numbers of mannose residues, the monomer 9 and polymers 10-13 having n of 10, 25, 50, or 100, respectively, illustrated in Scheme 1 were prepared using ROMP methods [Strong & Kiessling, 1999]. Compounds 9-13 were contacted with ConA, monomeric compound 9 was unable to induce precipitation, but multivalent compounds 10-13, caused concentration-dependent precipitation of ConA. Precipitation results further indicated that the stoichiometry of ConA complexed with 10 (the 10-mer) is about 2:1 and that of complexes of ConA with 11 and 12 is approximately 4:1.

In contrast, dimeric succinylated ConA precipitated only with the highest valency multivalent ligand compounds tested, e.g., compound 12, and the complexes formed had a 4:1 (receptor:scaffold) stoichiometry in the precipitate. Thus, the number of mannose residues displayed by the scaffold is important in the formation of protein-scaffold complexes. Precipitation results were substantially confirmed with a transmission electron microscopy (TEM) technique in which clusters of biotinylated ConA with compounds 10-12 were labeled with a high density streptavidin—gold particle. Compound 10 was observed to form dimers exclusively, while 11 was able to form both dimers and trimers and compound 12 formed both dimers and trimers as well, but favored trimeric clusters more than the other scaffolds.

The assembly of ConA clusters in solution can be monitored by fluorescence resonance energy transfer (FRET), in which fluorescein and tetramethylrhodamine (TMR) serve as donor and acceptor fluorophores [Ballerstadt, R., Schultz, J. S. (1997) Competitive-binding assay method based on fluorescence quenching of ligand held in close proximity by a multivalent receptor, Anal. Chim. Acta, 345, 203-212; Burke, S. D., Zhao, Q., Schuster, M. C., Kiessling, L. L. (2000). Synergistic formation of soluble lectin clusters by a templated multivalent saccharide ligand. J. Am. Chem. Soc., 122, 4518-4519]. When these fluorophores are within approximately 80 Å the fluorescein signal is quenched, such that fluorescein fluorescence should decrease when labeled ConA is assembled into clusters. Compounds 9-12 were added to a solution of fluorescein- and TMR-labeled ConA. The fluorescence emission maximum of fluorescein was monitored to ascertain which scaffolds promoted the formation of ConA clusters. In agreement with the previous experiments, ConA clusters formed in the presence of multivalent ligands 10-12 but not with monomeric compound 9. The fluorescence quenching was dependent not only on scaffold valency, but also on ligand concentration. Quenching first increased as scaffold concentration increased and then decreased again as the concentration was increased further. The absence of quenching at high scaffold (multivalent ligand) concentrations indicates that ConA clusters are disfavored at these concentrations, likely because of site saturation. The high concentration of scaffold compared to ConA favors occupation of each ligand binding site on ConA by individual polymers precluding clustering of multiple lectins.

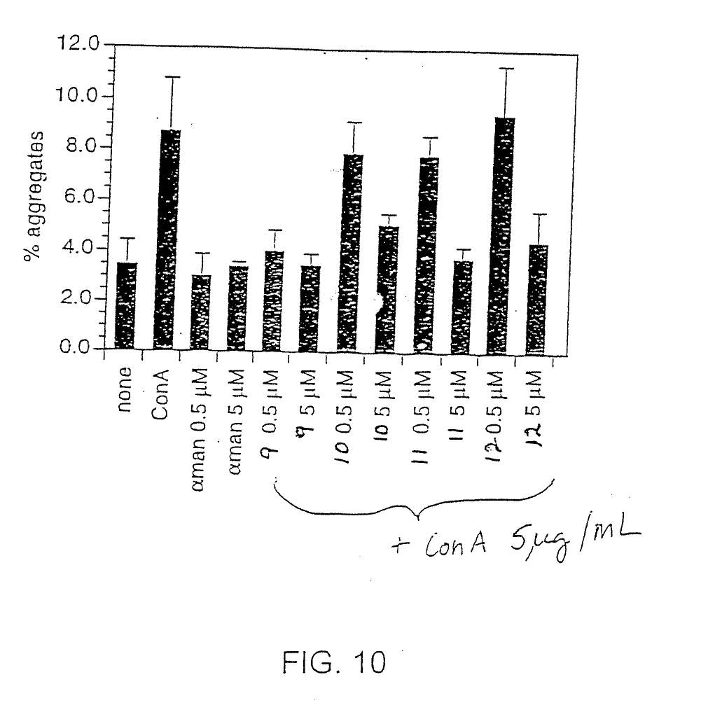

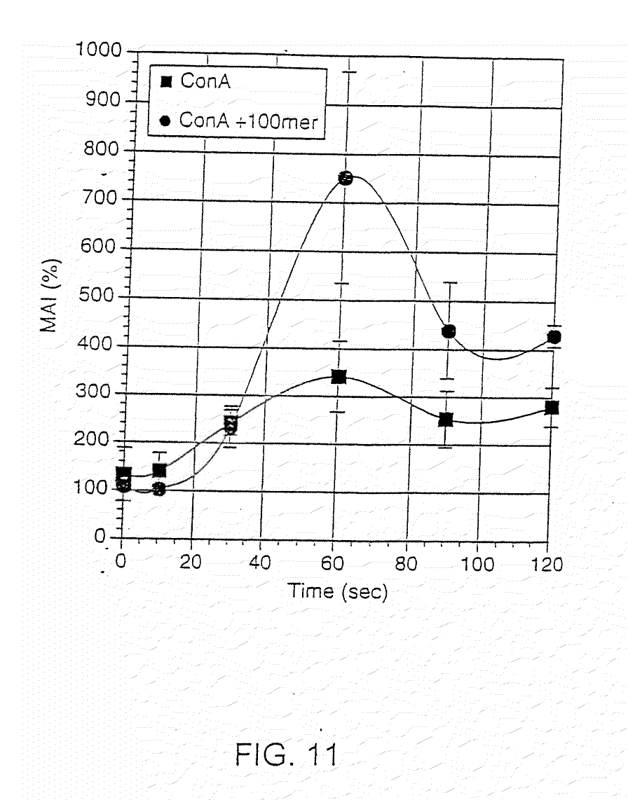

The ability of ConA clusters formed on ROMP-derived polymers to aggregate Jurkat cells was examined initially by light microscopy (see FIG. 10). ConA alone was able to induce some Jurkat cell aggregation even at low concentrations (5 μg/mL). When monovalent ConA ligands such as methyl α-D-mannopyranoside or 9 were premixed with ConA they inhibited aggregation, presumably by destabilizing ConA—cell interactions. For Jurkat cells, inhibition occurred even at low concentrations (0.5 μM) of monovalent ligands. Interestingly, multivalent compounds 10-12 did not inhibit Jurkat cell aggregation at 0.5 μM, a concentration shown to be optimum for ConA cluster formation under similar conditions. Increasing the concentration of the multivalent ligand 10-fold (5 μM) abolished aggregation activity, consistent with site saturation. Thus it is possible to alternatively inhibit or promote cell surface-lectin interactions by varying scaffold valency and multivalent ligand concentration. The ability of ConA complexed to multivalent ligands to interact with cell surfaces was thus tunable.

Further experiments were conducted which demonstrated that ConA-mediated agglutination of erythrocytes could be controlled by addition of multivalent ligands (compounds 9-13). Certain combinations of ConA and multivalent ligands exhibited enhanced agglutination of these cells compared to ConA itself, as shown in FIG. 11. In particular, a combination of ConA tetramer and multivalent ligand (compound 13) at concentration ratio 10:1 (based on tetrameric ConA and based on the number of mannose residues) exhibited significantly enhanced agglutination compared to ConA alone.

Complexes containing multiple ConA tetramers were assembled readily on compounds 10-13 when intermediate multivalent ligand concentrations were used, but were not detectable when the concentration of the scaffold was either too low or too high. The concentration range over which such complexes are formed depends upon the relative concentrations of ConA and multivalent ligand (based on the number of ligands, RE or SRE) and upon the valency of multivalent ligand. This is generally true for any complex of a multivalent ligand with any protein. The concentration range over which complexes of a multivalent ligand with one or more ConA (or such complexes with any lectin or more generally with any protein) can be readily determined for a particular application under particular conditions by assessing retention of function by ConA (or more generally the protein or lectin). Complexes of multivalent ligands with ConA will generally be formed, dependent upon the valency of the multivalent ligand and the particular experimental conditions, when the concentration range of the ligand (based on numbers of SRE, e.g., mannose) ranges from about 1:1 to over 100:1.

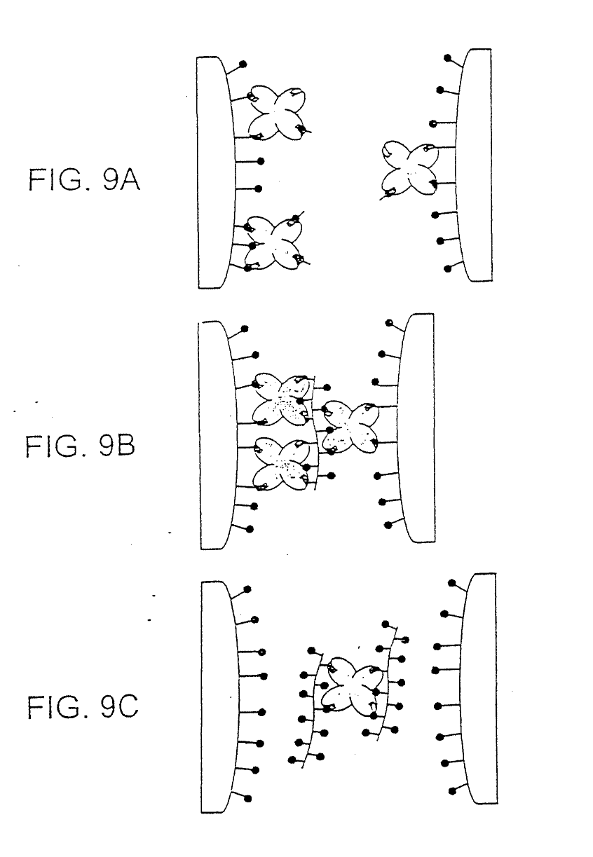

The results herein indicate generally that the valency and concentration of a multivalent ligand can be varied to control the assembly of lectin on to the multivalent scaffolds of these multivalent ligands. More specifically, the valency and concentration of ROMP-derived materials can be varied to control the formation of ConA clusters, as illustrated in FIGS. 9A-9C. Monovalent ligands (as well as low concentrations of multivalent ligands) ligands bind to lectin, but do not inhibit cell aggregation (FIG. 9A). Under conditions that favor lectin-scaffold complexation, i.e., intermediate concentration levels of multivalent ligands, a plurality of lectins can be assembled on the multivalent ligand and the lectins retain free saccharide binding sites capable of interacting with cell surfaces (FIG. 9B). When multivalent ligand concentration is increased, lectin binding sites are saturated by binding to a plurality of multivalent ligands, lectin assembly is disfavored and lectins are not capable of interacting with cell surfaces (FIG. 9C). Thus, as illustrated, scaffold valency and ligand concentration can be controlled to assemble lectin clusters with multivalent ligands wherein the lectin retains cell binding activity. Further, scaffold valency and more importantly multivalent ligand concentration can be controlled to inhibit the cell aggregation function of lectins.

These results demonstrate that proteins, such as lectins, can be assembled on a polymeric scaffold, such as those provided by the multivalent ligands of this invention, and that the assembled proteins, including lectins, will retain biological function. Methods described herein can be employed to generate polymeric assemblies of one or more lectins, as well as polymeric assemblies of one or more antibodies or antibodies fragments, which retain the ability to bind to ligands (e.g., saccharides or epitopes). Methods herein are generally applicable to generation of assemblies of various chemical and biological species, particularly macromolecular species, including proteins, carbohydrates, nucleic acids though binding to recognition elements and signal recognition elements in a multivalent ligand.

Enhancement of Cell Toxicity Using Multivalent Ligands: Lectins, such as ConA, as well as agglutinins and phytohemaglutinins in general, can exhibit toxic effects in certain kinds of cells. Multivalent ligands carrying saccharide groups can complex with lectins, such as ConA, as discussed above. Complexes containing several lectin molecules complexed to an appropriately substituted multivalent ligand can function to aggregate cells, if binding sites on the lectin are not saturated by binding to the ligand groups. When higher multivalent ligand concentrations (dependent upon the specific conditions and applications, and dependent upon ligand valency) are employed, lectin binding sites can become saturated and cell aggregation by the lectin is then inhibited. Saturation of a given lectin by a given multivalent ligand can be readily determined empirically. Further, saturation of the function of any protein by a given multivalent ligand can be determined by assessing function of the complexed protein.

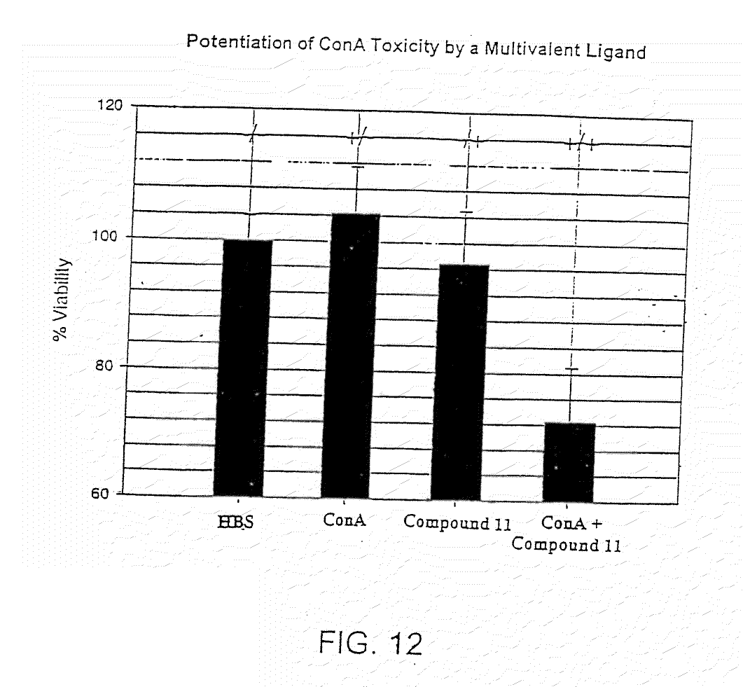

Complexes of a lectin with multivalent ligands have been found to exhibit cell toxicity that is enhanced over that of the lectin itself. As illustrated in FIG. 12, PC12 cells treated with 0.1 μM ConA (for 48 hr) exhibited no apparent loss of viability. In contrast, PC12 cells treated with combination of 0.1 μM ConA and 4 μM of compound 11 under the same conditions exhibit almost a 30% loss in viability. These results indicate that complexes of lectin with multivalent ligands of this invention in which the ratio of the concentrations of ligand to lectin is sufficiently high to saturate ligand binding sites of the lectin can trigger apoptosis in cells.

### Example 5

### Materials and Methods

Generation of Multivalent Polymers: ROMP was used to convert 1 to the series of oligomers 2-4 as previously described [Manning, D. D., Strong, L. E., Hu, X., Beck, P. J., Kiessling, L. L. (1997) Neoglycopolymer inhibitors of the selectins, Tetrahedron 53, 11937-11952]. Similar conditions were employed in the synthesis of oligomers 6 and 7 [Strong & Kiessling, 1999]. Fluorescent polymer 8 was generated by specific end-labeling with a bifunctional capping agent [Scheme 7] and subsequent conjugation to the fluorophore BODIPY-TR (commercially available from Molecular Probes) [Gordon, E. J., Gestwicki, J. E., Strong, L. E., Kiessling, L. L. (2000) A bifunctional capping agent for ROMP: synthesis and cell binding of a fluorescent neoglycopolymer. Chem. Biol. 7, 9-16]. Compounds 9-12 were the samples prepared and tested in reference [Strong & Kiessling, 1999]. The degree of polymerization (dp) for each compound was determined by ¹H NMR. Valency (n) is an approximation of the degree of polymerization (DP), the ratio of monomer to catalyst used in ROMP.

Video Microscopy: E. coli AW405, which exhibits wild-type chemotactic responses, from an overnight culture were grown in LB (Luria Bertani broth) to OD₅₅₀ of 0.4-0.6 and then washed twice with attractant-free chemotaxis buffer (10 mM potassium phosphate buffer, pH 7.0, 10 ΦDM EDTA). Partially permeabilized bacteria (25 μM EDTA for 3 min. at room temperature, then quench with 50 μM CaCl₂) at an OD₅₅₀ of 0.1 were placed under a cover slip supported by additional cover slips in the method of Sager et al., 1988. (Permeabilization had no effect on bacterial chemotaxis toward galactose or 1 but was necessary for chemotaxis toward 4 [Leive, L., Kollin, V. (1967) Controlling EDTA treatment to produce permeable Escherichia coli with normal metabolic processes, Biochem. Biophys. Res. Comm. 28, 229-236]). Bacteria were allowed to adjust to contact with glass surface for 1-2 min. Attractant was added to achieve the final concentration indicated at a 5 ΦL final volume. The bacterial motion at 28 EC was recorded, and the paths were analyzed using the ExpertVision system. Paths derived from the first 5 to 15 seconds following the introduction of attractant were analyzed. Angular mean velocities varied approximately 14% between experiments performed on different days. Data were analyzed using the Q and Students tests.

Capillary Accumulation Assay: E. coli from an overnight culture were grown in LB to OD₅₅₀ 0.4-0.6, washed twice with E. coli chemotaxis buffer, and then partially permeabilized. Bacteria were resuspended in chemotaxis buffer to an OD₅₅₀ 0.1 and utilized in the capillary accumulation assay at 30° C. for 60 mM, as previously described [Adler, 1973]. B. subtilis 011085 was grown from an overnight culture in T broth (1% tryptone, 0.2 mM MgCl₂, 0.5% NaCl, 0.01 mM MnCl₂) supplemented with 10 mM glucose and 0.5% glycerol, washed with B. subtilis chemotaxis buffer (10 mM phosphate buffer, pH 7.0, 10 ΦM EDTA, 0.5% glycerol, 0.3 mM (NH₄)₂SO₄), and capillary assays were performed at a final OD₅₅₀ 0.01 at 37 EC for 30 min [Ordal et al., 1979]. The number of B. subtilis accumulated was normalized to 500 bacteria accumulated towards buffer alone. Results of capillary assays can be influenced by factors other than the activity of the attractant, such as metabolism of the substrate or toxicity [Adler, 1973; Adler, J. (1966) Chemotaxis in bacteria, Science, 153, 708-716]. To exclude this possibility, we tested the ability of E. coli to utilize 1 as a sole carbon source. These experiments revealed that 1-4 are not toxic and that monomer 1 is not metabolized (data not shown). Data was analyzed using the Q and Students tests.

Immunofluorescence Microscopy: E. coli AW405 or RP1078 (cheW) were pretreated with buffer alone or with compounds 1, 3, 4, or 8 at 5 mM in a 10 ΦL total volume of chemotaxis buffer. After a 10 minute incubation at 30° C., the bacteria were fixed (2% paraformaldehyde (PFA) in HEPES pH 7.0, 30 min., 4° C.), placed on poly-L-lysine treated cover slips in the bottom of 6-well plates, permeabilized with methanol, and labeled with anti-Tsr antibody (1:250) and fluorescein-labeled goat-anti-rabbit antibody (1:500) according to the procedure of Maddock and Shapiro [Maddock, J. R., Shapiro, L. (1993) Polar location of the chemoreceptor complex in the Escherichia coli cell, Science, 259, 1717-1723]. Anti-Tsr antibodies recognize the conserved chemoreceptor cytoplasmic domain and are thus cross-reactive with multiple chemoreceptors. Some binding exclusion (exclusive 530 nm or 590 nm fluorescence at a pole) was seen in dual labeling experiments in which both antibody and 8 were used. Fluorescence microscopy was performed on a Zeiss Axioscope at 1000× using an oil immersion lens. Images were captured using IPLab Spectra 3.2 and Adobe PhotoShop 5.0.

Quantitative Precipitation: Quantitative precipitations and analysis were carried out by a method modified from that previously described by Khan et al., 1991. Briefly, ConA (Vector Laboratories, Burlingame, Calif.) and scaffold were dissolved in precipitation buffer (0.1 M Tris-HCl pH 7.5, 90 μM NaCl, 1 mM CaCl₂, 1 mM MnCl₂), vortexed briefly to mix, and then incubated for 5 hours at room temperature (or 2 days at 4° C. for succinylated ConA). The final concentration of ConA tetramers was 30 μM (assuming ConA tetramers with a molecular weight of 104,000) and succinylated ConA dimers was 44 μM (assuming dimers with a mass of 52,000). White precipitates were pelleted by centrifugation at 5000×g for 2 minutes. Supernatants were removed by pipet and pellets were gently washed twice with cold buffer. Pellets were then resuspended in 600 μL 100 mM methyl α-D-mannopyranoside (100 μL for succinylated ConA), and were completely dissolved after a 10 minute incubation at room temperature. Protein content was determined by measuring the absorbance at 280 nm by UV-vis spectroscopy on a Varian Cary 50 Bio using a 100 μL volume quartz cuvette. Measurements are the average of three independent experiments.

Transmission Electron Microscopy: TEM methods were preformed essentially as previously described [Gestwicki, J. E., Strong, L. E. Kiessling, L. L. (2000) Visualization of single multivalent receptor-ligand complexes by transmission electron microscopy, Angew. Chem. Int. Ed. 39, No. 24, 4567-4570]. ConA tetramers were labeled with biotin using conditions that favored attachment of 1-2 copies of biotin residues. Biotinylated ConA tetramers were mixed with ligands of interest in solution and then contacted with an excess of streptavidin-conjugated 10 nm gold particles. Samples can be treated with 2% phosphotungstic acid (pH 7.0, 30 sec) to enhance contrast. Images of random fields were acquired for each treatment and analyzed for formation of ConA complexes. Gold particles within 25 nm of less of each other were considered to be part of a complex. This distance was based on the modeled length of the synthetic multivalent ligands used [Kanai et al., 1997] and the structure of tetrameric ConA determined by X-ray crystallographic analysis [Parkin, S., Rupp, B., Hope, H. (1996) Atomic resolution structure of concanavalin A, Acta Crystallogr. Sect. D 52:1161-1168].

Specifically, biotinylated ConA (2.3 μM) and scaffold (0.75 μM, mannose concentration) in PBS pH 7.2 were incubated for 15 minutes at room temperature before streptavidin—10 nm gold (Sigma, St. Louis, Mo.) was added to a final concentration of 3.0 μM. Complexes were incubated at room temperature for 15 minutes and then placed onto carbon-coated Formvar-treated grids. Grids were air dried and viewed on a LEO Omega 912 Energy Filtering Electron Microscope (EFTEM). Images were at 12,500× magnification, collected on a ProScan Slow Scan CCD camera, and analyzed in Adobe PhotoShop 5.0. Fields averaged between 5 and 50 gold particles and 15-20 fields were collected on each day for each treatment. Results are the average of results obtained on three separate experiments performed on independent samples on three separate days. Total number of gold particles collected on each day varied from about 80 to over 400.

Fluorescence Resonance Energy Transfer: Fluorescein-ConA (Vector Laboratories, Burlingame, Calif.) and TMR-ConA (Sigma, St. Louis, Mo.) in phosphate buffered saline (PBS) pH 7.2 were mixed to afford final concentrations of 4 μg/mL and 0.4 μg/mL respectively. Scaffold was added in PBS to the final concentrations indicated, with a final volume of 200 μL. This solution was vortexed briefly and then incubated at room temperature for at least 15 minutes. No precipitates were observed in any of these samples. Fluorescence was determined on a Hitachi F-4500 fluorospectrophotometer using a 200 μL volume quartz cuvette, an excitation wavelength of 492 nm, emission wavelength of 515 nm, and 10 nm slit widths. Emission intensities are the average of 3-5 independent experiments with 3 scans performed during each experiment. Compounds 9-12 had negligible fluorescence at 515 nm. Curves were fit using the equation:

% F=(% Fₘₐₓ×L)/(L+IC₅₀)

where % F is the change in fluorescence relative to untreated, % Fₘₐₓ is the maximal recovery of fluorescence, L is the micromolar mannose concentration of scaffold, and IC₅₀ is the half-maximal concentration for inhibition of clustering.

Jurkat Cell Aggregation: Jurkat cells were cultured and maintained as previously described [Gordon et al., 2000]. Cells were washed three times in cold PBS and then treated with Hoescht 33342 (100 μg/mL) for 30 minutes at 30° C. Cells were washed twice with cold PBS and then fixed for 30 minutes at 4° C. with 2% paraformaldehyde in HEPES pH 7.4. Fixed cells were washed twice and then treated in 200 μL final volume with premixed solutions of ConA and scaffold. A 2× solution of ConA and scaffold was prepared in PBS pH 7.2, vortexed briefly, and then incubated at 22° C. for 30 minutes before being added to cells. Cells, ConA solutions, and 100 μg/mL DNAse (to prevent cell aggregation by nucleic acid) were incubated at 22° C. for 30 minutes. Cells were pelleted at 400×g, resuspended gently into 50 μL PBS, and then added to slides for visualization at 200× magnification on a Zeiss Axioscope outfitted with the appropriate filter set. Approximately 100-200 cells were counted from random fields on each day. Clusters were scored for at least two cells in direct contact with each other and expressed as a percentage of the total number of objects (individual cells and clusters) counted. Results are summarized in FIG. 10. ROMP-derived ligands 9-12 alone were not able to cause cell aggregation. Images were captured in IPlab Spectra 3.2 and prepared in Adobe Photoshop 5.0.

Erythrocyte Agglutination: ConA (53 nM, 5 μg/mL) and ligand compound 13 (530 nM; per saccharide basis) were added in an end final volume of 100 μL, PBS ph 7.2 in a 96-well plate. The complexes were incubated for 15 minutes at room temperature.

Cell Toxicity Experiments: HBS buffer contained HEPES (10 mM), NaCl (150 mM), and CaCl₂ (1 mM) at pH=7.4. Concanavalin A (ConA) was obtained from Vector Labs (Burlington, Calif.) and was freshly diluted for all experiments. The concentration of the ConA stock solution was determined using A₂₈₀^{1%}=13.7 (95). A single ConA dilution was then made and split for each sample. Ligands were then added from appropriate stock solutions at 5 times the desired final concentration. All samples had six replicates for each concentration. Control samples were used in each run that contained HBS alone, ConA in HBS and the highest concentration of ligand in HBS. Lysis controls were made by adding HBS buffer alone and adding lysis buffer after 48 h sample incubation.

Cell Culture: All cell culture reagents were obtained from GIBCO BRL unless otherwise noted. PC12 cells (ATCC: CRL-1721) were grown in media containing 84% (v/v) RPMI 1640 (with L-glutamine), 5% (v/v) heat inactivated fetal bovine serum, 10% (v/v) heat inactivated horse serum, and 1% penicillin/streptomycin (10000 units/ml), in a humidified incubator at 37 C and 5% CO₂. Low serum media contained 97.5% (v/v) RPMI 1640 (with L-glutamine), 0.5% inactivated fetal bovine serum, 1% (v/v) heat inactivated horse serum, and 1% penicillin/streptomycin (10000 units/ml). Cells were grown in T-flasks treated with collagen, and harvested at confluence by trypsinization (0.05% trypsin and 0.4 mM EDTA) followed by quenching with fresh medium. Cells were concentrated to pellet (2100 rpm for 10 min), aspirated then resuspended in fresh medium. The population was determined by haemocytometer and treatment with trypan blue, cells were then plated to 96-well plates (tissue culture treated obtained from CoStar, Corning N.Y.) at ˜15,000 cells/well. Plates were then incubated for 34 h to allow cells to adhere.

The medium was then removed and replaced with low serum medium (80 μL). Samples and controls in HBS (20 μL) were then added and incubated for 48 h at 37 C. After incubation 10 μL of a 5 mg/mL solution of MTT (3-(4,5-dimethylthiazol-2-yl)-2,5-diphenyltetrazolium bromide (Sigma/Aldrich, Milwaukee, Wis.) in low serum RPMI medium was added to each well.

After 4 h, 100 μL of lysis buffer (50% dimethyl formamide/20% sodium dodecyl sulfate in HBS, pH=4.7) was added and the cells were incubated overnight. The plate was then read on a plate reader (Biostar) at 570 nm. Percent cell viability was determined using the following equation:

$$\frac{{G}_{1} - {G}_{0}}{{G}_{con} - {G}_{0}} = \% V ,$$

where G₀ is the lysed cell control, G_{con} is control cells treated only with vehicle, and G₁ is a sample treated with peptide and vehicle. Results from an experiment in which cells are initially treated with multivalent polymer compound 11 followed by treatment with ConA and appropriate controls are illustrated in FIG. 11.

### Example 6

### A Nickel-Chelating Polymer Scaffold for Protein Oligomerization

Very few well-defined biochemical tools exist that allow the study of higher order protein clusters, which are ubiquitous to biology. Important examples of systems thought to require multivalent protein-protein interactions are FGF-mediated cell signaling and the immune adherence phenomenon. [D. M. Ornitz (2000) FGFs, heparan sulfate and FGFRs: complex interactions essential for development, Bioessays, 22, 108-112; G. Waksman and A. B. Herr (1998) New insights into heparin-induced FGF oligomerization, Nat. Struct. Biol., 5, 527-530; J. Taipale and J. KeskiOja (1997) Growth factors in the extracellular matrix Faseb J., 11, 51-59; J. A. Schifferli and R. P. Taylor (1989) Physiological and Pathological Aspects of Circulating Immune-Complexes, Kidney Int., 35, 993-1003; J. E. Gestwicki, L. E. Strong, C. W. Cairo, F. J. Boehm and L. L. Kiessling (2002) Cell aggregation by scaffolded receptor clusters, Chem. Biol., 9, 163-169; C. W. Cairo, J. E. Gestwicki, M. Kanai and L. L. Kiessling (2002) Control of multivalent interactions by binding epitope density, J. Am. Chem. Soc., 124, 1615-1619.]

This example describes the design, synthesis, and biological activity of a well-defined polymer scaffold for the non-covalent oligomerization of proteins. The reported scaffold takes advantage of the well characterized nickel-His-tagged protein interaction and therefore represents a general scaffold for the oligomerization of any His-tagged protein. Griffith B. R., et al. (2004) J. Amer. Chem. Soc. 126(6): 1608-9 provides details of a nickel-chelating scaffold and this reference is incorporated by reference herein for details of synthesis and analysis.

The utility of binding proteins to immobilized nickel through a poly-histidine tag has been exploited in protein purification, western blot detection, immobilization of proteins onto surfaces for SPR studies, and immobilization of proteins onto microtiter plates for ELISA. [E. Hochuli, W. Bannwarth, H. Dobeli, R. Gentz and D. Stuber (1988) Genetic Approach to Facilitate Purification of Recombinant Proteins with a Novel Metal Chelate Adsorbent, Bio-Technology, 6, 1321-1325; R. Gentz, C. H. Chen and C. A. Rosen (1989) Bioassay for Trans-Activation Using Purified Human Immunodeficiency Virus Tat-Encoded Protein—Trans-Activation Requires Messenger-Rna Synthesis, Proc. Natl. Acad. Sci. U.S.A., 86, 821-824; D. J. Oshannessy, K. C. Odonnell, J. Martin and M. Brighamburke (1995) Detection and Quantitation of Hexa-Histidine-Tagged Recombinant Proteins on Western Blots and by a Surface-Plasmon Resonance Biosensor Technique Anal. Biochem., 229, 119-124; P. D. Gershon and S. Khilko (1995) Stable Chelating Linkage for Reversible Immobilization of Oligohistidine Tagged Proteins in the Biacore Surface-Plasmon Resonance Detector, J. Immunol. Methods, 183, 65-76; G. B. Sigal, C. Bamdad, A. Barberis, J. Strominger and G. M. Whiteside (1996) A self-assembled monolayer for the binding and study of histidine tagged proteins by surface plasmon resonance, Anal. Chem. 1996, 68, 490-497; L. R. Paborsky, K. E. Dunn, C. S. Gibbs and J. P. Dougherty (1996) A nickel chelate microtiter plate assay for six histidine-containing proteins Anal. Biochem., 234, 60-65.

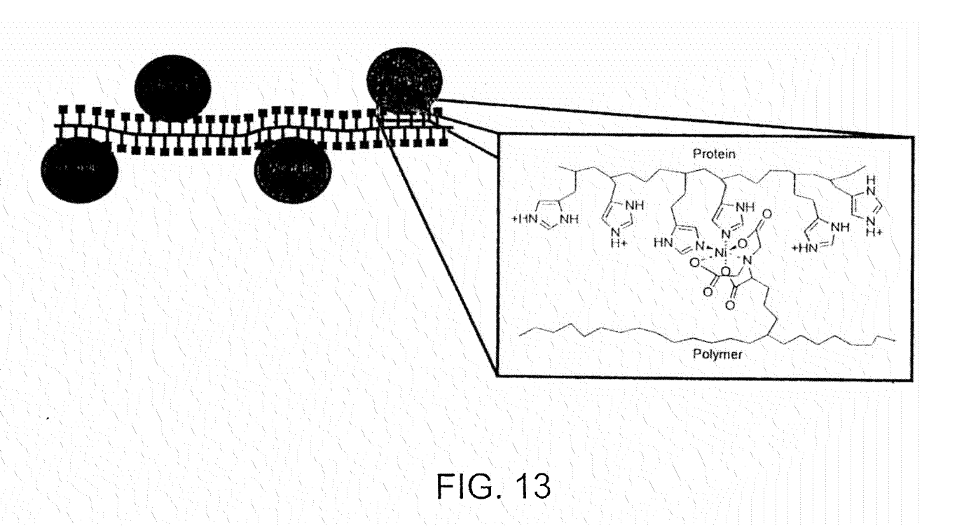

The interaction between a single chelated nickel atom and a single His-tagged peptide/protein is fairly weak (Kd˜3 μM). [D. J. Oshannessy, K. C. Odonnell, J. Martin and M. Brighamburke (1995) Detection and Quantitation of Hexa-Histidine-Tagged Recombinant Proteins on Western Blots and by a Surface-Plasmon Resonance Biosensor Technique, Anal. Biochem., 229, 119-124; I. T. Dorn, K. R. Neumaier and R. Tampe (1998) Molecular recognition of histidine-tagged molecules by metal-chelating lipids monitored by fluorescence energy transfer and correlation spectroscopy, J. Am. Chem. Soc., 120, 2753-2763.] Generally, nickel is immobilized and presented to the protein in a multivalent fashion, thereby increasing the binding strength through avidity. This interaction was exploited in a new way by displaying immobilized nickel on a polymeric scaffold (FIG. 13). The nickel-chelating polymer provided exploits His-tags expressed on recombinant proteins and facilitates the oligomerization of those proteins.

An exemplary polymer scaffold was designed to possess two important characteristics—a well defined length and amenability to the determination of relative coupling efficiencies of a nucleophile. The recently published succinimide ester-substituted polymer generated by atom-transfer radical polymerization (ATRP) provided a functionalizable scaffold of well defined length (100). [A. Godwin, M. Hartenstein, A. H. E. Muller and S. Brocchini (2001) Narrow molecular weight distribution precursors for polymer-drug conjugates, Angew. Chem.-Int. Edit, 40, 594-597.]

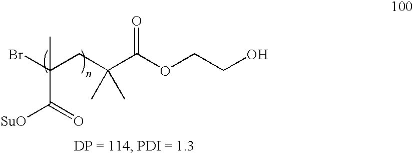

It also provided a terminal primary hydroxyl for orthogonal functionalization. End-labeling of this hydroxyl with TBSCl provided a resolved ¹H NMR signal. See also PCT published application WO01/18080 for descriptions of uniform molecular weight polymers useful for synthesis of Ni-chelator scaffolds of this invention.

Nitriloacetic acid (NTA) was used as the nickel-chelating functionality. Benzyl protecting groups on the NTA derivative (200) provided a second resolved NMR signal for the determination of coupling efficiencies. Additionally, a diglycine linker was added to the NTA-containing nucleophile to provide better accessibility to the polymer backbone at high coupling densities than the more typically used lysine derivative.

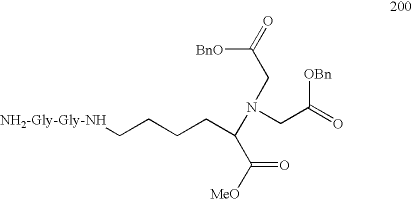

Synthesis of the polymer began with end-labeling of the polymer precursor with TBSCl. The protected NTA derivative was then conjugated under typical succinimide-ester promoted conjugation conditions, followed by quenching unreacted succinimide ester sites with excess ethanolamine (Scheme 8). The functionalized polymer was then deprotected under standard hydrolytic conditions. Ni²⁺ was added under basic conditions, and extensive dialysis yielded the final product.

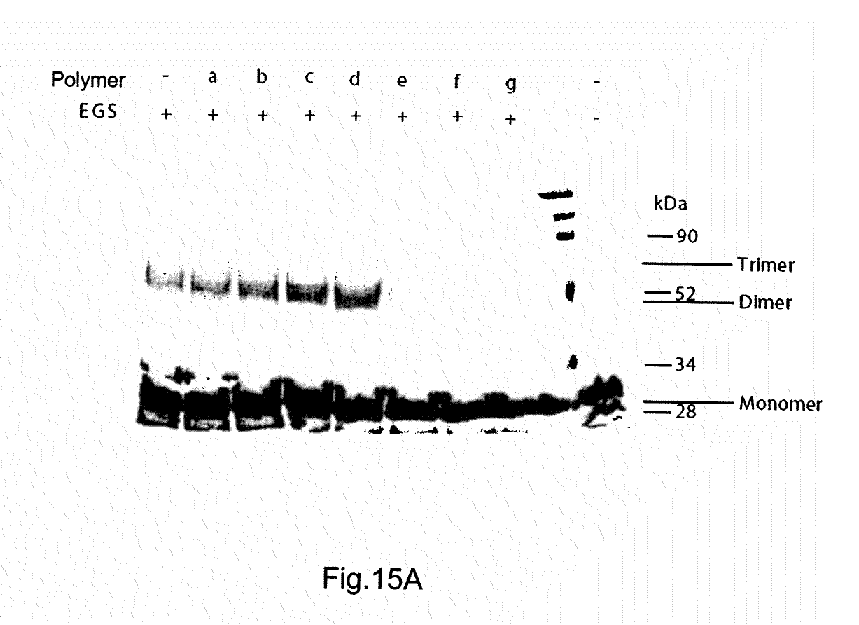

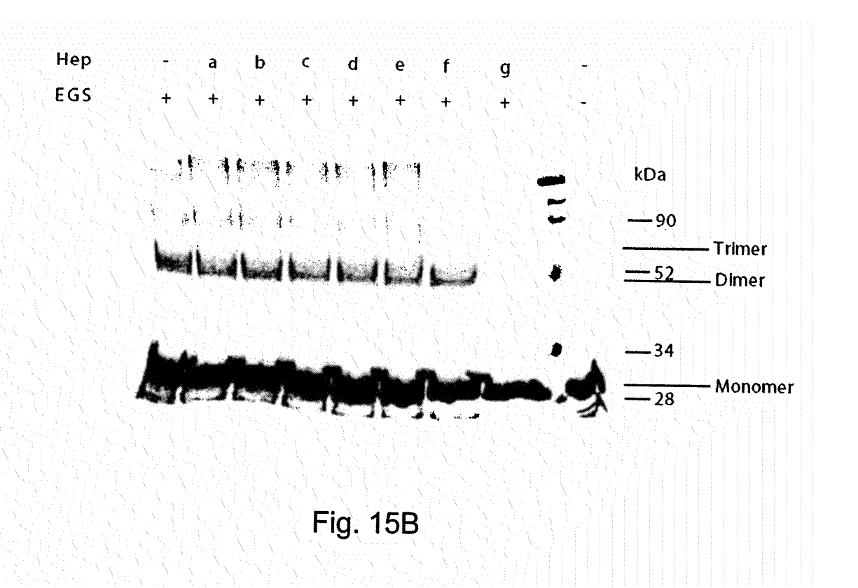

As mentioned previously, FGF-mediated cell signaling has been shown to involve multivalent protein interactions. Specifically, one of the functions of the oligosaccharide heparin in vitro is to oligomerize FGF ligand proteins and present them to the FGF receptor in a multivalent fashion. [T. Spivakkroizman, M. A. Lemmon, I. Dikic, J. E. Ladbury, D. Pinchasi, J. Huang, M. Jaye, G. Crumley, J. Schlessinger and I. Lax (1994) Heparin-Induced Oligomerization of Fgf Molecules Is Responsible for Fgf Receptor Dimerization, Activation, and Cell-Proliferation Cell, 79, 1015-1024; C. P. Kwan, G. Venkataraman, Z. Shriver, R. Raman, D. F. Liu, Y. W. Qi, L. Varticovski and R. Sasisekharan (2001) Probing fibroblast growth factor dimerization and role of heparin-like glycosaminoglycans in modulating dimerization and signaling J. Biol. Chem., 276, 23421-23429; M. W. Pantoliano, R. A. Horlick, B. A. Springer, D. E. Vandyk, T. Tobery, D. R. Wetmore, J. D. Lear, A. T. Nahapetian, J. D. Bradley and W. P. Sisk (1994) Multivalent Ligand-Receptor Binding Interactions in the Fibroblast Growth-Factor System Produce a Cooperative Growth-Factor and Heparin Mechanism for Receptor Dimerization, Biochemistry, 33, 10229-10248; F. J. Moy, M. Safran, A. P. Seddon, D. Kitchen, P. Bohlen, D. Aviezer, A. Yayon and R. Powers (1997) Properly oriented heparin-decasaccharide-induced dimers are the biologically active form of basic fibroblast growth factor, Biochemistry, 36, 4782-4791; A. B. Herr, D. M. Ornitz, R. Sasisekharan, G. Venkataraman and G. Waksman (1997) Heparin-induced self-association of fibroblast growth factor-alpha—Evidence for two oligomerization processes, J. Biol. Chem., 272, 16382-16389.] The polymers synthesized were tested for the ability to mimic this specific oligomerization function of heparin. Preliminary cross-linking studies were performed using a His-tagged FGF ligand protein, FGF-8b, to verify the ability of this polymer to oligomerize FGF-8b. Covalent cross-linking of FGF-8b mediated by the nickel-chelating polymer (FIG. 15A) or heparin (FIG. 15B) was examined.

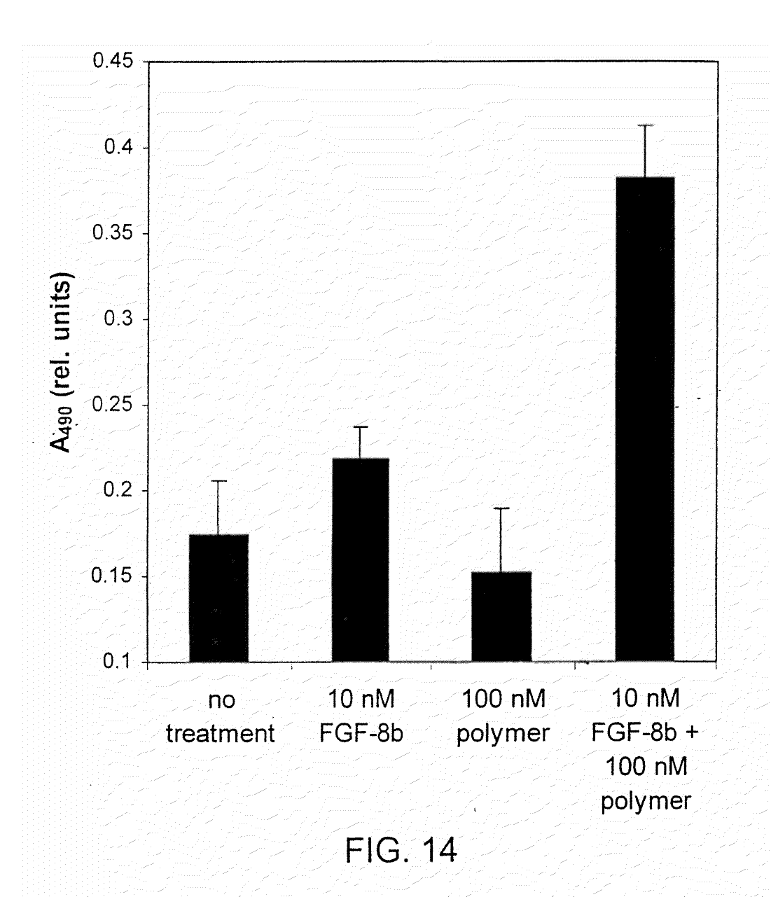

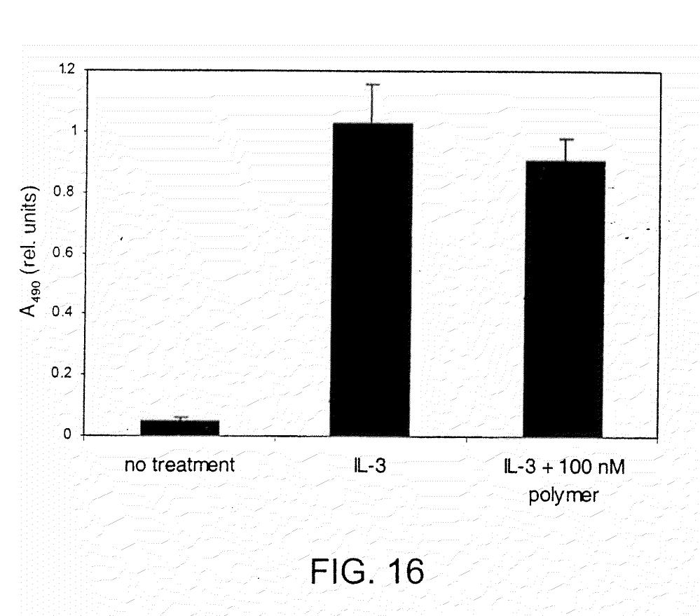

The ability of the polymer to promote FGF-8b-mediated cell proliferation in vitro was tested. Heparan sulfate deficient BaF3 cells transfected with FGF receptor Mc require interleukin-3 (IL-3) containing media to avoid apoptosis and proliferate. Alternatively, these cells can be rescued from apoptosis by incubation with soluble heparin and FGF-8b, but undergo apoptosis when incubated with only heparin or only FGF-8b. The ability of the polymer to promote FGF-8b-mediated proliferation in the absence of heparin/heparan sulfate was tested. The results shown in FIG. 14 demonstrate that the polymer can rescue BaF3 cells from apoptosis. Furthermore, the polymer alone did not demonstrate any nonspecific effect and was not toxic at the highest concentrations used (FIG. 16).

The multivalent ligands of this example provide for the non-covalent oligomerization of His-tagged proteins. This polymeric scaffold can cluster FGF-8b and induce FGF-mediated cell proliferation in the absence of heparin/heparan sulfate. The methods of this example can be employed to prepare aggregates of various other proteins carrying His-tags.

Materials and Methods: BaF3 Proliferation Assay (See FIG. 14)—BaF3 cells transfected with FGF receptor IIIc were counted in a hemacytometer, washed with IL-3 free media, (RPMI+10% calf serum+L-glutamine) and resuspended in the above media at a concentration of 1×10⁵ cells/mL. 100 μL aliquots of cells were added to a flat-bottomed 96-well plate. 1 μL of 1 μM FGF-8b (His-tagged) was added to each well to give a final concentration of 10 nM. 1 μL of 10 μM polymer 6 or heparin (Sigma, porcine intestinal mucosa, 17-19 kDa) was added to the appropriate wells to give a final polymer or heparin concentration of 100 nM. The plates were gently shaken and incubated in a CO₂ incubator at 37° C. for 48-72 hours. 20 μL of MTS reagent (CellTiter 96 Aqueous One, Promega) was added to each well and the plate was incubated as before for 1 hour. The absorbance was measured at 490 nm using a standard ELISA plate reader.

Comparison of cross-linking of FGF-8b mediated by nickel-chelating polymer 206 or heparin (FIGS. 15A and B, respectively): FGF-8b was incubated with polymer 206 for 1 hour at 0° C. The ratio of FGF-8b to polymer was as follows: a. 1:0.001, b. 1:0.003, c. 1:0.01, d. 1:0.03, e. 1:0.1, f. 1:0.3, f. 1:1 (FIG. 15A). EGS (ethylene glycol bis[succinimidylsuccinate]) was added, and cross-linking was allowed to proceed for 5 minutes at 0° C. The reactions were quenched with ethanolamine (excess) for 1 hour at 0° C. Samples were concentrated under vacuum, analyzed on a 12% SDS-PAGE gel under reducing conditions, and detected with Coomassie staining.

FGF-8b was incubated with heparin (Sigma, porcine intestinal mucosa, 17-19 kDa) for 1 hour at 0° C. The ratio of FGF-8b to heparin was as follows: a. 1:0.000001, b. 1:0.00001, c. 1:0.0001, d. 1:0.001, e. 1:0.01, f. 1:0.1, g. 1:1 (FIG. 15B). EGS (ethylene glycol bis[succinimidylsuccinate]) was added, and cross-linking was allowed to proceed for 5 minutes at 0° C. The reactions were quenched with ethanolamine (excess) for 1 hour at 0° C. Samples were concentrated under vacuum, analyzed on a 12% SDS-PAGE gel under reducing conditions, and detected with Coomassie staining.

Both gels show a pattern in cross-linking indicative of oligomerization due to a specific ligand/scaffold interaction. The amount of cross-linking increases with increasing polymer or heparin concentration until an optimal concentration is reached. At concentrations higher than the optimal concentration, site saturation prevents cross-linking.

2-(Bis-benzyloxycarbonylmethyl-amino)-6-tert-butoxycarbonylamino-hexanoic acid methyl ester (207) (See Scheme 10): N^{ε}-tert-butoxycarbonyl-L-lysine, methyl ester hydrochloride (5 g, 17 mmol) was dissolved in 100 mL amine-free DMF at 0° C. The solution became cloudy upon addition of TEA (14 mL, 102 mmol). Benzyl 2-bromoacetate (27 mL, 170 mmol) was added followed by KI (2.8 g, 17 mmol) as a finely crushed powder. The temperature was raised to 60° C., at which time the solution became clear, except for undissolved KI. After stirring for 45 hours the dark brown mixture was subjected to high vacuum evaporation (water bath ≦60° C.) for several hours. The resulting dark brown oily mixture was extracted with 150 mL EtOAc and 200 mL brine. The aqueous layer was re-extracted with 50 mL EtOAc. The combined organic layers were washed with 2×100 mL 10% citric acid, 100 mL saturated NaHCO₃, 100 mL brine, dried over Na₂SO₄, filtered, and the solvent was removed under vacuum to yield a dark brown oil. The oil was purified by silica gel column chromatography using EtOAc/Hexanes (25%-100% EtOAc gradient) to yield a golden brown oil. Yield: 4.8 g (8.6 mmol) of 207, 51%. TLC: R_{f}⁼0.21 in 25% ethyl acetate/hexanes. ¹H NMR (300 MHz, CDCl₃): δ=1.2-1.8 ppm (m, 6H), δ=1.4 ppm (s, 9H), δ=3.1 ppm (q, 2H), δ=3.4 ppm (t, 1H), δ=3.6 ppm (s, 3H), δ=3.7 ppm (s, 4H), δ=5.2 ppm (s, 4H), δ=7.4 ppm (s, 10H). ¹³C NMR (75 MHz, CDCl₃): δ=23.0, 28.3 (3 C), 29.4, 30.0, 40.2, 51.3, 52.6 (2 C), 64.6, 66.3 (2 C), 78.8, 128.2 (6 C), 128.4 (4 C), 135.6 (2 C), 155.9, 171.1 (2 C), 172.9 ppm. MS (ESI): M+⁺H=557.2, calc.=556.2.

5-(Bis-benzyloxycarbonylmethyl-amino)-5-methoxycarbonyl-pentyl-ammonium chloride (208) (See Scheme 10): To 207 (4.2 g, 7.6 mmol) was added 10 mL HCl (4 M in dioxane) at room temperature with vigorous stirring and vigorous evolution of CO₂ was observed. After 1 hour, volatiles were removed under high vacuum to yield a golden brown oily glass. The oily glass was purified on a plug silica gel using MeOH/DCM (5%-20% gradient) to yield a golden brown glass. Yield: 3.2 g (6.5 mmol) of 8, 86%. TLC: R_{f}=0.10 in 5% MeOH/DCM. ¹H NMR (300 MHz, CD₃OD): δ=1.3-1.8 ppm (m, 6H), δ=2.8 ppm (t, 2H), δ=3.5 ppm (t, 1H), δ=3.6 ppm (s, 3H), δ=3.7 ppm (s, 4H), δ=5.1 ppm (s, 4H), δ=7.3 ppm (s, 10H). ¹³C NMR (75 MHz, CD₃OD): δ=23.7, 27.9, 30.5, 40.6, 52.0, 53.9 (2 C), 65.5, 67.4 (2 C), 129.2 (6 C), 129.6 (4 C), 137.4 (2 C), 172.9 (2 C), 174.4 ppm. MS (ESI): M+⁺H=457.1. calc.=456.1.

2-(Bis-benzyloxycarbonylmethyl-amino)-6-{2-[2-(2,2-dimethylpropoxycarbonylamino)-acetylamino]-acetylamino}-hexanoic acid methyl ester (209). (See Scheme 11): BOC-Gly-Gly-OH (1.8 g, 7.8 mmol) was dissolved in 8 mL amine-free DMF and TEA (1.8 mL, 13 mmol) and cooled with stirring to 0° C. HOBT (1.2 g, 7.8 mmol) was dissolved in the solution. HBTU (3.0 g, 7.8 mmol) was added and required warming to room temperature to completely dissolve. The reaction was again cooled to 0° C. and 8 (3.2 g, 6.5 mmol) was added dissolved in 4.5 mL amine-free DMF. The reaction was stirred at 0° C. for 1 hour, then allowed to warm to room temperature overnight and became yellow and cloudy. The cloudy solution was extracted with 100 mL EtOAc and 200 mL brine. The aqueous layer was re-extracted with 2×50 mL EtOAc. The combined organic layers were washed with 2×50 mL 10% citric acid, 50 mL saturated NaHCO₃, 2×50 mL brine, dried over Na₂SO₄, filtered, and the solvent was removed under vacuum to yield a golden brown oil. The oil was purified by silica gel column chromatography using MeOH/DCM (2.5%-20% gradient) to yield a pale yellow oily glass. Yield: 3.7 g (5.5 mmol) of 209, 85%. TLC: R_{f}=0.25 in 5% MeOH/DCM. ¹H NMR (300 MHz, CDCl₃): δ=1.3-1.5 ppm (m, 4H), δ=1.4 ppm (s, 9H), δ=1.6-1.7 ppm (m, 2H), δ=3.1 ppm (t, 2H), δ=3.4 ppm (t, 1H), δ=3.6 ppm (s, 3H), δ=3.65 ppm (s, 4H), δ=3.7 ppm (s, 2H), δ=3.8 ppm (s, 2H), δ=5.1 ppm (s, 4H), δ=7.3 ppm (s, 10H). ¹³C NMR (75 MHz, CD₃OD): δ=24.1, 28.7 (3 C), 29.8, 30.9, 40.2, 43.4, 48.1, 51.9, 53.8 (2 C), 65.9, 67.4 (2 C), 80.9, 129.2 (6 C), 129.6 (4 C), 137.4 (2 C), 158.7, 171.4, 172.9 (2 C), 173.0, 174.6 ppm. MS (ESI): M+⁺H=671.2. calc.=670.2.

({[5-(Bis-benzyloxycarbonylmethyl-amino)-5-methoxycarbonylpentylcarbamoyl]-methyl}-carbamoyl)-methyl-ammonium chloride (2) (See Scheme 11): To 209 (0.94 g, 1.4 mmol) was added 6 mL HCl (4 M in dioxane) and the solution was stirred at room temperature. After 1 hour, the volatiles were removed under high vacuum to yield a pale yellow/green oil which solidified as the temperature decreased under vacuum. The solid was purified on a plug a silica gel using MeOH/DCM (10%-20% gradient) to yield a pale yellow/green oil with some insolubles. Yield: 0.75 g (1.2 mmol) of 2, 88%. TLC: R_{f}⁼0.15 in 10% MeOH/DCM. ¹H NMR (300 MHz, CD₃OD): δ=1.2-1.5 ppm (m, 4H), δ=1.6 ppm (m, 2H), δ=3.1 ppm (t, 2H), δ=3.35 ppm (s, 2H), δ=3.45 ppm (t, 1H), δ=3.6 ppm (s, 3H), δ=3.7 ppm (s, 4H), δ=3.9 ppm (s, 2H), δ=5.1 ppm (s, 4H), δ=7.3 ppm (m, 10H). ¹³C NMR (75 MHz, CD₃OD): δ=24.1, 30.0, 30.9, 40.2, 42.7, 43.3, 51.9, 53.8 (2 C), 65.9, 67.4 (2 C), 129.2 (6 C), 129.6 (4 C), 137.4 (2 C), 170.4, 173.0 (3 C), 174.6 ppm. MS (ESI): M+⁺H=571.1. calc.=570.1.

TBS end-labeled polymer (203) (See Scheme 9): To precursor polymer 100 (330 mg, 0.015 mmol) was added 3.1 mL dry, amine-free DMF and the mixture was stirred at room temperature to dissolve the polymer. In a separate flask TBSCl (330 mg, 2.2 mmol) was dissolved in 0.6 mL dry, amine-free DMF and DIEA (0.5 mL, 2.7 mmol). The TBSCl solution was added dropwise to the polymer solution at room temperature, with immediate production of a white precipitate. After 30 minutes, the solution was precipitated dropwise addition to 150 mL vigorously stirred acetone. The precipitate was allowed to settle to the bottom of the flask (approximately 5 minutes) to facilitate rapid filtration. The precipitate was filtered on a 30 mL medium porosity glass frit. The material was redissolved in 15 mL dry, amine-free DMF and reprecipitated as before. After filtration, the material was stored under high vacuum overnight to yield an off-white solid. Yield: 0.81 g (0.037 mmol) of 3, 82%. ¹H NMR (300 MHz, DMSO-d₆): δ=0.0 ppm (s), δ=0.8 ppm (s), δ=0.9-1.9 ppm (broad), δ=1.9-3.4 ppm (broad).

Protected NTA-bearing polymer (204) (See Scheme 8): In a small glass vial 203 (10 mg, 0.056 mmol) was dissolved in 0.21 mL dry DMSO at 50° C. In a 0.6 mL Ependorf tube 200 (154 mg, 0.25 mmol) was dissolved in 0.15 mL dry DMSO. Approximately ⅙ of the 200 solution (40 μL, 0.042 mmol) was added to the polymer solution. To the reaction vial was then added TEA (15 μL, 0.11 mmol) and DIC (13 μL, 0.084 mmol) and the reaction was stirred at 50° C. After 16 hours the solution was pale yellow. Ethanolamine (13 μL, 0.22 mmol) was added and the reaction was maintained at 50° C. After an additional 4 hours the reaction solution was transferred to a 50 mL solvent-resistant plastic centrifuge tube (pre-rinsed with DMSO/MeOH/acetone) with HPLC-grade MeOH. The material was precipitated by dropwise addition of 10 mL acetone:Et₂O, (1:1) followed by vigorous shaking to yield a white solid. The solid was isolated by centrifugation for 1 minute, redissolved in 2 mL HPLC-grade MeOH (took several minutes to redissolve, which is indicative of a polymer), and re-precipitated with 10 mL acetone:Et₂O (1:1) plus an additional 5 mL Et₂O. The solid was isolated by centrifugation, re-dissolved in HPLC-grade MeOH, and transferred to a small flask. The solvent was removed under vacuum and the flask was stored under high vacuum dessication overnight to yield a glass. Yield: 18 mg (0.036 mmol), 204, 64%. ¹H NMR (300 MHz, CD₃OD): δ=0.1 ppm (s), δ=0.9 ppm (s), δ=0.9-2.8 ppm (broad), δ=2.7 ppm (s), δ=3.0-4.0 ppm (broad), δ=5.1 ppm (s), δ=7.3 ppm (s).

Deprotected NTA-bearing polymer (205) (See Scheme 8): LiOH (110 mg, 2.6 mmol) was dissolved in 3 mL MQ H₂O and 2 mL THF was added to yield 5 mL of a 60:40 solution. The LiOH solution was added to 204 (44 mg, 0.086 mmol, 0.26 mmol ester functionality) at room temperature and the initially insoluble polymer mixture was stirred. The polymer dissolved within 1 hour. After 18 hours, the solution was neutralized with 6 M HCl, and THF was removed under vacuum to yield a clear solution. The material was dialyzed against MQ H₂O in 2,000 MWCO dialysis tubing at 4° C. for 48 hours, with H₂O changes every 12 hours. The solution was lyophilized to dryness to yield a white fluffy solid. Yield: 11 mg (0.030 mmol) 205, 35%. ¹H NMR (300 MHz, CD₃OD): δ=0.7-2.7 ppm (broad), δ=3.1-4.1 ppm (broad).

Nickel-chelating polymer (206) (See Scheme 8): NiCl₂.6H₂O (0.36 g, 1.5 mmol) was dissolved in 1 mL MQ H₂O and added to 205 (10 mg, 0.027 mmol). The pH was adjusted to pH˜9-10 with NH₄OH, and the solution was stirred for 1 hour. The solution was dialyzed against 0.1 M NH₄OAc, pH=7.0 at 4° C. for 48 hours, with buffer changes every 12 hours, then dialyzed against MQ H₂O in the same manner. The material was lyophilized to dryness to yield a white fluffy solid which made a blue/green solution when dissolved in H₂O. The presence of nickel was confirmed by visible spectroscopy. Yield: 10 mg (0.025 mmol) 206, 91%.

### Example 7

### Bifunctional Polymeric Scaffolds For Pathogen Clearance

This example describes new bifunctional conjugates for the clearance of pathogens from the bloodstream. Conjugates are polymeric scaffolds presenting two Fab′ fragments each with a different specificity: one having specificity for a receptor on erythrocytes and the other having specificity for a selected pathogen (fungal, protozoan, bacterial, viral pathogen). More specifically, conjugates present Fab′ fragments for the CR1 receptor present on erythrocytes, and Fab′ fragments for a selected pathogen. In this example the pathogen exemplified is Staphylococcus aureus.

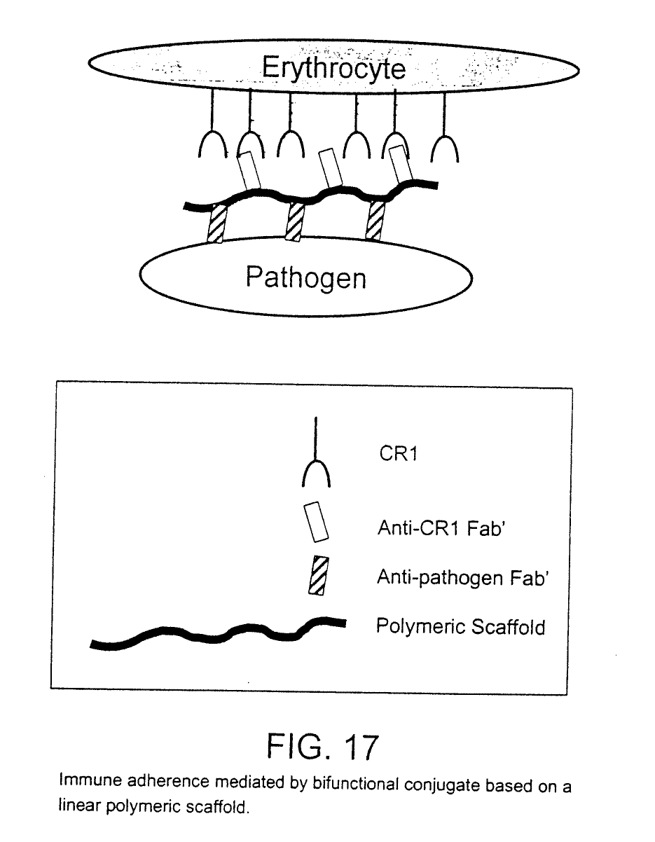

The Fab′ fragments have been conjugated chemoselectively to a maleimide-containing, water-soluble methyl acrylate polymer backbone (FIG. 17). The conjugates of this invention have several advantages over existing cross-linked bispecific antibodies for pathogen clearance. The advantages include a more homogeneous structure, the ability to vary the structure in a defined manner, the ability to ensure an accessible antigen binding site, and a higher binding site to molecular weight ratio. These features of the conjugates of this invention provide for more efficacious and less toxic therapeutics for clearing pathogens from the bloodstream. Additionally, these conjugates are useful as research tools for probing the mechanism of pathogen recognition and destruction in the liver and spleen.

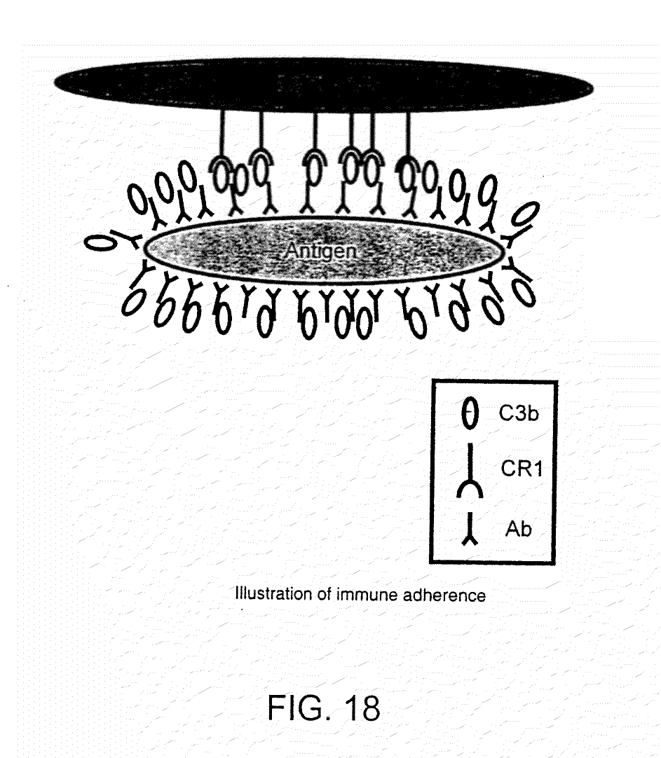

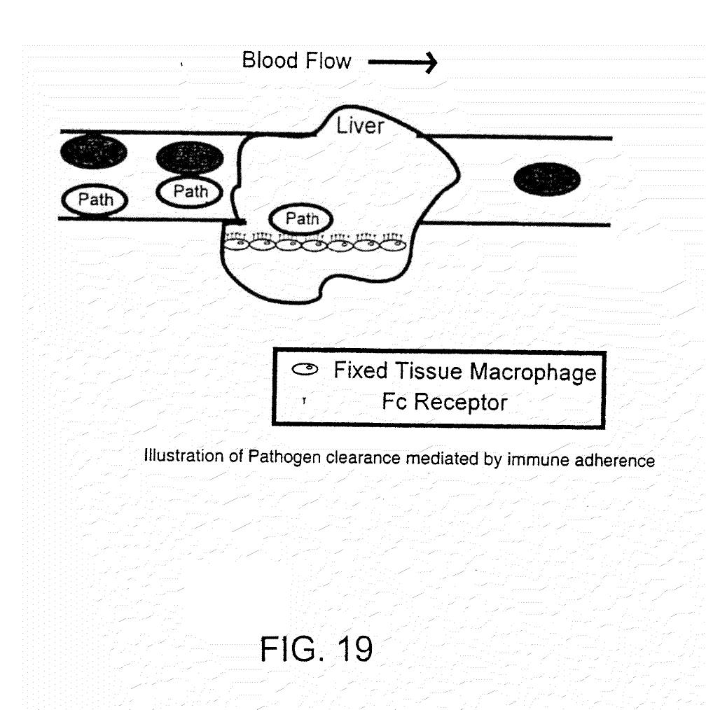

The biology underpinning this strategy is known as the immune adherence phenomenon, first demonstrated by Nelson in 1953 (Nelson, R. A. Science 1953, 118, 733-737. The Immune-adherence phenomenon). Immune adherence involves binding of complement-opsonized antibody/particulate antigen immune complexes (IC) to human erythrocytes through the CR1 receptor (FIG. 18) (Fearon, D. T. Journal of Experimental Medicine 1980, 152, 20-30. Identification of the membrane glycoprotein that is the C3b receptor of the human erythrocyte, polymorphonuclear leukocyte, B lymphocyte, and monocyte.). Circulating immune complexes eventually pass through the liver or spleen, where they are dissociated from the erythrocytes and destroyed, while the erythrocyte recirculates (FIG. 19). (Cornacoff, J. B., Hebert, L. A., Smead, W. L., Vanaman, M. E., Birmingham, D. J. and Waxman, F. J. (1983) Journal of Clinical Investigation, 71, 236-247. Primate Erythrocyte-Immune Complex-Clearing Mechanism; Waxman, F. J., Hebert, L. A., Cornacoff, J. B., Vanaman, M. E., Smead, W. L., Kraut, E. H., Birmingham, D. J. and Taguiam, J. M. (1984) Journal of Clinical Investigation, 74, 1329-1340. Complement Depletion Accelerates the Clearance of Immune-Complexes from the Circulation of Primate; Waxman, F. J., Hebert, L. A., Cosio, F. G., Smead, W. L., Vanaman, M. E., Taguiam, J. M. and Birmingham, D. J. (1986) Journal of Clinical Investigation, 77, 82-89. Differential Binding of Immunoglobulin-a and Immunoglobulin-G1 Immune-Complexes to Primate Erythrocytes In vivo—Immunoglobulin-a Immune-Complexes Bind Less Well to Erythrocytes and Are Preferentially Deposited in Glomeruli). An important step in the transfer reaction involves proteolysis of erythrocyte CR1, although the identity and mechanism of activation of the proteases involved has yet to be determined.

Reports from several laboratories using humans and non-human primates demonstrated that immune adherence of soluble IC to erythrocytes could function to rapidly and safely clear potentially pathogenic IC from the bloodstream (Cornacoff, et al., 1983 supra; Hebert, L. A. and Cosio, F. G. (1987) Kidney Int., 31, 877-885. The Erythrocyte Immune-Complex Glomerulonephritis Connection in Man; Schifferli, J. A., Ng, Y. C., Estreicher, J. and Walport, M. J. (1988) J. Immunol., 140, 899-904. The Clearance of Tetanus Toxoid Anti-Tetanus Toxoid Immune-Complexes from the Circulation of Humans—Complement and Erythrocyte Complement Receptor 1-Dependent Mechanisms; Edberg, J. C., Kujala, G. A. and Taylor, R. P. (1987) J. Immunol., 139, 1240-1244. Rapid Immune Adherence Reactivity of Nascent, Soluble Antibody DNA Immune-Complexes in the Circulation; Kimberly, R. P., Edberg J. C., Merriam, L. T., Clarkson, S. B., Unkeless, J. C., Taylor, R. P. (1989) Journal of Clinical Investigation, 84, 962. In vivo handling of soluble complement fixing Ab/dsDNA immune complexes in chimpanzees). However, for erythrocytes to function in this role, the IC must be opsonized with complement component C3b, which is recruited by antibody-coated antigen. Thus, it is clear that the efficiency of antigen clearance through this natural immune adherence mechanism is dependent on the amount of antigen, the initial antibody response, complement recruitment, and other variables associated with the overall IC system. The bifunctional conjugates described herein were designed to exploit this mechanism in a more general, non-complement dependent manner.

Taylor and coworkers have reported the use of cross-linked bispecific antibodies for clearance of human IgM from the circulation of squirrel monkeys (Reist, C. J., Liang, H. Y., Denny, D., Martin, E. N., Scheld, W. M. and Taylor, R. P. (1994) Eur. J. Immunol., 24, 2018-2025, Cross-Linked Bispecific Monoclonal-Antibody Heteropolymers Facilitate the Clearance of Human-Igm from the Circulation of Squirrel-Monkeys). This report was followed by a series of reports in 1997 in which bispecific antibodies were used to mediate the clearance of a prototype pathogen (ΦX174 phage) from the bloodstream of non-human primates (Taylor, R. P., Martin, E. N., Reinagel, M. L., Nardin, A., Craig, M., Choice, Q., Schlimgen, R., Greenbaum, S., Incardona, N. L. and Ochs, H. D. (1997) J. Immunol., 159, 4035-4044, Bispecific monoclonal antibody complexes facilitate erythrocyte binding and liver clearance of a prototype particulate pathogen in a monkey model; Taylor, R. P., Nardin, A. and Sutherland, W. M. (1997) Cancer Immunol. Immunother., 45, 152-155, Clearance of blood-borne pathogens mediated through bispecific monoclonal antibodies bound to the primate erythrocyte complement receptor; Taylor, R. P., Sutherland, W. M., Martin, E. N., Ferguson, P. J., Reinagel, M. L., Gilbert, E., Lopez, K., Incardona, N. L. and Ochs, H. D. (1987) J. Immunol., 158, 842-850, Bispecific monoclonal antibody complexes bound to primate erythrocyte complement receptor 1 facilitate virus clearance in a monkey model).

Recently, Taylor and coworkers reported obtaining protection against Pseudomonas aeruginosa in primate in vivo studies (Lindorfer, M. A., Nardin, A., Foley, P. L., Solga, M. D., Bankovich, A. J., Martin, E. N., Henderson, A. L., Price, C. W., Gyimesi, E., Wozencraft, C. P., Goldberg, J. B., Sutherland, W. M. and Taylor, R. P. (2001) J. Immunol., 167, 2240-2249, Targeting of Pseudomonas aeruginosa in the bloodstream with bispecific monoclonal antibodies). Although the bispecific antibodies in the latest work by Taylor and coworkers were reported to provide excellent protection against a bacterial challenge, these agents are not always efficient at mediating pathogen clearance.

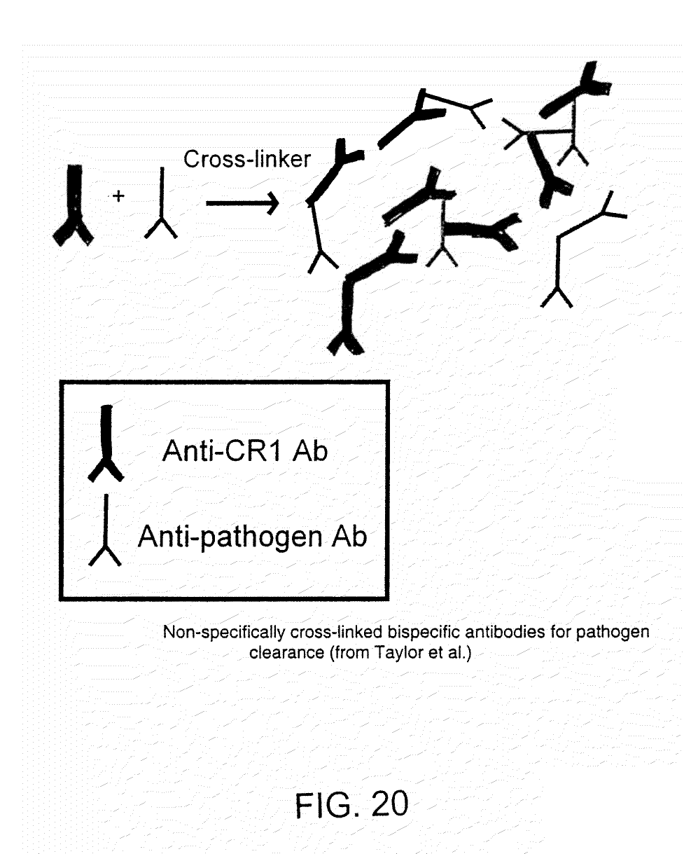

A molecular-scale analysis of these agents provides some explanatory clues for observed inefficiencies. Since these bispecific antibodies are essentially non-specifically cross-linked monoclonal antibodies, they have several disadvantageous characteristics, including a heterogeneous structure, an inability to have their structure varied in a defined manner, and the potential to cross-link through the antigen binding site, thereby preventing receptor or pathogen binding (FIG. 20). The conjugates of this invention have been designed to overcome these inherent disadvantages and provide bispecific conjugates for the efficient clearance of pathogens from the bloodstream.

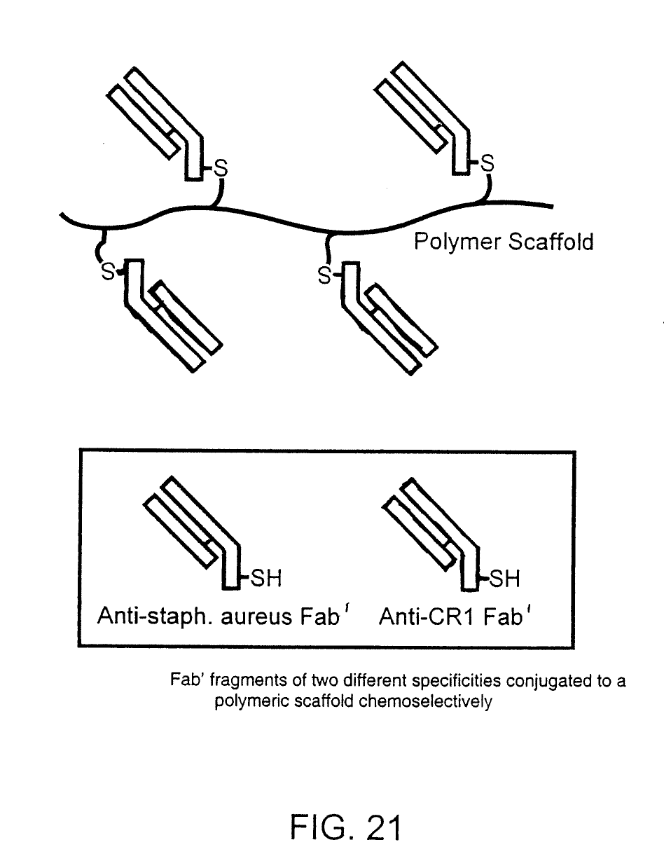

The bispecific conjugates of this example are polymeric multivalent ligands comprising Fab′ fragments of selected specificity. Specifically, the polymers are conjugates of Fab′ fragments with two different specificities, one Fab′ fragment with specificity for the CR1 receptor present on erythrocytes, and the other Fab′ fragment having specificity for a pathogen, in particular a Fab′ having specificity for Staphylococcus aureus. These Fab′ fragments have been conjugated chemoselectively to a maleimide-containing, water-soluble methyl acrylate polymer backbone (FIG. 21).

The conjugates of this invention share a significant advantage with Taylor's heteropolymers. Both approaches are quite general providing for clearance of anything (e.g., any pathogen) from the bloodstream to which a monoclonal antibody (MAb) can be raised.

However, the conjugates of this invention have a more homogeneous structure, the structure of the conjugates can be varied in a defined manner (e.g., the number, and spacing of Fab′ fragments can be varied as desired), and the structure can be selected to ensure the presence of (or maximization of) accessible antigen binding sites. An additional advantage is that the conjugates of this invention can be synthesized to have a higher binding site density as a function of valency.

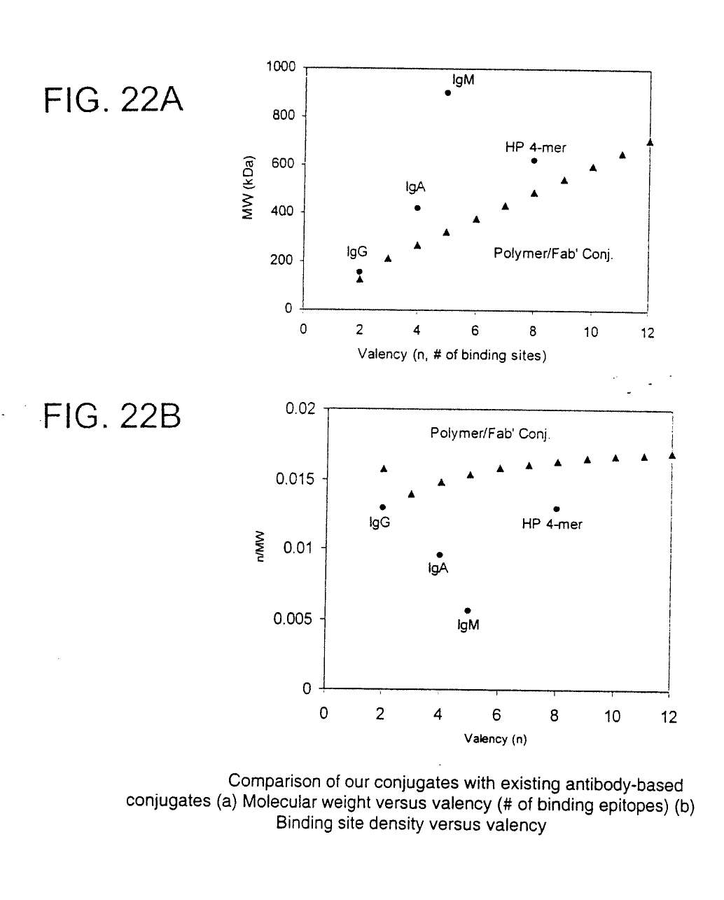

Large molecular weight conjugates can suffer from immunogenicity in vivo. A strategy that minimizes molecular weight while maintaining reasonable binding efficiency and selectivity, is therefore therapeutically advantageous. FIGS. 22A and 22B demonstrate the advantage of our conjugates in this regard. FIG. 22A demonstrates that polymer/Fab′ conjugates have a lower molecular weight as a function of valency or the number of binding sites, compared with IgG, IgA, IgM, and a hypothetical heteropolymer tetramer (HP 4-mer) prepared by the methods of Taylor and coworkers. Assigning a valency of 8 to the HP 4-mer is generous, as one or more antigen binding sites may be blocked due to the non-specific cross-linking chemistry employed in the preparation of this conjugate. Nonetheless, our conjugates are still lower in molecular weight as a function of valency.

More interestingly, FIG. 22B demonstrates that polymer/Fab′ conjugates have a larger binding site density (n/MW) as a function of valency. The polymer/Fab′ conjugate molecular weights were calculated based on data showing that a “150-mer” can easily accommodate 10-12 Fab′ fragments. Thus, this approach offers the ability to pack higher densities of biologically active epitopes into smaller molecular weight species. This ability to maximize biological activity while minimizing molecular weight is one of the guiding principles of drug design. The conjugates herein provide more active and less toxic therapeutics.

The polymeric scaffold was synthesized by first conjugating a water-soluble linker containing a Boc-protected amine to a succinimde ester-substituted polymer precursor generated by atom transfer radical polymerization (ATRP) (Scheme 12) (Godwin, A., Hartenstein, M., Muller, A. H. E. and Brocchini, S. Angew. Chem.-Int. Edit. 2001, 40, 594-597. Narrow molecular weight distribution precursors for polymer-drug conjugates.). Upon acid-mediated deprotection, the free amines were functionalized with the water-soluble maleimide-containing linker, sulfo-SMCC (Scheme 13).

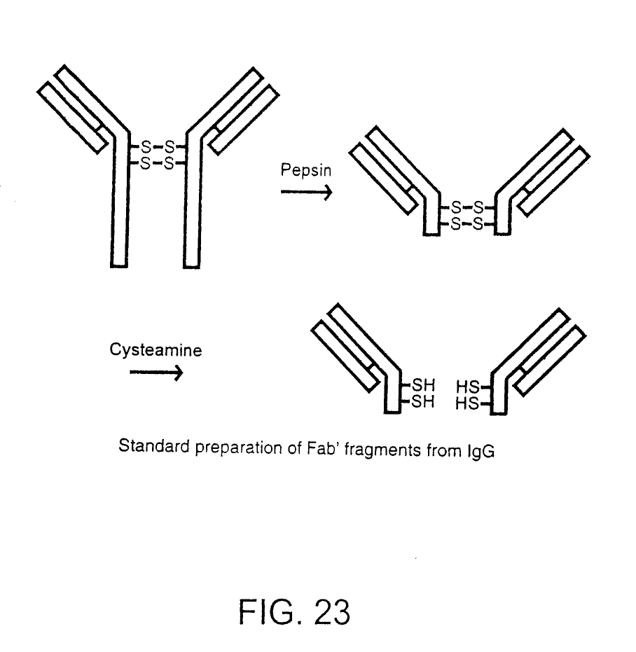

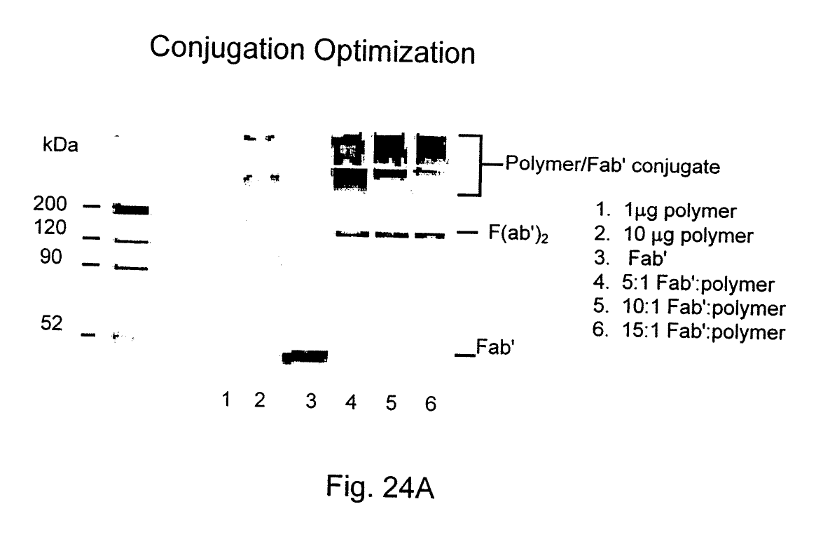

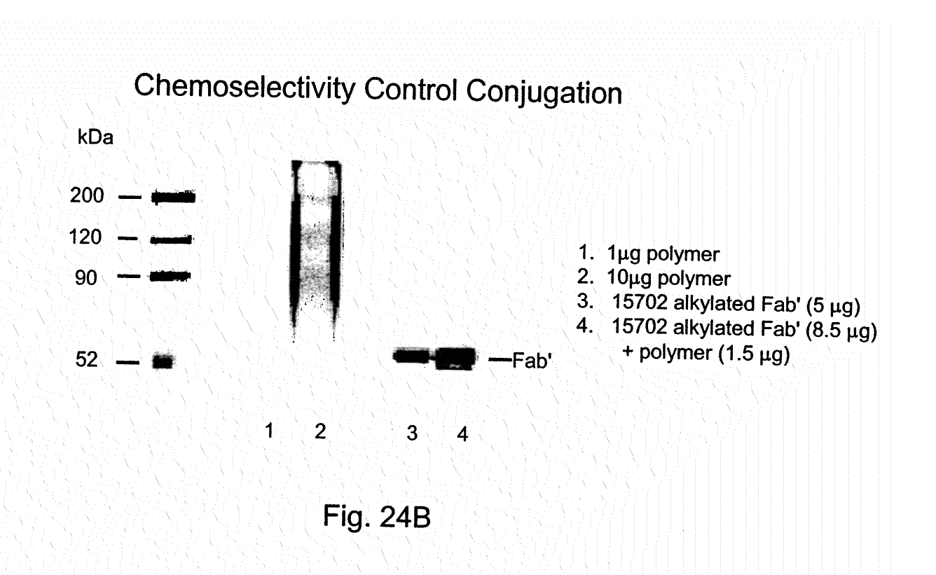

Fab′ fragments were conjugated to the polymer chemoselectively through the free thiol resulting from standard reduction of the appropriate F(ab′)₂ fragments (FIG. 23). A variety of Fab′ fragments useful in preparation of the bispecific conjugates of this example and more generally for use in preparation of multivalent ligands of this invention are available employing methods well-known in the art.

Several important characteristics of the bispecific conjugations prepared were investigated. First, the two F(ab′)₂ fragments were found to behave essentially the same with respect to susceptibility to cysteamine-mediated reduction to the corresponding Fab′ fragments and to reaction with the maleimide polymer. These results indicated that the two F(ab′)₂ fragments could be reduced, purified, and conjugated to the polymer simultaneously, enormously reducing the amount of laboratory manipulation required to prepare the desired conjugates. Secondly, in a series of conjugations in which the ratio of Fab′: polymer was increased from 5:1 to 15:1, it was demonstrated that the maleimide “150-mer” could accommodate at least 9-10 Fab′ fragments (FIG. 24A). Thirdly, a control conjugation with an alkylated Fab′ demonstrated complete chemoselectivity (FIG. 24B). Therefore, access to the binding site could be assured.

The bifunctional conjugate for pathogen clearance is based on a linear polymeric scaffold. These conjugates can be prepared in a straightforward manner to yield a polymer/Fab′ conjugates containing up to about 9-12 Fab′ fragments on a polymer having in the range of about 100-250 monomer units in length. Different Fab′ fragments were found to conjugate to the polymer with essentially the same efficiency. Finally, the conjugation was determined to be completely chemoselective, ensuring accessible antigen binding sites.

### Example 8

### Modulation of B Cell Responses Via Co-Receptors

The antigen receptors on lymphocytes play pivotal roles in controlling the balance between tolerance and immunity. In B cells, the B cell antigen receptor (BCR) transmits signals that positively or negatively regulate lymphocyte survival, growth, and differentiation. B cell activation can be modulated by co-receptors, proteins on the cell surface that positively or negatively influence the threshold for BCR activation. Synthetic multivalent ligands can be used to generate specific complexes of the BCR and co-receptors and therefore selectively enhance or attenuate B cell activation in specific clones. Investigations with antibodies have provided insight into how the clustering of proteins on the B cell surface influences the output response, but synthetic ligands offer considerable advantages. Specifically, they can interact with selected B cell populations that express specific BCRs. Moreover, synthetic ligands can be used to systematically address how alterations in the extent of receptor clustering and the organization of receptors in the cluster influence signaling.

Polymer B1 is prepared comprising one or more first ligands (CD19/CD21 ligands) that bind to the regulatory receptor CD19/CD21 complex. Polymer B2 is prepared comprising one or more second ligands (BCR ligands) that bind to the BCR. These second ligands can be in the form of specific antigens recognized by B cell clones or populations. Polymer B3 is prepared comprising one or more first and second ligands. Such B1, B2, and B3 polymers are capable of positively or negatively regulating B cell responses. Preferably such polymers are capable of positively regulating B cell responses. Without wishing to be bound by a particular theory, a possible mechanism of regulation is related to the ability of a polymer of the invention to co-cluster at least one BCR and at least one CD19/CD21 complex.

In another embodiment, multivalent ligands are prepared so as to modulate B cell responses. Without wishing to be bound by a particular theory, a possible mechanism of regulation is related to the ability of a multivalent ligand or polymer of the invention to co-cluster at least one BCR and at least one FcgammaRIIb, to co-cluster at least one BCR and at least one CD22 molecule, or to co-cluster at least one BCR, at least one FcgammaRIIb, and at least one CD22. Thus multivalent ligands or polymers are prepared as follows. Polymer B4 is prepared comprising at least one FcgammaRIIb ligand. Polymer B5 is prepared comprising at least one CD22 ligand. Polymer B6 is prepared comprising at least one BCR ligand and at least one FcgammaRIIb ligand. Polymer B7 is prepared comprising at least one BCR ligand and at least one CD22 ligand. Polymer B8 is prepared comprising at least one FcgammaRIIb ligand, at least one CD22 ligand, and at least one BCR ligand. Preferably polymers B4, B5, B6, B7, and B8 are able to inhibit BCR-mediated activation in vitro, in vivo, or both.

Ligands in this example can be recognition molecules such as Fab, Fab′, scFv, or scFv-hybrid molecules, or antigens wherein a ligand has specificity for a receptor or target molecule. Such ligands can bind and in some cases bind and stimulate a response in a host cell expressing said receptor or target molecule.

The invention also comprises methods and compositions relating to screening for modulators (inhibitors or positive regulators) of interactions and consequences of interactions using polymers and multivalent ligands of the invention.

### Example 9

### Modulation of B Cell Responses Via BCR Ligands

Antigen structure, affinity, avidity and concentration can influence BCR signaling of mature B cells. We synthesize multivalent ligands of different binding affinities, lengths and densities to test how the extent of BCR clustering influences B cell responses and to develop compositions that can regulate B cell responses.

In an embodiment, a composition comprising a multivalent ligand is prepared that is capable of negatively regulating B cell responses. Such a composition can serve as a prophylactic or therapeutic treatment for an allergic condition. In a particular embodiment an antigen of an allergen is prepared as a multivalent ligand and administered to a subject

To this end we have generated a series of multivalent ligands displaying 2,4-dinitrophenyl (DNP) groups (Scheme 15 and Table 1). Both DNP- and 2,4,6-trinitrophenyl (TNP)-substituted proteins have been shown to bind to A20/2J HLTNP cells via the BCR introduced via transfection. Although the binding constants for DNP and TNP derivatives to the target BCR are not known precisely, the TNP group is presumed to have a higher affinity on the basis of previous measurements (the binding constant for DNP is likely to be about 2-3 orders of magnitude weaker [B. G. Barisas, S. J. Singer, J. M. Sturtevant, Thermodynamics of the Binding of 2,4-Dinitrophenyl and 2,4,6-Triphenyl Haptens to the Homologous and Heterologous Rabbit Antibodies, Biochemistry 11 (1972) 2741-2744]).

To examine BCR clustering in this cell line, we synthesized multivalent ligands displaying DNP groups. The multivalent ligands that we generated vary in length and density (mole fraction) of the BCR-binding moiety (Table 1). The polymerization reactions were terminated with enol ether 212, which transfers a ketone group to the polymer terminus for subsequent attachment of a reporter group (FIG. B12) [R. M. Owen, J. E. Gestwicki, T. Young, L. L. Kiessling, Synthesis and applications of end-labeled neoglycopolymers, Org. Lett. 4 (2002) 2293-2296; E. J. Gordon, J. E. Gestwicki, L. E. Strong, L. L. Kiessling, Synthesis of end-labeled multivalent ligands for exploring cell-surface-receptor-ligand interactions, Chem. Biol. 7 (2000) 9-16; J. E. Gestwicki, C. W. Cairo, D. A. Mann, R. M. Owen, L. L. Kiessling, Selective immobilization of multivalent ligands for surface plasmon resonance and fluorescence microscopy, Analytical Biochemistry 305 (2002) 149-155].

Treatment with a hydrazide- or alkoxylamine-containing compound installs a single reporter group (e.g., biotin or a fluorophore such as fluorescein) for visualizing and/or monitoring binding [R. M. Owen, J. E. Gestwicki, T. Young, L. L. Kiessling, Synthesis and applications of end-labeled neoglycopolymers, Org. Lett. 4 (2002) 2293-2296; E. C. Rodriguez, K. A. Winans, D. S. King, C. R. Bertozzi, A Strategy for the Chemoselective Synthesis of O-Linked Glycopeptides with Native Sugar—Peptide Linkages, J. Am. Chem. Soc. 119 (1997) 9905-9906; E. C. Rodriguez, L. A. Marcaurelle, C. R. Bertozzi, Aminooxy-, Hydrazide, and Thiosemicarbazide-Functionalized Saccharides: Versatile Reagents for Glycoconjugate Synthesis, J. Org. Chem. 63 (1998) 7134-7135; V. W. Cornish, K. M. Hahn, P. G. Schultz, Site-Specific Protein Modification Using a Ketone Handle, J. Am. Chem. Soc. 118 (1996) 8150-8151; D. J. Maly, I. C. Choong, J. A. Ellman, Combinatorial target-guided ligand assembly: Identification of potent subtype-selective c-Src inhibitors, Proc. Natl. Acad. Sci. 97 (2000) 2419-2424; S. E. Cervigni, P. Dumy, M. Mutter, Synthesis of Glycopeptides and Lipopeptides by Chemoselective Ligation, Angew. Chem. Int. Ed. Engl. 35 (1996) 1230-1232].

TABLE 1
Synthetic Multivalent DNP Derivatives

| M:I^{a} | eq. DNP-Lys | % DNP^{b} | Compound |
|---|---|---|---|
| 25:1 | 0.1 | 6 | 215a |
| | 0.5 | 34 | 215b |
| | 1.0 | 59 | 215c |
| 50:1 | 0.1 | 9 | 215d |
| | 0.5 | 39 | 215e |
| | 1.0 | 73 | 215f |
| 100:1 | 0.1 | 6 | 215g |
| | 0.5 | 37 | 215h |
| | 1.0 | 63 | 215i |
| 250:1 | 0.1 | 8 | 215j |
| | 0.5 | 34 | 215k |
| | 1.0 | 64 | 215m |

^{a}M:I refers to the monomer to initiator ratio used in the polymerization reaction. The average lengths of polymers generated using this procedure are typically slightly longer than the monomer to initiator ratio predicts.
^{b}Percent DNP values were determined using 1H NMR integration. The polymer numbering scheme is from Scheme 15.

Those of ordinary skill in the art will appreciate in view of the descriptions herein that there are a variety of alternative structures, methods, procedure and techniques that can be readily applied or adapted to the practice of this invention other than those that have been specifically exemplified. It will be appreciated that there are a wide variety of designs for and methods for preparation of multivalent ligands with properties as described herein. It will also be appreciated that there are a wide variety of molecular scaffolds available for the productive presentation of BRE and/or SRE as well as a wide variety of BRE and/or SRE that can be applied or adapted to the methods described herein.

All of the references cited herein are incorporated by reference herein in their entirety and to the extent that they are not inconsistent with the disclosures herein. The cited references are incorporated by reference herein in particular for any description regarding the synthesis of multivalent ligands and particularly synthesis by ROMP or ATRP methods and for description regarding the selection of signal groups and preparation of signal groups, particularly chemoattractants, epitopes, antibodies, antibody fragments, N-formyl peptides for a given application and or for the selection of binding groups and the preparation of binding groups, particularly metal-chelating groups, antibodies and antibody fragments for a given application. References are also incorporated by reference herein to provide additional details of assays, including functional assays, to examine the function of BRE, SRE and mutivalent ligands containing these groups. References are also incorporated by reference herein for the selection of and preparation of FE groups that are useful in the multivalent ligands and applications thereof.

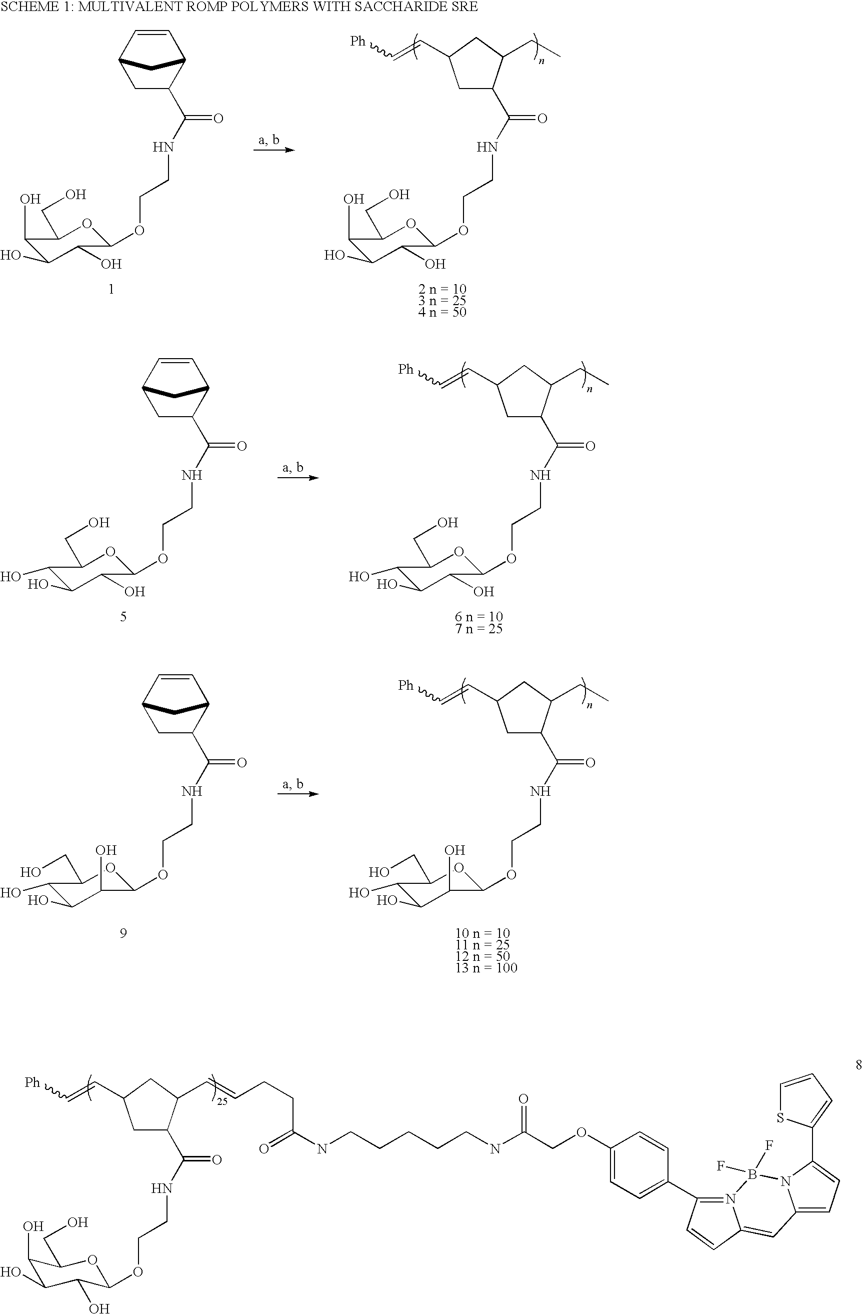

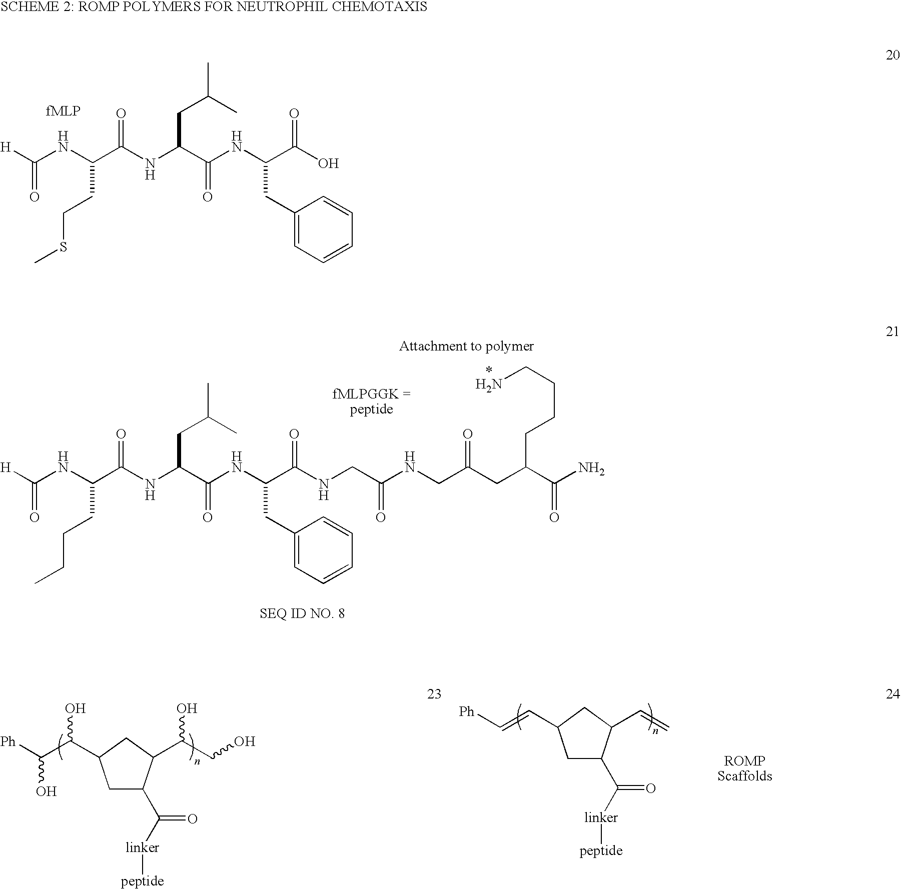

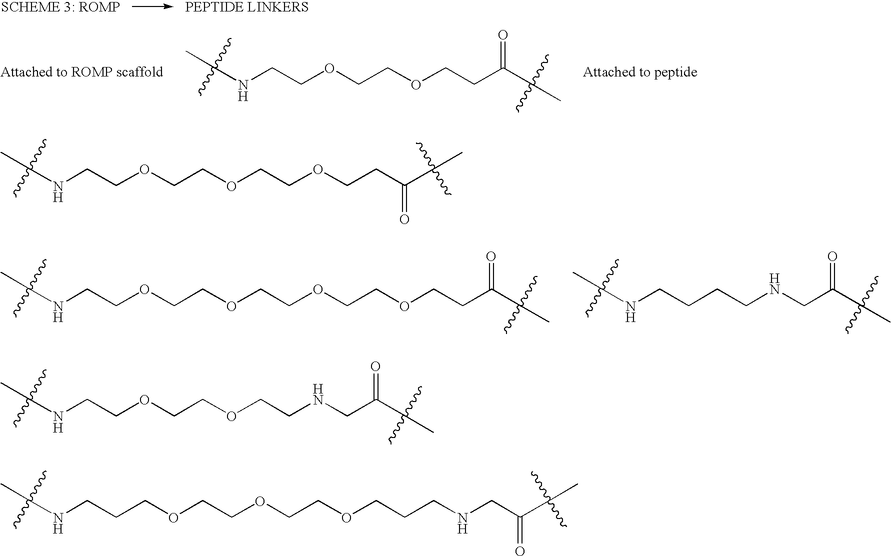

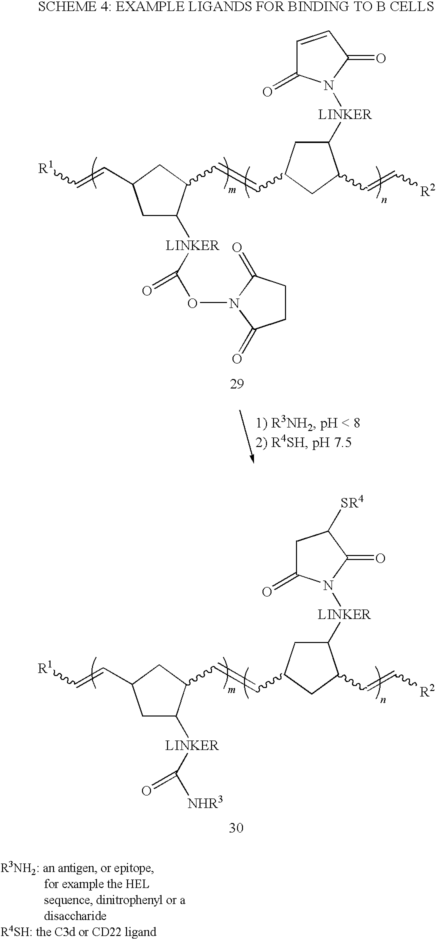

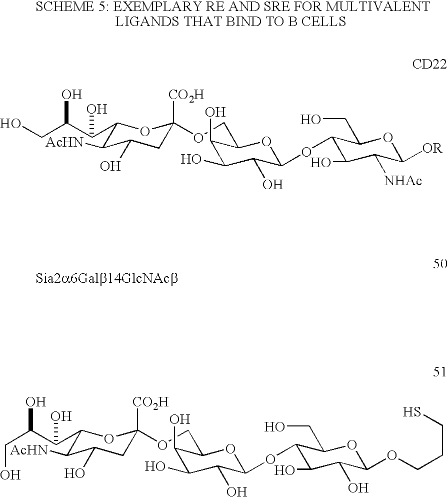

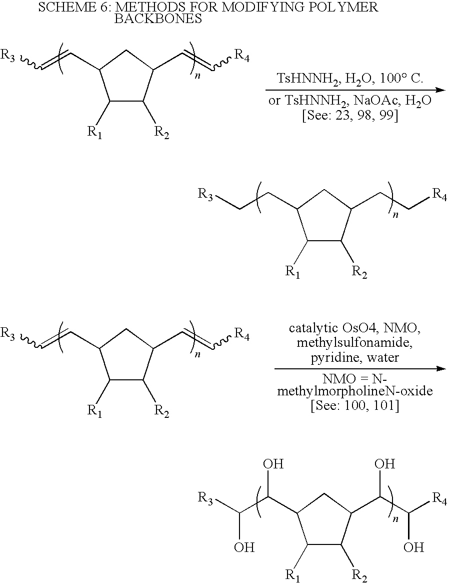

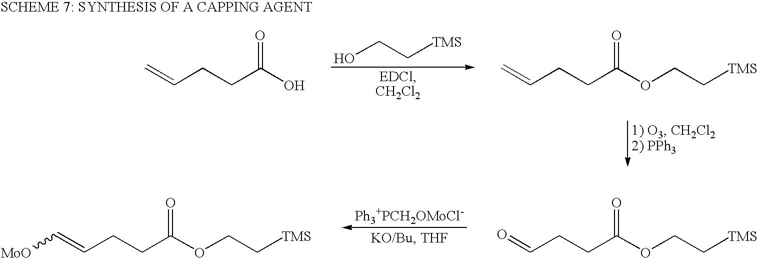

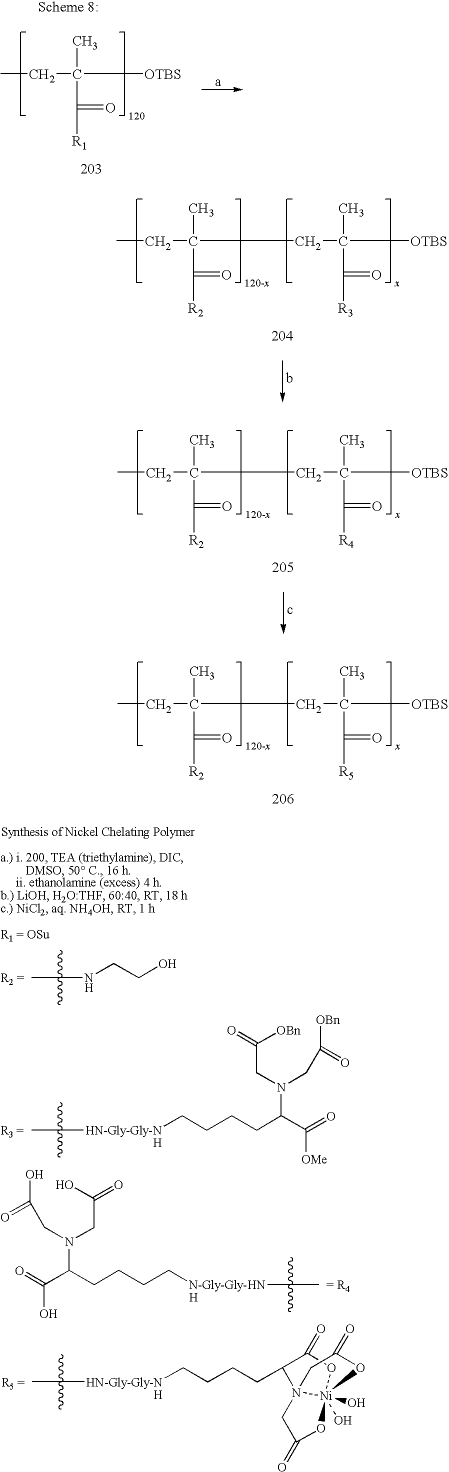

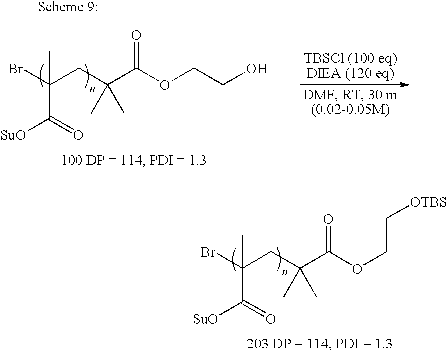

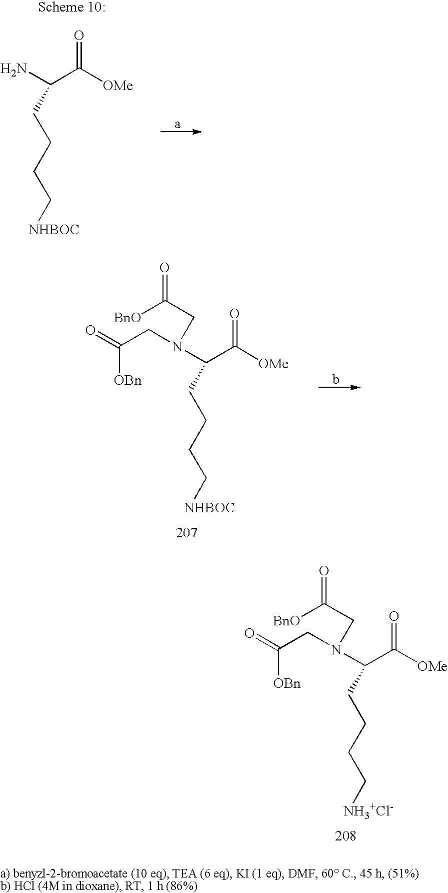

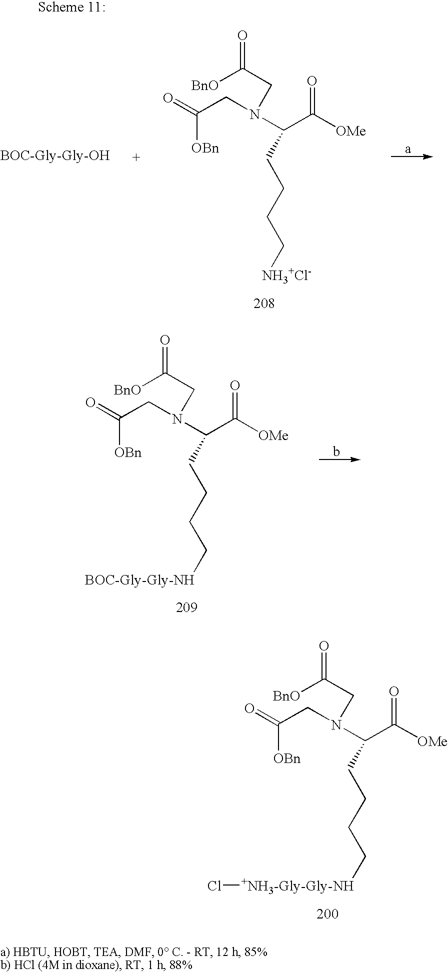

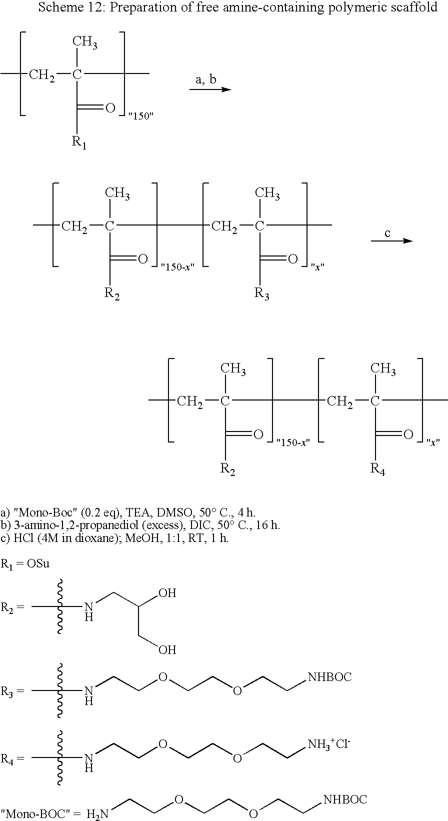

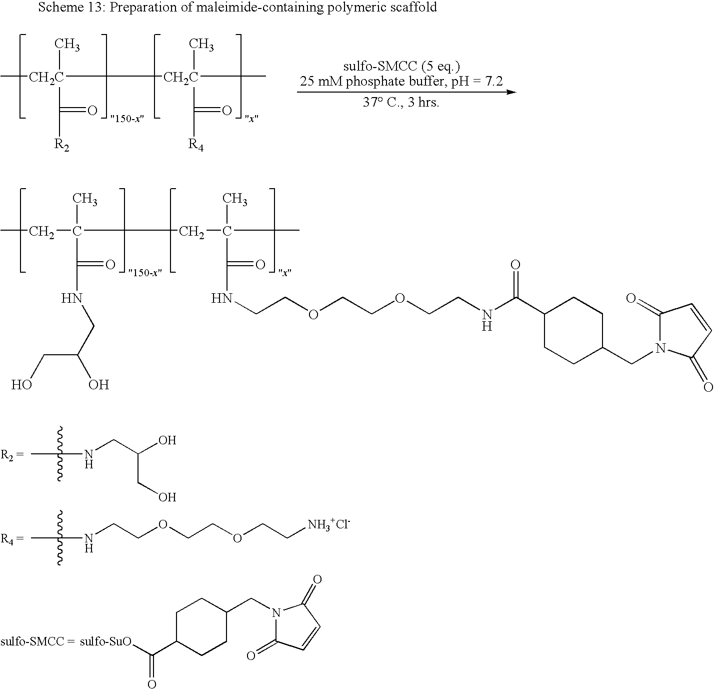

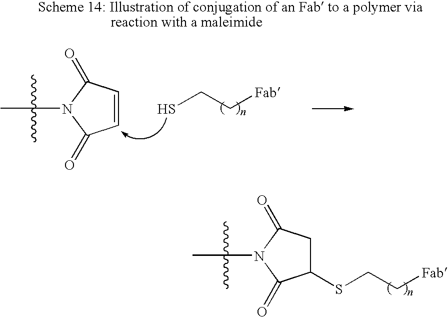

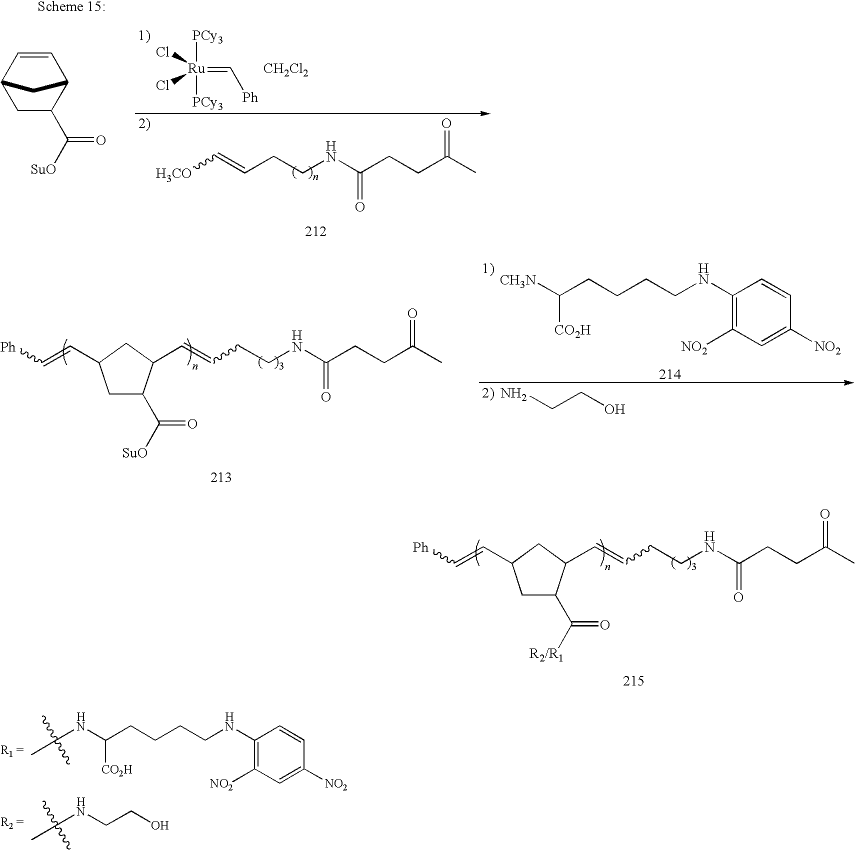

1. A method for inducing a biological response in a biological system comprising one or more receptors which comprises the step of introducing into the biological system a multivalent ligand which comprises a plurality of signal recognition elements recognized by at least one of the receptors and bonded to a molecular scaffold.

2-12. (canceled)

13. The method of claim 1 wherein the biological system is a eukaryotic cell of the immune system.

14. The method of claim 13 wherein the eukaryotic cell is a lymphocyte or a leukocyte.

15-20. (canceled)

21. The method of wherein the biological response is an intracellular signal in a biological system which is a cell.

22. (canceled)

23. The method of claim 13 wherein the cell is a B-cell or a T-cell.

24. The method of claim 23 wherein the multivalent ligand comprises a signal recognition element that is an epitope foreign to the organism from which the B-cell or T-cell originates.

25. The method of claim 24 wherein the multivalent ligand further comprises a signal recognition element that binds to a cell surface receptor of a B-cell or a T-cell.

26. The method of claim 25 wherein the multivalent ligand functions to enhance immunogenicity of the foreign epitope.

27. The method of claim 25 wherein the multivalent ligand comprises a signal recognition element that is an epitope recognized as a self epitope by the B-cell or T-cell.

28. The method of claim 27 wherein the multivalent ligand further comprises one or more different signal recognition element that bind to one or more cell surface receptors of a B-cell or a T-cell.

29. The method of claim 27 wherein the multivalent ligand functions to sensitize the cell to the self epitope.

30. The method of claim 29 wherein the multivalent ligand further comprises one or more different signal recognition elements that bind to one or more cell surface receptors of a B-cell or a T-cell.

31-32. (canceled)

33. The method of claim 30 wherein the multivalent ligand further comprises one or more signal recognition elements that bind to one or more different cell surface receptors of a B-cell or a T-cell.

34. The method of claim 32 wherein the multivalent ligand functions to tolerize the cell to the self epitope that is recognized as a foreign epitope by the B-Cell or T-cell.

35-55. (canceled)

56. The method of claim 1 wherein the multivalent ligand comprises about 25 or more signal recognition elements.

57. The method of claim 1 wherein the multivalent ligand comprises about 100 or more signal recognition elements.

58-94. (canceled)

95. A method for inducing or enhancing induction of a cellular response which comprises the steps of:
forming a multivalent ligand which comprises a plurality of signal recognition elements which individually bind to the cell and induce the cellular response and contacting the cells with the multivalent ligand in an amount sufficient to enhance the cellular response.

96-127. (canceled)

128. A method for inducing a biological response in a biological system comprising one or more receptors which comprises the step of introducing into the biological system a multivalent ligand which comprises a plurality of signal recognition elements bonded to a molecular scaffold wherein the signal recognition elements are recognized by at least one of the receptors
wherein the biological response is immune adherence and the biological system comprises erythrocytes and wherein the polymer is an ATRP polymer or a ROMP polymer.

129. The method of claim 128 wherein the receptor is a receptor on the erythrocytes.

130. The method of claim 129 wherein the receptor is CR1 and the signal recognition element is a Fab′ fragment.


SEQUENCE LISTING (8 sequences)

SEQ ID NO: 1 - protein, 15 aa, Artificial Sequence
  Synthetic construct  peptide
NGMNAWVAWRNRCKG

SEQ ID NO: 2 - protein, 16 aa, Artificial Sequence
  Synthetic construct  peptide
KNRWEDPGKQLYNVEA

SEQ ID NO: 3 - protein, 15 aa, Artificial Sequence
  Synthetic construct  peptide
GDGNGMNAWVAWRNR

SEQ ID NO: 4 - protein, 16 aa, Artificial Sequence
  Synthetic construct peptide
DGNGMNAWVAWRNRGK

SEQ ID NO: 5 - protein, 17 aa, Artificial Sequence
  Synthetic construct  peptide
CKNRWEDPGKQLYNVEA

SEQ ID NO: 6 - protein, 17 aa, Artificial Sequence
  Synthetic construct  peptide
KNRWEDPGKQLYNVEAC

SEQ ID NO: 7 - protein, 25 aa, Artificial Sequence
  Synthetic construct  peptide
ELAAAMKRHGLDNYRGYSLGNWVCA

SEQ ID NO: 8 - protein, 6 aa, Artificial Sequence
  Synthetic construct  peptide; Methionine residue is N-formylated.
MLFGGK